(19) United States
(12) Patent Application Publication
(10) Pub. No.: US 2026/0306523 A1
(43) Pub. Date: Oct. 8, 2026

(54) COMPOUNDS AND COMPOSITIONS FOR TREATING GLUCOSE HYPOMETABOLIC DISORDERS

(71) Applicant: Senovia Biosciences LLC, Florence, AL (US)

(72) Inventor: Joel R. Anderson, II, Florence, AL (US)

(21) Appl. No.: 19/630,033

(22) Filed: Mar. 26, 2026

Related U.S. Application Data
(60) Provisional application No. 63/778,042, filed on Mar. 26, 2025.
(60) Provisional application No. 63/920,843, filed on Nov. 19, 2025.

(51) Int. Cl. A61K 31/7024; A61K 31/185; A61K 31/22; A61K 31/223; A61K 31/417; A61P 25/28; C07C 67/08; C07C 69/716; C07C 231/02; C07C 235/74; C07C 309/15; C07D 233/64; C07H 13/04

(52) U.S. Cl. CPC A61K 31/7024; A61K 31/185; A61K 31/22; A61K 31/223; A61K 31/417; A61P 25/28; C07C 67/08; C07C 69/716; C07C 231/02; C07C 235/74; C07C 309/15; C07D 233/64; C07H 13/04

(57) ABSTRACT

The present disclosure provides systems, methods, compositions, and algorithms for treating a subject diagnosed with a glucose hypometabolic disorder (e.g., Alzheimer's disease, Parkinson's disease, or heart failure).

### CROSS-REFERENCE TO RELATED APPLICATIONS

This application claims priority to and the benefit of U.S. Provisional Patent Application No. 63/778,042, filed Mar. 26, 2025, and U.S. Provisional Patent Application No. 63/920,843, filed Nov. 19, 2025, the contents of each of which are hereby incorporated by reference in their entirety.

### BRIEF SUMMARY

Described herein are compounds and compositions for treating a common underlying metabolic mechanism in diverse diseases. Specifically, the compounds and compositions described herein are useful for treating glucose hypometabolism and the resultant cellular energy crisis found in neurodegenerative, cardiovascular, and metabolic disorders that were previously considered unrelated.

### FIELD OF THE INVENTION

This application relates to compounds and compositions, specifically ketone bodies conjugated to biologically active carriers, for treating neurological, cardiovascular, metabolic, psychiatric, inflammatory, and neuromuscular disorders characterized by glucose hypometabolism.

### BACKGROUND

Diseases and disorders have historically been classified based on presenting symptoms and affected organs rather than underlying molecular and metabolic mechanisms. For example, the symptomatic presentations of Alzheimer's disease (memory loss), heart failure (reduced cardiac output), and Parkinson's disease (motor dysfunction) appear entirely distinct, masking their shared metabolic pathophysiology. A unifying feature of these conditions is impaired glucose utilization at the cellular level, often due to reduced glucose uptake or utilization in affected regions. Despite decades of research in each of these disease areas, this unifying mechanism—glucose hypometabolism—has remained unrecognized due to several key factors, including entrenched medical specialization silos, lack of previous cross-disease metabolic comparisons, and technological limitations to precisely measure and quantify tissue-specific glucose metabolism. In Alzheimer's disease, the dominant amyloid hypothesis has guided therapeutic development for decades, despite consistent failures. In Parkinson's disease, focus has remained on dopaminergic therapies despite their inability to modify disease progression. In heart failure, hemodynamic approaches have predominated despite incomplete efficacy. There remains a need to develop a unifying therapeutic approach to address the common underlying pathophysiology—glucose hypometabolism and resultant cellular energy crisis—behind these and other traditionally considered distinct and unrelated disorders, including neurodegenerative, cardiovascular, and metabolic disorders.

Ketone bodies, namely β-hydroxybutyrate (βHB) and acetoacetate (AcAc), represent an alternative energy source for the brain that can bypass defects in glucose metabolism. During fasting or ketogenic diets, the liver produces ketone bodies which can supply up to two-thirds of the brain's energy. Importantly, ketones produce more ATP per unit oxygen than glucose and generate fewer reactive oxygen species (ROS) during metabolism, thereby offering a dual benefit of efficient energy and reduced oxidative stress. Preclinical and clinical evidence suggests ketone supplementation can lead to significant cognitive or metabolic improvements in patients with mild cognitive impairment and Alzheimer's disease, improvement in non-motor symptoms in patients with Parkinson's disease, and reduction in hospitalization risk in patients with heart failure.

However, administration of ketone bodies in therapeutic amounts presents formulation and stability challenges. Free ketone bodies are rapidly cleared from circulation, limiting their therapeutic efficacy. Additionally, high concentrations of free ketone salts can cause gastrointestinal distress and electrolyte imbalances. Prior approaches to ketone supplementation have included ketone salts, ketone esters, medium-chain triglycerides, and specialized ketogenic diets. However, these approaches suffer from limitations including poor palatability, gastrointestinal side effects, limited bioavailability, and challenges in achieving sustained therapeutic ketosis. Therefore, there remains a need for novel compositions that can effectively deliver therapeutic quantities of ketone bodies to tissues affected by glucose hypometabolism, with improved bioavailability, targeted delivery, and minimal side effects.

### SUMMARY OF THE DISCLOSURE

Disclosed herein, in certain embodiments, are compounds and compositions for treating a subject diagnosed with a glucose hypometabolic disorder (e.g., Alzheimer's disease, Parkinson's disease, heart failure, or other diseases and disorders described herein).

In one aspect provided is a compound of Formula (A):

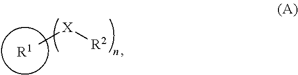

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In certain embodiments, R¹ is selected from the group consisting of trehalose, myo-inositol, D-ribose, inulin, creatine, glycine, carnitine, taurine, glycerol, allulose, aspartic acid, and carnosine.

In another aspect, the invention provides pharmaceutical formulations comprising a compound of Formula (A) and pharmaceutically acceptable excipients suitable for oral, enteral, parenteral, or intravenous delivery.

In yet another aspect, the invention provides methods for treating glucose hypometabolic disorders by administering to a patient an effective amount of a compound of Formula (A), wherein the disorders include Alzheimer's disease, Parkinson's disease, heart failure, type 2 diabetes, epilepsy, traumatic brain injury, amyotrophic lateral sclerosis, cancer cachexia, metabolic syndrome, and any condition exhibiting impaired glucose utilization.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a bar graph showing dose-dependent effects of acetoacetate (AcAc) on neuronal viability in Alzheimer's disease (AD) neurons. Neurons were treated with amyloid beta (Aβ1-42) and increasing concentrations of AcAc (approximately 0.1 mM, 0.5 mM, 1 mM, 10 mM, and 100 mM). Cell viability was measured using a luminescence-based live/dead assay (CellTiter-Glo). Increasing concentrations of AcAc progressively improve viability relative to untreated AD neurons exposed to Aβ, approaching levels observed in healthy control neurons.

FIG. 2 is a bar graph showing dose-dependent effects of acetoacetate (AcAc) on reactive oxygen species (ROS) levels in AD neurons. Neurons were treated with amyloid beta (Aβ1-42) and increasing concentrations of AcAc (approximately 0.1 mM to 100 mM). ROS levels were measured using MitoSOX staining. Increasing concentrations of AcAc reduce MitoSOX fluorescence intensity relative to untreated AD neurons, indicating a dose-dependent reduction in oxidative stress.

FIG. 3 is a bar graph showing effects of multiple ketone bodies, including D-β-hydroxybutyrate (D-BHB), L-β-hydroxybutyrate (L-BHB), and acetoacetate (AcAc), on ROS levels in AD neurons under amyloid beta stimulation. Ketone treatment reduces ROS relative to untreated AD neurons, demonstrating a class-wide effect of ketones on oxidative stress reduction.

FIG. 4 is a set of fluorescence microscopy images showing mitochondrial structure and oxidative stress in neurons under amyloid beta-induced conditions. Neurons were treated with 1 μM amyloid beta (Aβ1-42) in the presence or absence of β-hydroxybutyrate (β-OHB) at concentrations of approximately 250 μM and 500 μM. MitoTracker staining (green) indicates mitochondrial structure and integrity, while MitoSOX staining (red) indicates mitochondrial reactive oxygen species (ROS) production. Ketone-treated cells exhibit reduced MitoSOX signal intensity and improved mitochondrial morphology relative to amyloid-treated cells without ketone treatment, indicating reduced oxidative stress and preservation of mitochondrial integrity.

FIG. 5 is a bar graph quantifying mitochondrial oxidative stress in neurons under amyloid beta-induced conditions. Neurons were treated with 1 μM amyloid beta (Aβ1-42) in the presence or absence of β-hydroxybutyrate (β-OHB) at concentrations of approximately 250 μM and 500 μM. Reactive oxygen species (ROS) levels were measured using MitoSOX staining and normalized to MitoTracker signal. Treatment with β-OHB reduced MitoSOX fluorescence intensity relative to amyloid-treated cells without ketone treatment, indicating reduced mitochondrial oxidative stress. Lower values correspond to reduced oxidative stress.

FIG. 6 is a bar graph showing dose-dependent effects of acetoacetate (AcAc) on intracellular ATP levels in Alzheimer's disease (AD) neurons. Neurons were treated with 1 μM amyloid beta (Aβ1-42) and increasing concentrations of AcAc (approximately 0.1 mM, 0.5 mM, 1 mM, 10 mM, and 100 mM). Intracellular ATP levels were measured using an ATP-dependent luminescence assay and are presented relative to healthy control neurons. AD neurons exhibit reduced ATP levels relative to healthy controls, while treatment with increasing concentrations of AcAc results in a dose-dependent increase in ATP levels, indicating restoration of cellular energy metabolism.

FIG. 7 is a bar graph showing neuronal viability under basal and amyloid beta-stimulated conditions in AD neurons. Neurons were treated with 1 μM amyloid beta (Aβ1-42) in the presence or absence of ketone bodies, including D-β-hydroxybutyrate (D-BHB), L-β-hydroxybutyrate (L-BHB), and acetoacetate (AcAc), at approximately 5 mM for 14 days. Cell viability was measured using a CellTiter-Glo Live/Dead assay. Amyloid beta exposure reduces viability relative to basal conditions, while ketone treatment restores viability to varying extents.

FIG. 8 is a bar graph showing apoptosis levels in AD neurons as measured by Annexin V staining. Neurons were treated with 1 μM amyloid beta (Aβ1-42) in the presence or absence of ketone bodies (D-BHB, L-BHB, and AcAc) at approximately 5 mM for 14 days. The percentage of Annexin V-positive cells is increased following amyloid beta exposure and reduced following ketone treatment, indicating decreased apoptotic signaling.

FIG. 9 is a graph showing oxygen consumption rate (OCR) over time in AD neurons as measured by a Seahorse XF Mito Stress Test. Neurons were treated with ketone bodies (D-BHB, L-BHB, and AcAc). OCR was measured across sequential additions of mitochondrial modulators, including oligomycin, FCCP, and rotenone/antimycin A. Ketone-treated neurons exhibit increased mitochondrial respiration relative to untreated AD neurons.

FIG. 10 is a bar graph showing ATP-linked respiration in AD neurons. Neurons were treated with ketone bodies (D-BHB, L-BHB, and AcAc) and ATP concentration was measured. Ketone-treated neurons exhibit increased ATP relative to untreated AD neurons.

FIG. 11 is a bar graph showing basal respiratory capacity in AD neurons. Neurons were treated with ketone bodies (D-BHB, L-BHB, and AcAc). Basal respiration was determined using Seahorse XF Mito Stress Test analysis. Ketone-treated neurons exhibit increased basal respiratory capacity relative to untreated AD neurons, indicating improved mitochondrial function.

FIG. 12 is a bar graph showing neuronal viability in Parkinson's disease (PD) neurons. Neurons were treated with ketone bodies (D-BHB, L-BHB, and AcAc). Cell viability was measured using a CellTiter-Glo Live/Dead assay. PD neurons exhibit reduced viability relative to healthy controls, and ketone treatment improves viability, with AcAc demonstrating a comparatively strong effect.

FIG. 13 is a bar graph showing apoptosis levels in PD neurons as measured by Annexin V staining. Neurons were treated with ketone bodies (D-BHB, L-BHB, and AcAc). PD neurons exhibit increased percentages of Annexin V-positive cells relative to healthy controls, and ketone treatment reduces apoptotic signaling.

FIG. 14 is a bar graph showing reactive oxygen species (ROS) levels in PD neurons as measured by MitoSOX staining. Neurons were treated with ketone bodies (D-BHB, L-BHB, and AcAc). PD neurons exhibit elevated ROS levels relative to healthy controls, and ketone treatment reduces ROS levels to varying extents.

FIG. 15 is a graph showing oxygen consumption rate (OCR) over time in PD neurons as measured by a Seahorse XF Mito Stress Test. Neurons were treated with ketone bodies (D-BHB, L-BHB, and AcAc). OCR was measured across sequential additions of mitochondrial modulators, including oligomycin, FCCP, and rotenone/antimycin A. Ketone-treated neurons exhibit increased mitochondrial respiration relative to untreated PD neurons.

FIG. 16 is a bar graph showing ATP production in PD neurons. Neurons were treated with ketone bodies (D-BHB, L-BHB, and AcAc). ATP-linked respiration was derived from Seahorse XF Mito Stress Test measurements. Ketone treatment increases ATP production relative to untreated PD neurons.

FIG. 17 is a bar graph showing basal respiratory capacity in PD neurons. Neurons were treated with ketone bodies (D-BHB, L-BHB, and AcAc). Basal respiration was determined using Seahorse XF Mito Stress Test analysis. Ketone treatment increases basal respiratory capacity relative to untreated PD neurons.

FIG. 18 is a bar graph showing uptake of amyloid beta (Aβ1-42) by microglia treated with ketone esters. Microglia were treated with ketone esters and exposed to fluorescently labeled Aβ1-42 for approximately 1 hour. Following acid wash to remove non-internalized amyloid, intracellular fluorescence was measured. Increased fluorescence intensity indicates enhanced internalization of amyloid beta in ketone-treated microglia relative to untreated controls.

FIG. 19 is a bar graph showing clearance of amyloid beta (Aβ1-42) by microglia following ketone treatment. Microglia were treated with ketone esters and exposed to fluorescently labeled Aβ1-42 for approximately 24 hours. Residual extracellular amyloid beta was quantified by fluorescence analysis. Reduced fluorescence intensity indicates decreased residual amyloid beta and enhanced clearance relative to untreated controls.

FIG. 20 is a line graph showing the relative kinetics of amyloid beta (Aβ1-42) clearance in microglia over time. Microglia were treated with acetoacetate (AcAc) and exposed to amyloid beta, and clearance was monitored over a time course of approximately 0 to 30 hours. Ketone-treated microglia exhibit enhanced uptake at early time points and accelerated clearance relative to vehicle-treated controls, resulting in improved overall amyloid clearance kinetics.

FIG. 21 displays a graph demonstrating the mean time-plasma BHB concentration following a representative compound of Formula (A), TrehaAce, PO dosing in mice.

### DETAILED DESCRIPTION

Provided herein, in certain embodiments, are systems, methods, compositions, and algorithms for treating a subject diagnosed with a glucose hypometabolic disorder (e.g., Alzheimer's disease, Parkinson's disease, heart failure, or other diseases and disorders described herein). The contemplated systems, methods, compositions, and algorithms disclosed herein are useful in a diagnostic-therapeutic platform for identifying and treating glucose hypometabolism and resultant cellular energy crisis in subjects by quantitatively measuring tissue-specific glucose metabolism deficits and administering precisely calibrated exogenous ketone formulations to compensate for these deficits.

### Certain Terminology

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

The articles “a” and “an” are used herein to refer to one or to more than one (i.e. at least one) of the grammatical objects of the article.

### Chemical Definitions

Definitions of specific functional groups and chemical terms are described in more detail below.

In some embodiments, compounds described herein comprise one or more asymmetric centers, and thus exist in various isomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. The disclosure additionally encompasses compounds described herein as individual isomers substantially free of other isomers, and alternatively, as mixtures of various isomers.

In some embodiments, compounds described herein also comprise one or more isotopic substitutions. For example, in some embodiments, H is in any isotopic form, including ¹H, ²H (D or deuterium), and ³H (T or tritium); C is any isotopic form, including ¹²C, ¹³C, and ¹⁴C; O is in any isotopic form, including ¹⁶O and ¹⁸O; F is in any isotopic form, including ¹⁸F and ¹⁹F; and the like.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁-C₂₀ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). Examples of C₁-C₆ alkyl groups include methyl, ethyl, propyl, isopropyl, butyl, isobutyl, pentyl, hexyl, and the like.

As used herein, “alkylene,” refers to a divalent radical of an alkyl group. When a range or number of carbons is provided for a particular “alkylene” group, it is understood that the range or number refers to the range or number of carbons in the linear carbon divalent chain. In some embodiments, “alkylene” group is substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In some embodiments, in heteroaryl groups that contain one or more nitrogen atoms, the point of attachment is a carbon or nitrogen atom, as valency permits.

In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl.

Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively.

The term “cycloalkyl” refers to a monovalent saturated cyclic, bicyclic, or bridged cyclic (e.g., adamantyl) hydrocarbon group of 3-12, 3-10, 3-8, 4-8, or 4-6 carbons, referred to herein, e.g., as “C₃-C₁₀cycloalkyl,” derived from a cycloalkane. Exemplary cycloalkyl groups include, but are not limited to, cyclohexanes, cyclopentanes, cyclobutanes and cyclopropanes.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, and sulfur (“3-10 membered heterocyclyl”). In some embodiments, in heterocyclyl groups that contain one or more nitrogen atoms, the point of attachment is a carbon or nitrogen atom, as valency permits. In some embodiments, a heterocyclyl group is either be monocyclic (“monocyclic heterocyclyl”) or a fused, bridged or spiro ring system such as a bicyclic system (“bicyclic heterocyclyl”), and is saturated or is partially unsaturated. In some embodiments, heterocyclyl bicyclic ring systems include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more phenyl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” are used interchangeably.

In some embodiments, a heterocyclyl group is a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In some embodiments, a heterocyclyl group is a 3-7 membered non-aromatic ring system having ring carbon atoms and 1-4 ring heteroatoms, wherein each heteroatom is independently selected from nitrogen, oxygen, and sulfur (“3-7 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

“Hetero” when used to describe a compound or a group present on a compound means that one or more carbon atoms in the compound or group have been replaced by a nitrogen, oxygen, or sulfur heteroatom. In some embodiments, hetero is applied to any of the hydrocarbyl groups described above such as alkyl, e.g., heteroalkyl; cycloalkyl, e.g., heterocyclyl; aryl, e.g., heteroaryl; and the like having from 1 to 5, and particularly from 1 to 3 heteroatoms.

The terms “halo” and “halogen” as used herein refer to an atom selected from fluorine (fluoro, —F), chlorine (chloro, —Cl), bromine (bromo, —Br), and iodine (iodo, —I). In certain embodiments, the halo group is either fluoro or chloro.

The term “haloalkyl” includes mono, poly, and perhaloalkyl groups substituted with one or more halogen atoms where the halogens are independently selected from fluorine, chlorine, bromine, and iodine.

The term “oxo” as used herein refers to ═O.

The term “hydroxyl” as used herein refers to —OH.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms are substituted or unsubstituted as valency permits, and include primary, secondary, tertiary, and quarternary nitrogen atoms.

These and other exemplary substituents are described in more detail in the Detailed Description, Examples, and Claims. The disclosure is not intended to be limited in any manner by the above exemplary listing of substituents.

### Other Definitions

As used herein, the term “about” when modifying a numeric value means within a reasonable range of the stated value, typically ±10% unless otherwise specified. This term accounts for normal experimental or biological variability around the precise value.

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” “individual” and “subject” are used interchangeably herein. None of the terms require the supervision of medical personnel.

The terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition.

As used herein, and unless otherwise specified, a “therapeutically effective amount” or an “effective amount” of a compound is an amount sufficient to provide a therapeutic benefit in the treatment of a disease, disorder or condition, or to delay or minimize one or more symptoms associated with the disease, disorder or condition. A therapeutically effective amount or an effective amount of a compound means an amount of therapeutic agent, alone or in combination with other therapies, which provides a therapeutic benefit in the treatment of the disease, disorder or condition. In some embodiments, the term “therapeutically effective amount” or “effective amount” encompasses an amount that improves overall therapy, reduces or avoids symptoms or causes of disease or condition, or enhances the therapeutic efficacy of another therapeutic agent.

As used herein, the term “ketone body”, “exogenous ketone” or “exogenous ketone body” means a compound or species which is a ketone or a ketone body precursor, that is, a compound or species which is a precursor to a ketone and which may be converted or metabolized to a ketone. Ketone bodies are produced when fatty acids levels are raised in the body and are metabolized by the body for energy. Ketone bodies have been disclosed as being suitable for reducing the levels of free fatty acids circulating in the plasma of a subject and that ingestion of ketone bodies can lead to various clinical benefits, including an enhancement of cognitive performance and treatment of cardiovascular conditions, diabetes and treatment of mitochondrial dysfunction disorders and in treating muscle fatigue and impairment. Examples of ketone bodies include, but are not limited to, beta-hydroxybutyrate (“βHB”) and acetoacetate (“AcAc”).

For consistency, the terms “administering” and “delivering” (with respect to providing a therapeutic composition to a subject) are used interchangeably herein. Likewise, terms like “exogenous ketone therapy,” “ketone composition,” or “ketone formulation” refer to the use of one or more ketone compounds (or precursors) administered to the subject as described.

As used herein, the term “glucose hypometabolic disorder” refers to any disease, disorder, or condition in which one or more tissues exhibit a chronically reduced capacity to utilize glucose relative to normal physiology. Examples of glucose hypometabolic disorders include, but are not limited to, Alzheimer's disease, Parkinson's disease, heart failure, type 2 diabetes, epilepsy, traumatic brain injury, amyotrophic lateral sclerosis, cancer cachexia, metabolic syndrome, and any other condition in which the subject's tissue exhibits a quantifiable reduction in glucose utilization.

As used herein, the term “glucose metabolism deficit,” abbreviated as “G_Diff,” refers to the quantitative shortfall in glucose utilization by a tissue (or entire subject) compared to a normal or baseline value. G_Diff may be expressed as a percentage below a normal glucose metabolic rate, an absolute deficit in terms of glucose consumption (e.g., milligrams per deciliter lower than normal), or an energy equivalent. For example, a G_Diff of 20% indicates the tissue is only achieving about 80% of the glucose uptake/usage observed in a healthy reference under the same conditions.

The term “module” (as in “diagnostic module,” “computational module,” etc.) refers broadly to a component or combination of components—implemented in hardware, software, firmware, or any combination thereof—configured to perform a specified function. For example, a “diagnostic module” may be a physical device or instrument (such as an imaging scanner or a biosensor array) or a software algorithm that processes data from an instrument to yield a diagnostic result. Modules can be distributed or integrated; a module's function can be executed on a local machine or via a remote/cloud-based service, as long as it performs the recited function.

### Compounds and Compositions

### Compounds

Disclosed herein, in certain embodiments, are compounds of Formula (A):

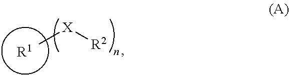

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

As generally defined above R¹ is a polyol, amino acid, or a derivative thereof. In certain embodiments, R¹ is a polyol. A polyol is a compound containing two or more hydroxy (OH) groups. In certain embodiments, R¹ is a polyol which is capable of delivering about 1 g to 50 g or more of ketone bodies to a subject diagnosed with a glucose hypometabolic disorder. In certain embodiments, R¹ is a polyol which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a polyol which is capable of delivering about 1 g to 5 g or more of ketone bodies to a subject diagnosed with a glucose hypometabolic disorder. In certain embodiments, R¹ is a polyol which is capable of delivering about 5 g to 10 g or more of ketone bodies to a subject diagnosed with a glucose hypometabolic disorder. In certain embodiments, R¹ is a polyol which is capable of delivering about 10 g to 15 g or more of ketone bodies to a subject diagnosed with a glucose hypometabolic disorder. In certain embodiments, R¹ is a polyol which is capable of delivering about 15 g to 20 g or more of ketone bodies to a subject diagnosed with a glucose hypometabolic disorder. In certain embodiments, R¹ is a polyol which is capable of delivering about 20 g to 25 g or more of ketone bodies to a subject diagnosed with a glucose hypometabolic disorder. In certain embodiments, R¹ is a polyol which is capable of delivering about 25 g to 30 g or more of ketone bodies to a subject diagnosed with a glucose hypometabolic disorder. In certain embodiments, R¹ is a polyol which is capable of delivering about 30 g to about 35 g or more of ketone bodies to a subject diagnosed with a glucose hypometabolic disorder. In certain embodiments, R¹ is a polyol which is capable of delivering about 35 g to 40 g or more of ketone bodies to a subject diagnosed with a glucose hypometabolic disorder. In certain embodiments, R¹ is a polyol which is capable of delivering about 40 g to 45 g or more of ketone bodies to a subject diagnosed with a glucose hypometabolic disorder. In certain embodiments, R¹ is a polyol which is capable of delivering or about 45 g to 50 g or more of ketone bodies to a subject diagnosed with a glucose hypometabolic disorder.

In certain embodiments, R¹ is a polyol which is capable of delivering about 1 g or more of ketone bodies to a subject diagnosed with a glucose hypometabolic disorder. In certain embodiments, R¹ is a polyol which is capable of delivering about 5 g or more of ketone bodies to a subject diagnosed with a glucose hypometabolic disorder. In certain embodiments, R¹ is a polyol which is capable of delivering about 10 g or more of ketone bodies to a subject diagnosed with a glucose hypometabolic disorder. In certain embodiments, R¹ is a polyol which is capable of delivering about 15 g or more of ketone bodies to a subject diagnosed with a glucose hypometabolic disorder. In certain embodiments, R¹ is a polyol which is capable of delivering about 20 g or more of ketone bodies to a subject diagnosed with a glucose hypometabolic disorder. In certain embodiments, R¹ is a polyol which is capable of delivering about 25 g or more of ketone bodies to a subject diagnosed with a glucose hypometabolic disorder. In certain embodiments, R¹ is a polyol which is capable of delivering about 30 g or more of ketone bodies to a subject diagnosed with a glucose hypometabolic disorder. In certain embodiments, R¹ is a polyol which is capable of delivering about 35 g or more of ketone bodies to a subject diagnosed with a glucose hypometabolic disorder. In certain embodiments, R¹ is a polyol which is capable of delivering about 40 g or more of ketone bodies to a subject diagnosed with a glucose hypometabolic disorder. In certain embodiments, R¹ is a polyol which is capable of delivering or about 45 g or more of ketone bodies to a subject diagnosed with a glucose hypometabolic disorder. In certain embodiments, R¹ is a polyol which is capable of delivering or about 50 g or more of ketone bodies to a subject diagnosed with a glucose hypometabolic disorder.

In certain embodiments, R¹ is a carbohydrate. In certain embodiments, R¹ is a carbohydrate which is capable of delivering about 1 g to 50 g or more of ketone bodies to a subject diagnosed with a glucose hypometabolic disorder. In certain embodiments, R¹ is a carbohydrate which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a carbohydrate which is capable of delivering about 1 g to 5 g or more of ketone bodies to a subject diagnosed with a glucose hypometabolic disorder. In certain embodiments, R¹ is a carbohydrate which is capable of delivering about 5 g to 10 g or more of ketone bodies to a subject diagnosed with a glucose hypometabolic disorder. In certain embodiments, R¹ is a carbohydrate which is capable of delivering about 10 g to 15 g or more of ketone bodies to a subject diagnosed with a glucose hypometabolic disorder. In certain embodiments, R¹ is a carbohydrate which is capable of delivering about 15 g to 20 g or more of ketone bodies to a subject diagnosed with a glucose hypometabolic disorder. In certain embodiments, R¹ is a carbohydrate which is capable of delivering about 20 g to 25 g or more of ketone bodies to a subject diagnosed with a glucose hypometabolic disorder. In certain embodiments, R¹ is a carbohydrate which is capable of delivering about 25 g to 30 g or more of ketone bodies to a subject diagnosed with a glucose hypometabolic disorder. In certain embodiments, R¹ is a carbohydrate which is capable of delivering about 30 g to about 35 g or more of ketone bodies to a subject diagnosed with a glucose hypometabolic disorder. In certain embodiments, R¹ is a carbohydrate which is capable of delivering about 35 g to 40 g or more of ketone bodies to a subject diagnosed with a glucose hypometabolic disorder. In certain embodiments, R¹ is a carbohydrate which is capable of delivering about 40 g to 45 g or more of ketone bodies to a subject diagnosed with a glucose hypometabolic disorder. In certain embodiments, R¹ is a carbohydrate which is capable of delivering or about 45 g to 50 g or more of ketone bodies to a subject diagnosed with a glucose hypometabolic disorder.

In certain embodiments, R¹ is a carbohydrate which is capable of delivering about 1 g or more of ketone bodies to a subject diagnosed with a glucose hypometabolic disorder. In certain embodiments, R¹ is a carbohydrate which is capable of delivering about 5 g or more of ketone bodies to a subject diagnosed with a glucose hypometabolic disorder. In certain embodiments, R¹ is a carbohydrate which is capable of delivering about 10 g or more of ketone bodies to a subject diagnosed with a glucose hypometabolic disorder. In certain embodiments, R¹ is a carbohydrate which is capable of delivering about 15 g or more of ketone bodies to a subject diagnosed with a glucose hypometabolic disorder. In certain embodiments, R¹ is an carbohydrate which is capable of delivering about 20 g or more of ketone bodies to a subject diagnosed with a glucose hypometabolic disorder. In certain embodiments, R¹ is a carbohydrate which is capable of delivering about 25 g or more of ketone bodies to a subject diagnosed with a glucose hypometabolic disorder. In certain embodiments, R¹ is an carbohydrate which is capable of delivering about 30 g or more of ketone bodies to a subject diagnosed with a glucose hypometabolic disorder. In certain embodiments, R¹ is a carbohydrate which is capable of delivering about 35 g or more of ketone bodies to a subject diagnosed with a glucose hypometabolic disorder. In certain embodiments, R¹ is a carbohydrate which is capable of delivering about 40 g or more of ketone bodies to a subject diagnosed with a glucose hypometabolic disorder. In certain embodiments, R¹ is a carbohydrate which is capable of delivering or about 45 g or more of ketone bodies to a subject diagnosed with a glucose hypometabolic disorder. In certain embodiments, R¹ is a carbohydrate which is capable of delivering or about 50 g or more of ketone bodies to a subject diagnosed with a glucose hypometabolic disorder.

In certain embodiments, R¹ is an amino acid or a derivative thereof. In certain embodiments, R¹ is a amino acid or a derivative thereof which is capable of delivering about 1 g to 50 g or more of ketone bodies to a subject diagnosed with a glucose hypometabolic disorder. In certain embodiments, R¹ is a amino acid or a derivative thereof which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a amino acid or a derivative thereof which is capable of delivering about 1 g to 5 g or more of ketone bodies to a subject diagnosed with a glucose hypometabolic disorder. In certain embodiments, R¹ is a amino acid or a derivative thereof which is capable of delivering about 5 g to 10 g or more of ketone bodies to a subject diagnosed with a glucose hypometabolic disorder. In certain embodiments, R¹ is a amino acid or a derivative thereof which is capable of delivering about 10 g to 15 g or more of ketone bodies to a subject diagnosed with a glucose hypometabolic disorder. In certain embodiments, R¹ is a amino acid or a derivative thereof which is capable of delivering about 15 g to 20 g or more of ketone bodies to a subject diagnosed with a glucose hypometabolic disorder. In certain embodiments, R¹ is a amino acid or a derivative thereof which is capable of delivering about 20 g to 25 g or more of ketone bodies to a subject diagnosed with a glucose hypometabolic disorder. In certain embodiments, R¹ is a amino acid or a derivative thereof which is capable of delivering about 25 g to 30 g or more of ketone bodies to a subject diagnosed with a glucose hypometabolic disorder. In certain embodiments, R¹ is a amino acid or a derivative thereof which is capable of delivering about 30 g to about 35 g or more of ketone bodies to a subject diagnosed with a glucose hypometabolic disorder. In certain embodiments, R¹ is a amino acid or a derivative thereof which is capable of delivering about 35 g to 40 g or more of ketone bodies to a subject diagnosed with a glucose hypometabolic disorder. In certain embodiments, R¹ is a amino acid or a derivative thereof which is capable of delivering about 40 g to 45 g or more of ketone bodies to a subject diagnosed with a glucose hypometabolic disorder. In certain embodiments, R¹ is a amino acid or a derivative thereof which is capable of delivering or about 45 g to 50 g or more of ketone bodies to a subject diagnosed with a glucose hypometabolic disorder.

In certain embodiments, R¹ is a amino acid or a derivative thereof which is capable of delivering about 1 g or more of ketone bodies to a subject diagnosed with a glucose hypometabolic disorder. In certain embodiments, R¹ is a amino acid or a derivative thereof which is capable of delivering about 5 g or more of ketone bodies to a subject diagnosed with a glucose hypometabolic disorder. In certain embodiments, R¹ is a amino acid or a derivative thereof which is capable of delivering about 10 g or more of ketone bodies to a subject diagnosed with a glucose hypometabolic disorder. In certain embodiments, R¹ is a amino acid or a derivative thereof which is capable of delivering about 15 g or more of ketone bodies to a subject diagnosed with a glucose hypometabolic disorder. In certain embodiments, R¹ is an amino acid or a derivative thereof which is capable of delivering about 20 g or more of ketone bodies to a subject diagnosed with a glucose hypometabolic disorder. In certain embodiments, R¹ is a amino acid or a derivative thereof which is capable of delivering about 25 g or more of ketone bodies to a subject diagnosed with a glucose hypometabolic disorder. In certain embodiments, R¹ is an amino acid or a derivative thereof which is capable of delivering about 30 g or more of ketone bodies to a subject diagnosed with a glucose hypometabolic disorder. In certain embodiments, R¹ is a amino acid or a derivative thereof which is capable of delivering about 35 g or more of ketone bodies to a subject diagnosed with a glucose hypometabolic disorder. In certain embodiments, R¹ is a amino acid or a derivative thereof which is capable of delivering about 40 g or more of ketone bodies to a subject diagnosed with a glucose hypometabolic disorder. In certain embodiments, R¹ is a amino acid or a derivative thereof which is capable of delivering or about 45 g or more of ketone bodies to a subject diagnosed with a glucose hypometabolic disorder. In certain embodiments, R¹ is a amino acid or a derivative thereof which is capable of delivering or about 50 g or more of ketone bodies to a subject diagnosed with a glucose hypometabolic disorder.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose.

As generally defined above, X is a covalent linker joining selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—. In certain embodiments, X is —O—C(O)—. In certain embodiments, X is —C(O)—O—. In certain embodiments, X is —C(O)—NH—. In certain embodiments, X is —NH—C(O)—. In certain embodiments, X is —C(O)—S—. In certain embodiments, X is —S—C(O)—. In certain embodiments, X is —O—C(O)—O—. In certain embodiments, X is —O—C(O)—NH— or —NH—C(O)—O—. In certain embodiments, X is —O—P(O₂)—O—. In certain embodiments, X is —O—SO₂—O—. In certain embodiments, X is —C═N—. In certain embodiments, X is —C═N—NH—. In certain embodiments, X is —O—. In certain embodiments, X is —NH—. In certain embodiments, X is —S—. In certain embodiments, X is —S—S—. In preferred embodiments, X is —O— or —NH—.

As generally defined above R² is a ketone body moiety selected from β-hydroxybutyryl (BHB), acetoacetyl (AcAc), or a derivative thereof. In certain embodiments, R² is β-hydroxybutyryl (BHB) or a derivative thereof. In certain embodiments, R² is β-hydroxybutyryl (BHB). In certain embodiments, R² is a acetoacetyl (AcAc) or a derivative thereof. In certain embodiments, R² is a acetoacetyl (AcAc). In certain embodiments, R² is

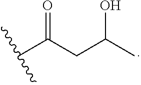 [SMILES: *C(=O)CC(C)O]

In certain embodiments, R² is

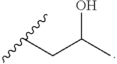 [SMILES: *CC(C)O]

In certain embodiments, R² is

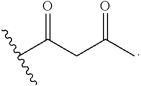 [SMILES: *C(=O)CC(C)=O]

In certain embodiments, R² is

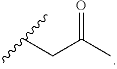 [SMILES: *CC(C)=O]

As generally defined above, n is an integer from 1 to 50. In certain embodiments, n is an integer from 1 to 20. In specific embodiments, n is 1, 2, 3, 4, 5, 6, 7, 8, 9, 10, 11, 12, 13, 14, 15, 16, 17, 18, 19, or 20. In certain embodiments n is 1. In certain embodiments n is 2. In certain embodiments n is 3. In certain embodiments n is 4. In certain embodiments n is 5. In certain embodiments n is 6. In certain embodiments n is 7. In certain embodiments n is 8. In certain embodiments n is 9. In certain embodiments n is 10. In certain embodiments n is 11. In certain embodiments n is 12. In certain embodiments n is 13. In certain embodiments n is 14. In certain embodiments n is 15. In certain embodiments n is 16. In certain embodiments n is 17. In certain embodiments n is 18. In certain embodiments n is 19. In certain embodiments n is 20. In certain embodiments, n is an integer which is equal to the number of hydroxy groups in R¹.

In certain embodiments, the compound is compound of Formula (A):

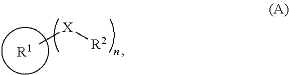

or a pharmaceutically acceptable salt or solvate thereof, wherein
R¹ is selected from the group consisting of trehalose, myo-inositol, D-ribose, inulin, creatine, glycine, carnitine, and taurine;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In certain embodiments, the compound is compound of Formula (A):

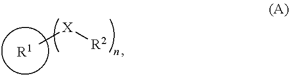

or a pharmaceutically acceptable salt or solvate thereof, wherein
R¹ is selected from the group consisting of trehalose, myo-inositol, D-ribose, inulin, creatine, glycine, carnitine, and taurine;
X is —O— or —NH—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In certain embodiments, the compound is compound of Formula (A):

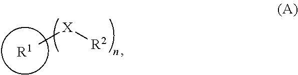

or a pharmaceutically acceptable salt or solvate thereof, wherein
R¹ is trehalose, myo-inositol, or D-ribose;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 8, wherein n represents the total number of ketone body moieties attached to R¹, and wherein n can be less than or equal to the total number of available attachment sites on R¹.

In certain embodiments, the compound is compound of Formula (A):

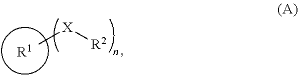

or a pharmaceutically acceptable salt or solvate thereof, wherein
R¹ is trehalose, myo-inositol, or D-ribose;
X is —O—C(O)—;
R² is a ketone body moiety selected from β-hydroxybutyryl, acetoacetyl, or a derivative thereof; and
n is an integer from 1 to 8, wherein n represents the total number of ketone body moieties attached to R¹, and wherein n can be less than or equal to the total number of available attachment sites on R¹.

In certain embodiments, the compound is a compound of Table 1, or a pharmaceutically acceptable salt thereof. It is understood that each of these compounds may exist in partially esterified/amidated forms where not all available hydroxyl or amine groups are conjugated to ketone bodies. Additionally, mixed conjugates are contemplated wherein some attachment sites contain β-hydroxybutyrate moieties while others contain acetoacetate moieties on the same carrier molecule.

TABLE 1
List of compounds.

| Com- | | |
|---|---|---|
| pound | | |
| No. | Name | Structure |
| 1 | Trehalose-BHB Ester (TrehaBHB) | 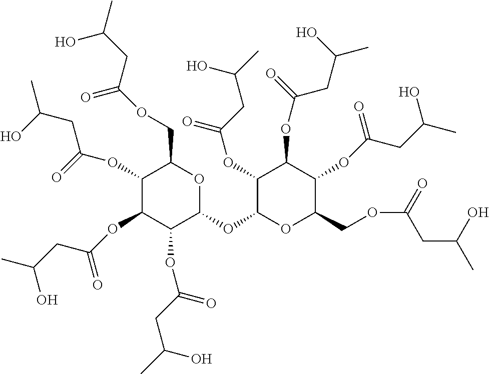 [SMILES: CC(O)CC(=O)OC[C@H]1O[C@H](O[C@H]2O[C@H](COC(=O)CC(C)O)[C@@H](OC(=O)CC(C)O)[C@H](OC(=O)CC(C)O)[C@H]2OC(=O)CC(C)O)[C@H](OC(=O)CC(C)O)[C@@H](OC(=O)CC(C)O)[C@@H]1OC(=O)CC(C)O] |
| 2 | Trehalose- Acetoacetate Ester (TrehaAce) | 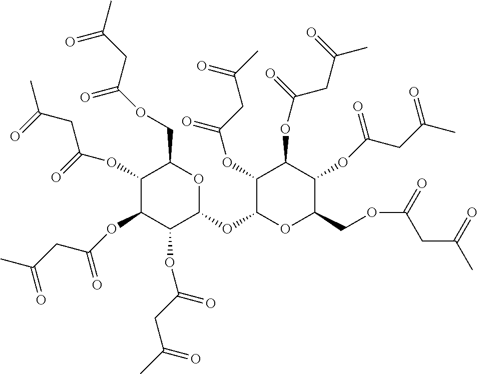 [SMILES: CC(=O)CC(=O)OC[C@H]1O[C@H](O[C@H]2O[C@H](COC(=O)CC(C)=O)[C@@H](OC(=O)CC(C)=O)[C@H](OC(=O)CC(C)=O)[C@H]2OC(=O)CC(C)=O)[C@H](OC(=O)CC(C)=O)[C@@H](OC(=O)CC(C)=O)[C@@H]1OC(=O)CC(C)=O] |
| 3 | Inositol-BHB Ester (InosBHB) | 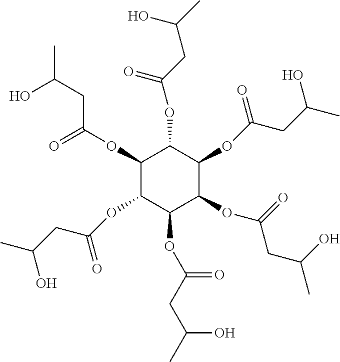 [SMILES: CC(O)CC(=O)O[C@H]1[C@H](OC(=O)CC(C)O)[C@@H](OC(=O)CC(C)O)[C@H](OC(=O)CC(C)O)[C@@H](OC(=O)CC(C)O)[C@H]1OC(=O)CC(C)O] |
| 4 | D-Ribose-BHB Ester (RiboBHB) | 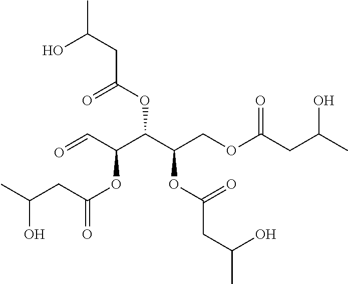 [SMILES: CC(O)CC(=O)OC[C@@H](OC(=O)CC(C)O)[C@@H](OC(=O)CC(C)O)[C@H](C=O)OC(=O)CC(C)O] |
| 5 | Inulin-BHB Ester (FibraBHB) | 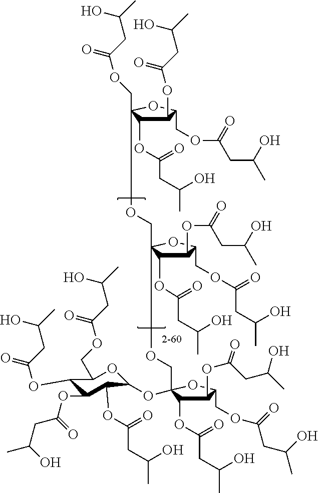 |
| 6 | Creatine-BHB Ester (CreaBHB) | 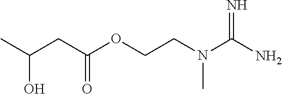 |
| 7 | Glycine- Acetoacetate Amide (GlyAce) | 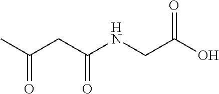 |
| 8 | Carnitine- Acetoacetate Ester (CarnAce) | 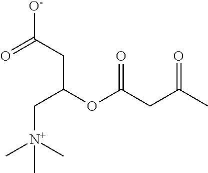 [SMILES: CC(=O)CC(=O)OC(CC(=O)[O-])C[N+](C)(C)C] |
| 9 | Taurine- Acetoacetate Amide (TaurAce) | 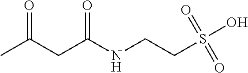 |
| 10 | Inositol- Acetoacetate Ester (InosAce) | 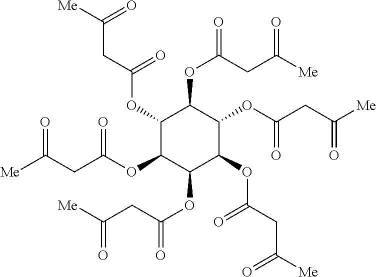 [SMILES: CC(=O)CC(=O)O[C@H]1[C@H](OC(=O)CC(C)=O)[C@@H](OC(=O)CC(C)=O)[C@H](OC(=O)CC(C)=O)[C@@H](OC(=O)CC(C)=O)[C@H]1OC(=O)CC(C)=O] |
| 11 | Glycerol- Acetoacetate Ester (GlycAce) | 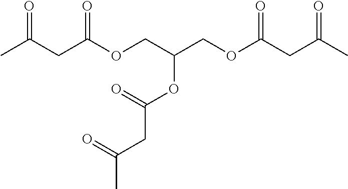 [SMILES: CC(=O)CC(=O)OCC(COC(=O)CC(C)=O)OC(=O)CC(C)=O] |
| 12 | Allulose- Acetoacetate Ester (AllAce) | 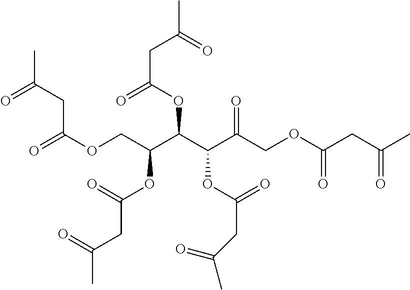 [SMILES: CC(=O)CC(=O)OCC(=O)[C@H](OC(=O)CC(C)=O)[C@H](OC(=O)CC(C)=O)[C@H](COC(=O)CC(C)=O)OC(=O)CC(C)=O] |
| 13 | Aspartic Acid- Acetoacetate Ester (AspAce) | 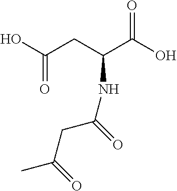 |
| 14 | Glycerol-BHB Ester (GlycBHB) | 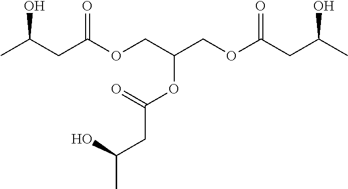 [SMILES: C[C@H](O)CC(=O)OCC(COC(=O)C[C@@H](C)O)OC(=O)C[C@@H](C)O] |
| 15 | Carnosine-BHB Ester (CarnosBHB) | 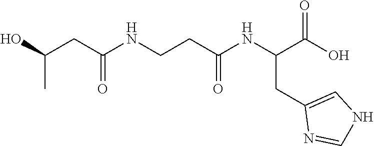 [SMILES: C[C@@H](O)CC(=O)NCCC(=O)NC(Cc1c[nH]cn1)C(=O)O] |

The compounds of Formula (A) described herein offer certain advantages over simpler ketone supplements (such as monomeric ketone esters or salts). By attaching multiple ketone body moieties (R²) to a single polyol backbone (R¹), a single compound can deliver a higher payload of ketone equivalents per molecule, potentially increasing the efficiency of raising blood ketone levels. Additionally, the use of a polyol backbone can impart favorable pharmacokinetic properties—for instance, as the conjugate is metabolized, it may release ketone bodies gradually, acting as a built-in sustained-release mechanism. This could help maintain therapeutically relevant ketone concentrations over a longer duration compared to a rapid spike from a simple ketone salt. In some embodiments, such multi-ketone conjugates may also improve tolerability or palatability: by distributing the ketone load across a larger molecule, peak gastrointestinal irritation or the strong taste associated with free ketones or salts might be reduced. While the invention is not bound by theory, these potential benefits illustrate the innovative nature of the polyol-based ketone conjugates, which go beyond conventional monomeric ketone compounds.

### Compositions

Another aspect of the invention provides compositions comprising one or more compounds of Formula (A). In certain embodiments, the comprises at least two compounds from Table 1.

In certain embodiments, the composition comprises Compound 1 and Compound 2. In certain embodiments, the composition comprises Compound 1 and Compound 3. In certain embodiments, the composition comprises Compound 1 and Compound 4. In certain embodiments, the composition comprises Compound 1 and Compound 5. In certain embodiments, the composition comprises Compound 1 and Compound 6. In certain embodiments, the composition comprises Compound 1 and Compound 7. In certain embodiments, the composition comprises Compound 1 and Compound 8. In certain embodiments, the composition comprises Compound 1 and Compound 9. In certain embodiments, the composition comprises Compound 1 and Compound 10. In certain embodiments, the composition comprises Compound 1 and Compound 11. In certain embodiments, the composition comprises Compound 1 and Compound 12. In certain embodiments, the composition comprises Compound 1 and Compound 13. In certain embodiments, the composition comprises Compound 1 and Compound 14. In certain embodiments, the composition comprises Compound 1 and Compound 15.

In certain embodiments, the composition comprises Compound 2 and Compound 3. In certain embodiments, the composition comprises Compound 2 and Compound 4. In certain embodiments, the composition comprises Compound 2 and Compound 5. In certain embodiments, the composition comprises Compound 2 and Compound 6. In certain embodiments, the composition comprises Compound 2 and Compound 7. In certain embodiments, the composition comprises Compound 2 and Compound 8. In certain embodiments, the composition comprises Compound 2 and Compound 9. In certain embodiments, the composition comprises Compound 2 and Compound 10. In certain embodiments, the composition comprises Compound 2 and Compound 11. In certain embodiments, the composition comprises Compound 2 and Compound 12. In certain embodiments, the composition comprises Compound 2 and Compound 13. In certain embodiments, the composition comprises Compound 2 and Compound 14. In certain embodiments, the composition comprises Compound 2 and Compound 15.

In certain embodiments, the composition comprises Compound 3 and Compound 4. In certain embodiments, the composition comprises Compound 3 and Compound 5. In certain embodiments, the composition comprises Compound 3 and Compound 6. In certain embodiments, the composition comprises Compound 3 and Compound 7. In certain embodiments, the composition comprises Compound 3 and Compound 8. In certain embodiments, the composition comprises Compound 3 and Compound 9. In certain embodiments, the composition comprises Compound 3 and Compound 10. In certain embodiments, the composition comprises Compound 3 and Compound 11. In certain embodiments, the composition comprises Compound 3 and Compound 12. In certain embodiments, the composition comprises Compound 3 and Compound 13. In certain embodiments, the composition comprises Compound 3 and Compound 14. In certain embodiments, the composition comprises Compound 3 and Compound 15.

In certain embodiments, the composition comprises Compound 4 and Compound 5. In certain embodiments, the composition comprises Compound 4 and Compound 6. In certain embodiments, the composition comprises Compound 4 and Compound 7. In certain embodiments, the composition comprises Compound 4 and Compound 8. In certain embodiments, the composition comprises Compound 4 and Compound 9. In certain embodiments, the composition comprises Compound 4 and Compound 10. In certain embodiments, the composition comprises Compound 4 and Compound 11. In certain embodiments, the composition comprises Compound 4 and Compound 12. In certain embodiments, the composition comprises Compound 4 and Compound 13. In certain embodiments, the composition comprises Compound 4 and Compound 14. In certain embodiments, the composition comprises Compound 4 and Compound 15.

In certain embodiments, the composition comprises Compound 5 and Compound 6. In certain embodiments, the composition comprises Compound 5 and Compound 7. In certain embodiments, the composition comprises Compound 5 and Compound 8. In certain embodiments, the composition comprises Compound 5 and Compound 9. In certain embodiments, the composition comprises Compound 5 and Compound 10. In certain embodiments, the composition comprises Compound 5 and Compound 11. In certain embodiments, the composition comprises Compound 5 and Compound 12. In certain embodiments, the composition comprises Compound 5 and Compound 13. In certain embodiments, the composition comprises Compound 5 and Compound 14. In certain embodiments, the composition comprises Compound 5 and Compound 15.

In certain embodiments, the composition comprises Compound 6 and Compound 7. In certain embodiments, the composition comprises Compound 6 and Compound 8. In certain embodiments, the composition comprises Compound 6 and Compound 9. In certain embodiments, the composition comprises Compound 6 and Compound 10. In certain embodiments, the composition comprises Compound 6 and Compound 11. In certain embodiments, the composition comprises Compound 6 and Compound 12. In certain embodiments, the composition comprises Compound 6 and Compound 13. In certain embodiments, the composition comprises Compound 6 and Compound 14. In certain embodiments, the composition comprises Compound 6 and Compound 15.

In certain embodiments, the composition comprises Compound 7 and Compound 8. In certain embodiments, the composition comprises Compound 7 and Compound 9. In certain embodiments, the composition comprises Compound 7 and Compound 10. In certain embodiments, the composition comprises Compound 7 and Compound 11. In certain embodiments, the composition comprises Compound 7 and Compound 12. In certain embodiments, the composition comprises Compound 7 and Compound 13. In certain embodiments, the composition comprises Compound 7 and Compound 14. In certain embodiments, the composition comprises Compound 7 and Compound 15.

In certain embodiments, the composition comprises Compound 8 and Compound 9. In certain embodiments, the composition comprises Compound 8 and Compound 10. In certain embodiments, the composition comprises Compound 8 and Compound 11. In certain embodiments, the composition comprises Compound 8 and Compound 12. In certain embodiments, the composition comprises Compound 8 and Compound 13. In certain embodiments, the composition comprises Compound 8 and Compound 14. In certain embodiments, the composition comprises Compound 8 and Compound 15.

In certain embodiments, the composition comprises Compound 9 and Compound 10. In certain embodiments, the composition comprises Compound 9 and Compound 11. In certain embodiments, the composition comprises Compound 9 and Compound 12. In certain embodiments, the composition comprises Compound 9 and Compound 13. In certain embodiments, the composition comprises Compound 9 and Compound 14. In certain embodiments, the composition comprises Compound 9 and Compound 15.

In certain embodiments, the composition comprises Compound 10 and Compound 11. In certain embodiments, the composition comprises Compound 10 and Compound 12. In certain embodiments, the composition comprises Compound 10 and Compound 13. In certain embodiments, the composition comprises Compound 10 and Compound 14. In certain embodiments, the composition comprises Compound 10 and Compound 15.

In certain embodiments, the composition comprises Compound 11 and Compound 12. In certain embodiments, the composition comprises Compound 11 and Compound 13. In certain embodiments, the composition comprises Compound 11 and Compound 14. In certain embodiments, the composition comprises Compound 11 and Compound 15.

In certain embodiments, the composition comprises Compound 12 and Compound 13. In certain embodiments, the composition comprises Compound 12 and Compound 14. In certain embodiments, the composition comprises Compound 12 and Compound 15.

In certain embodiments, the composition comprises Compound 13 and Compound 14. In certain embodiments, the composition comprises Compound 13 and Compound 15.

In certain embodiments, the composition comprises Compound 14 and Compound 15.

In certain embodiments, the composition comprises at least two compounds of Formula (A). In certain embodiments, each compound of Formula (A) has the same R¹ and X.

As generally defined above R¹ is a polyol, amino acid, or a derivative thereof. In certain embodiments, R¹ is a polyol. A polyol is a compound containing two or more hydroxy (OH) groups.

In certain embodiments, R¹ is a polyol which is capable of delivering about 1 g to 50 g or more of ketone bodies to a subject diagnosed with a glucose hypometabolic disorder. In certain embodiments, R¹ is a polyol which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a polyol which is capable of delivering about 1 g to 5 g or more of ketone bodies to a subject diagnosed with a glucose hypometabolic disorder. In certain embodiments, R¹ is a polyol which is capable of delivering about 5 g to 10 g or more of ketone bodies to a subject diagnosed with a glucose hypometabolic disorder. In certain embodiments, R¹ is a polyol which is capable of delivering about 10 g to 15 g or more of ketone bodies to a subject diagnosed with a glucose hypometabolic disorder. In certain embodiments, R¹ is a polyol which is capable of delivering about 15 g to 20 g or more of ketone bodies to a subject diagnosed with a glucose hypometabolic disorder. In certain embodiments, R¹ is a polyol which is capable of delivering about 20 g to 25 g or more of ketone bodies to a subject diagnosed with a glucose hypometabolic disorder. In certain embodiments, R¹ is a polyol which is capable of delivering about 25 g to 30 g or more of ketone bodies to a subject diagnosed with a glucose hypometabolic disorder. In certain embodiments, R¹ is a polyol which is capable of delivering about 30 g to about 35 g or more of ketone bodies to a subject diagnosed with a glucose hypometabolic disorder. In certain embodiments, R¹ is a polyol which is capable of delivering about 35 g to 40 g or more of ketone bodies to a subject diagnosed with a glucose hypometabolic disorder. In certain embodiments, R¹ is a polyol which is capable of delivering about 40 g to 45 g or more of ketone bodies to a subject diagnosed with a glucose hypometabolic disorder. In certain embodiments, R¹ is a polyol which is capable of delivering or about 45 g to 50 g or more of ketone bodies to a subject diagnosed with a glucose hypometabolic disorder.

In certain embodiments, R¹ is a polyol which is capable of delivering about 1 g or more of ketone bodies to a subject diagnosed with a glucose hypometabolic disorder. In certain embodiments, R¹ is a polyol which is capable of delivering about 5 g or more of ketone bodies to a subject diagnosed with a glucose hypometabolic disorder. In certain embodiments, R¹ is a polyol which is capable of delivering about 10 g or more of ketone bodies to a subject diagnosed with a glucose hypometabolic disorder. In certain embodiments, R¹ is a polyol which is capable of delivering about 15 g or more of ketone bodies to a subject diagnosed with a glucose hypometabolic disorder. In certain embodiments, R¹ is a polyol which is capable of delivering about 20 g or more of ketone bodies to a subject diagnosed with a glucose hypometabolic disorder. In certain embodiments, R¹ is a polyol which is capable of delivering about 25 g or more of ketone bodies to a subject diagnosed with a glucose hypometabolic disorder. In certain embodiments, R¹ is a polyol which is capable of delivering about 30 g or more of ketone bodies to a subject diagnosed with a glucose hypometabolic disorder. In certain embodiments, R¹ is a polyol which is capable of delivering about 35 g or more of ketone bodies to a subject diagnosed with a glucose hypometabolic disorder. In certain embodiments, R¹ is a polyol which is capable of delivering about 40 g or more of ketone bodies to a subject diagnosed with a glucose hypometabolic disorder. In certain embodiments, R¹ is a polyol which is capable of delivering or about 45 g or more of ketone bodies to a subject diagnosed with a glucose hypometabolic disorder. In certain embodiments, R¹ is a polyol which is capable of delivering or about 50 g or more of ketone bodies to a subject diagnosed with a glucose hypometabolic disorder.

In certain embodiments, R¹ is a carbohydrate. In certain embodiments, R¹ is a carbohydrate which is capable of delivering about 1 g to 50 g or more of ketone bodies to a subject diagnosed with a glucose hypometabolic disorder. In certain embodiments, R¹ is a carbohydrate which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a carbohydrate which is capable of delivering about 1 g to 5 g or more of ketone bodies to a subject diagnosed with a glucose hypometabolic disorder. In certain embodiments, R¹ is a carbohydrate which is capable of delivering about 5 g to 10 g or more of ketone bodies to a subject diagnosed with a glucose hypometabolic disorder. In certain embodiments, R¹ is a carbohydrate which is capable of delivering about 10 g to 15 g or more of ketone bodies to a subject diagnosed with a glucose hypometabolic disorder. In certain embodiments, R¹ is a carbohydrate which is capable of delivering about 15 g to 20 g or more of ketone bodies to a subject diagnosed with a glucose hypometabolic disorder. In certain embodiments, R¹ is a carbohydrate which is capable of delivering about 20 g to 25 g or more of ketone bodies to a subject diagnosed with a glucose hypometabolic disorder. In certain embodiments, R¹ is a carbohydrate which is capable of delivering about 25 g to 30 g or more of ketone bodies to a subject diagnosed with a glucose hypometabolic disorder. In certain embodiments, R¹ is a carbohydrate which is capable of delivering about 30 g to about 35 g or more of ketone bodies to a subject diagnosed with a glucose hypometabolic disorder. In certain embodiments, R¹ is a carbohydrate which is capable of delivering about 35 g to 40 g or more of ketone bodies to a subject diagnosed with a glucose hypometabolic disorder. In certain embodiments, R¹ is a carbohydrate which is capable of delivering about 40 g to 45 g or more of ketone bodies to a subject diagnosed with a glucose hypometabolic disorder. In certain embodiments, R¹ is a carbohydrate which is capable of delivering or about 45 g to 50 g or more of ketone bodies to a subject diagnosed with a glucose hypometabolic disorder.

In certain embodiments, R¹ is a carbohydrate which is capable of delivering about 1 g or more of ketone bodies to a subject diagnosed with a glucose hypometabolic disorder. In certain embodiments, R¹ is a carbohydrate which is capable of delivering about 5 g or more of ketone bodies to a subject diagnosed with a glucose hypometabolic disorder. In certain embodiments, R¹ is a carbohydrate which is capable of delivering about 10 g or more of ketone bodies to a subject diagnosed with a glucose hypometabolic disorder. In certain embodiments, R¹ is a carbohydrate which is capable of delivering about 15 g or more of ketone bodies to a subject diagnosed with a glucose hypometabolic disorder. In certain embodiments, R¹ is an carbohydrate which is capable of delivering about 20 g or more of ketone bodies to a subject diagnosed with a glucose hypometabolic disorder. In certain embodiments, R¹ is a carbohydrate which is capable of delivering about 25 g or more of ketone bodies to a subject diagnosed with a glucose hypometabolic disorder. In certain embodiments, R¹ is an carbohydrate which is capable of delivering about 30 g or more of ketone bodies to a subject diagnosed with a glucose hypometabolic disorder. In certain embodiments, R¹ is a carbohydrate which is capable of delivering about 35 g or more of ketone bodies to a subject diagnosed with a glucose hypometabolic disorder. In certain embodiments, R¹ is a carbohydrate which is capable of delivering about 40 g or more of ketone bodies to a subject diagnosed with a glucose hypometabolic disorder. In certain embodiments, R¹ is a carbohydrate which is capable of delivering or about 45 g or more of ketone bodies to a subject diagnosed with a glucose hypometabolic disorder. In certain embodiments, R¹ is a carbohydrate which is capable of delivering or about 50 g or more of ketone bodies to a subject diagnosed with a glucose hypometabolic disorder.

In certain embodiments, R¹ is an amino acid or a derivative thereof. In certain embodiments, R¹ is a amino acid or a derivative thereof which is capable of delivering about 1 g to 50 g or more of ketone bodies to a subject diagnosed with a glucose hypometabolic disorder. In certain embodiments, R¹ is a amino acid or a derivative thereof which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a amino acid or a derivative thereof which is capable of delivering about 1 g to 5 g or more of ketone bodies to a subject diagnosed with a glucose hypometabolic disorder. In certain embodiments, R¹ is a amino acid or a derivative thereof which is capable of delivering about 5 g to 10 g or more of ketone bodies to a subject diagnosed with a glucose hypometabolic disorder. In certain embodiments, R¹ is a amino acid or a derivative thereof which is capable of delivering about 10 g to 15 g or more of ketone bodies to a subject diagnosed with a glucose hypometabolic disorder. In certain embodiments, R¹ is a amino acid or a derivative thereof which is capable of delivering about 15 g to 20 g or more of ketone bodies to a subject diagnosed with a glucose hypometabolic disorder. In certain embodiments, R¹ is a amino acid or a derivative thereof which is capable of delivering about 20 g to 25 g or more of ketone bodies to a subject diagnosed with a glucose hypometabolic disorder. In certain embodiments, R¹ is a amino acid or a derivative thereof which is capable of delivering about 25 g to 30 g or more of ketone bodies to a subject diagnosed with a glucose hypometabolic disorder. In certain embodiments, R¹ is a amino acid or a derivative thereof which is capable of delivering about 30 g to about 35 g or more of ketone bodies to a subject diagnosed with a glucose hypometabolic disorder. In certain embodiments, R¹ is a amino acid or a derivative thereof which is capable of delivering about 35 g to 40 g or more of ketone bodies to a subject diagnosed with a glucose hypometabolic disorder. In certain embodiments, R¹ is a amino acid or a derivative thereof which is capable of delivering about 40 g to 45 g or more of ketone bodies to a subject diagnosed with a glucose hypometabolic disorder. In certain embodiments, R¹ is a amino acid or a derivative thereof which is capable of delivering or about 45 g to 50 g or more of ketone bodies to a subject diagnosed with a glucose hypometabolic disorder.

In certain embodiments, R¹ is a amino acid or a derivative thereof which is capable of delivering about 1 g or more of ketone bodies to a subject diagnosed with a glucose hypometabolic disorder. In certain embodiments, R¹ is a amino acid or a derivative thereof which is capable of delivering about 5 g or more of ketone bodies to a subject diagnosed with a glucose hypometabolic disorder. In certain embodiments, R¹ is a amino acid or a derivative thereof which is capable of delivering about 10 g or more of ketone bodies to a subject diagnosed with a glucose hypometabolic disorder. In certain embodiments, R¹ is a amino acid or a derivative thereof which is capable of delivering about 15 g or more of ketone bodies to a subject diagnosed with a glucose hypometabolic disorder. In certain embodiments, R¹ is an amino acid or a derivative thereof which is capable of delivering about 20 g or more of ketone bodies to a subject diagnosed with a glucose hypometabolic disorder. In certain embodiments, R¹ is a amino acid or a derivative thereof which is capable of delivering about 25 g or more of ketone bodies to a subject diagnosed with a glucose hypometabolic disorder. In certain embodiments, R¹ is an amino acid or a derivative thereof which is capable of delivering about 30 g or more of ketone bodies to a subject diagnosed with a glucose hypometabolic disorder. In certain embodiments, R¹ is a amino acid or a derivative thereof which is capable of delivering about 35 g or more of ketone bodies to a subject diagnosed with a glucose hypometabolic disorder. In certain embodiments, R¹ is a amino acid or a derivative thereof which is capable of delivering about 40 g or more of ketone bodies to a subject diagnosed with a glucose hypometabolic disorder. In certain embodiments, R¹ is a amino acid or a derivative thereof which is capable of delivering or about 45 g or more of ketone bodies to a subject diagnosed with a glucose hypometabolic disorder. In certain embodiments, R¹ is a amino acid or a derivative thereof which is capable of delivering or about 50 g or more of ketone bodies to a subject diagnosed with a glucose hypometabolic disorder.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

As generally defined above, X is a covalent linker joining selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—. In certain embodiments, X is —O—C(O)—. In certain embodiments, X is —C(O)—O—. In certain embodiments, X is —C(O)—NH—. In certain embodiments, X is —NH—C(O)—. In certain embodiments, X is —C(O)—S—. In certain embodiments, X is —S—C(O)—. In certain embodiments, X is —O—C(O)—O—. In certain embodiments, X is —O—C(O)—NH— or —NH—C(O)—O—. In certain embodiments, X is —O—P(O₂)—O—. In certain embodiments, X is —O—SO₂—O—. In certain embodiments, X is —C═N—. In certain embodiments, X is —C═N—NH—. In certain embodiments, X is —O—. In certain embodiments, X is —NH—. In certain embodiments, X is —S—. In certain embodiments, X is —S—S—. In preferred embodiments, X is —O— or —NH—.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

In certain embodiments, the composition comprises at least two unique compounds of Formula (A). In certain embodiments, the composition comprises one unique compound of Formula (A) is:

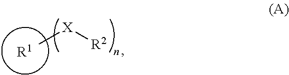

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl or a derivative thereof, and
n is an integer from 1 to 50, wherein n represents the total number of ketone body moieties attached to R¹, and wherein n can be less than or equal to the total number of available attachment sites on R¹, and
wherein the other unique compound of Formula (A) is:

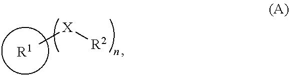

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from acetoacetyl (AcAc) or a derivative thereof; and
n is an integer from 1 to 50, wherein n represents the total number of ketone body moieties attached to R¹, and wherein n can be less than or equal to the total number of available attachment sites on R¹.

The compounds of Formula (A) may be formulated as pharmaceutical compositions by combining the compound with pharmaceutically acceptable carriers, excipients, and/or diluents. Such compositions may be formulated for oral, enteral, parenteral, or intravenous administration.

For oral administration, the compounds may be formulated as tablets, capsules, powders, solutions, suspensions, or emulsions. For parenteral administration, the compounds may be formulated as solutions, suspensions, or emulsions in aqueous or oil vehicles.

### Therapeutic Applications and Advantages

The compounds and compositions of the present disclosure provide significant therapeutic advantages for treating glucose hypometabolic disorders:

Enhanced Bioavailability: By conjugating ketone bodies to carrier molecules via stable linkers (e.g., ester or amide bonds), the compounds and compositions exhibit improved absorption, extended circulation time, and enhanced tissue penetration compared to free ketone bodies.

Increased Ketone Payload: In certain embodiments, the multi-esterification design allows delivery of multiple ketone moieties per carrier molecule, maximizing the therapeutic effect while minimizing excipient load.

Targeted Delivery: Specific carrier molecules can preferentially direct ketones to tissues most affected by glucose hypometabolism. For example, trehalose-based conjugates may have enhanced blood-brain barrier penetration for neurological conditions.

Controlled Release: The linkers (e.g., the ester or amide bonds) are enzymatically hydrolyzed in vivo, providing sustained release of ketone bodies over time rather than the sharp peaks and troughs seen with direct ketone salt administration.

Synergistic Effects: The carrier molecules themselves often have complementary therapeutic effects. For example, inositol supports membrane stabilization and signaling, while creatine enhances cellular energy reserves.

Reduced Side Effects: The conjugate formulations minimize gastrointestinal distress and electrolyte imbalances commonly associated with ketone salt administration.

Dosing Convenience: The stable solid formulations allow for convenient oral dosing, improving patient compliance compared to liquid formulations or dietary interventions.

### Mechanisms of Action

The compounds and compositions of the present disclosure work through multiple complementary mechanisms:

Metabolic Bypass: The released ketone bodies provide an alternative energy substrate that bypasses the impaired glucose metabolism pathways seen in conditions like Alzheimer's disease, Parkinson's disease, and heart failure.

Enhanced Energetic Efficiency: Ketone bodies produce more ATP per oxygen molecule compared to glucose (2.5 vs 2.25 ATP/O), providing energetic advantages in oxygen-limited or mitochondrially compromised tissues.

Reduced Oxidative Stress: Ketone metabolism produces fewer reactive oxygen species than glucose or fatty acid oxidation, reducing oxidative damage in affected tissues.

Signaling Effects: Beyond serving as energy substrates, ketone bodies (particularly (3-hydroxybutyrate) activate beneficial signaling pathways, including those related to mitochondrial biogenesis, antioxidant defense, and epigenetic regulation through histone deacetylase inhibition.

Neuroprotection: In neurodegenerative conditions, ketone bodies provide neuroprotection through multiple mechanisms, including reduced excitotoxicity, enhanced GABA signaling, and stabilization of neuronal membrane potentials.

Cardiac Protection: In heart failure, ketone bodies improve cardiac efficiency, reduce fibrosis, and enhance calcium handling in cardiomyocytes.

### Methods

Disclosed herein, in certain embodiments, are methods of treating a subject diagnosed with a glucose hypometabolic disorder (e.g., Alzheimer's disease, Parkinson's disease, or heart failure) comprising administering a therapeutically effective amount of a compound of Formula (A):

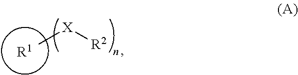

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

Also disclosed herein, in certain embodiments, are methods for diagnosing and quantifying glucose hypometabolism in a subject comprising administering a therapeutically effective amount of a compound of Formula (A):

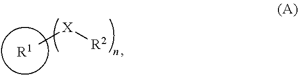

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.n some embodiments, the compound of Formula (A) is a compound in Table 1. In some embodiments, the compound is selected from the group consisting of:

In another aspect, provided herein is a method for treating Alzheimer's disease, the method comprising administering to a subject a therapeutically effective amount of a compound of Formula (A):

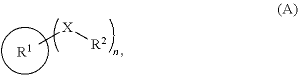

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

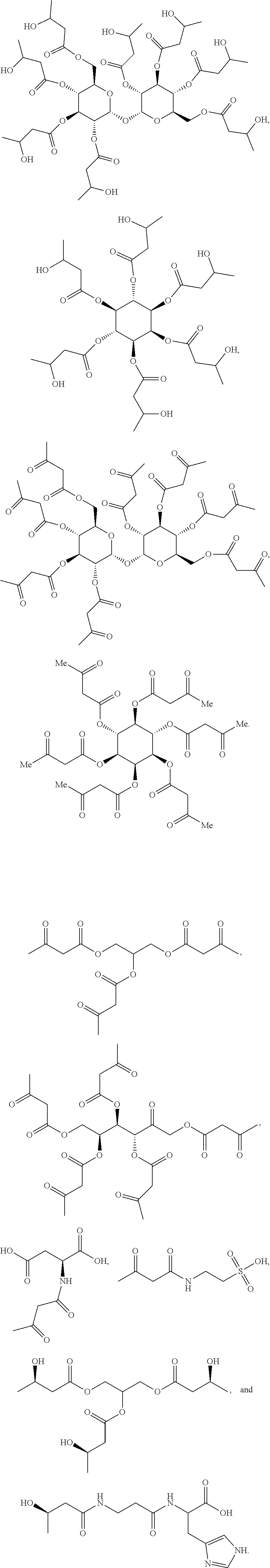

In another aspect, provided herein is a method for treating Parkinson's disease, the method comprising administering to a subject a therapeutically effective amount of a compound of Formula (A):

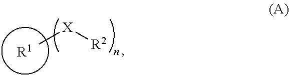

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

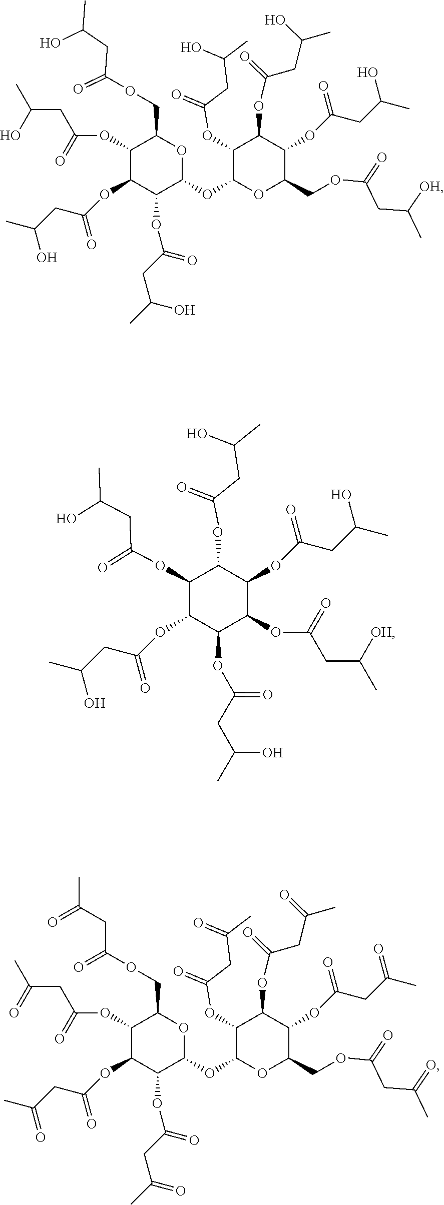

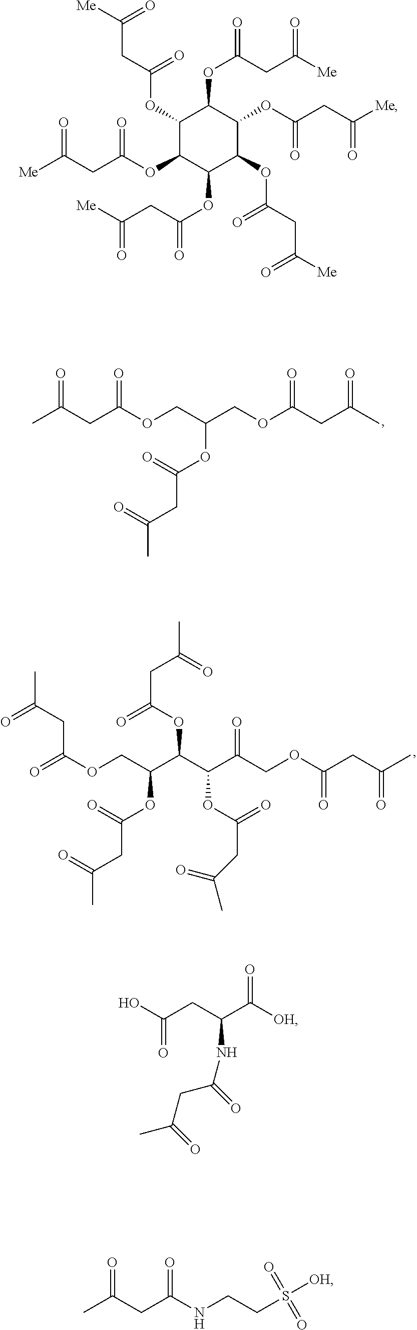

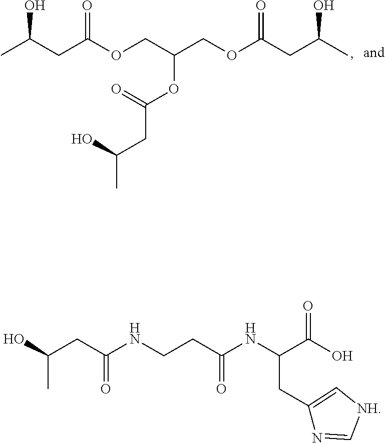 [SMILES: C[C@H](O)CC(=O)OCC(COC(=O)C[C@@H](C)O)OC(=O)C[C@@H](C)O ; C[C@@H](O)CC(=O)NCCC(=O)NC(Cc1c[nH]cn1)C(=O)O]

In another aspect, provided herein is a method for treating Huntington's disease, the method comprising administering to a subject a therapeutically effective amount of a compound of Formula (A):

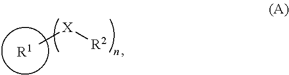

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

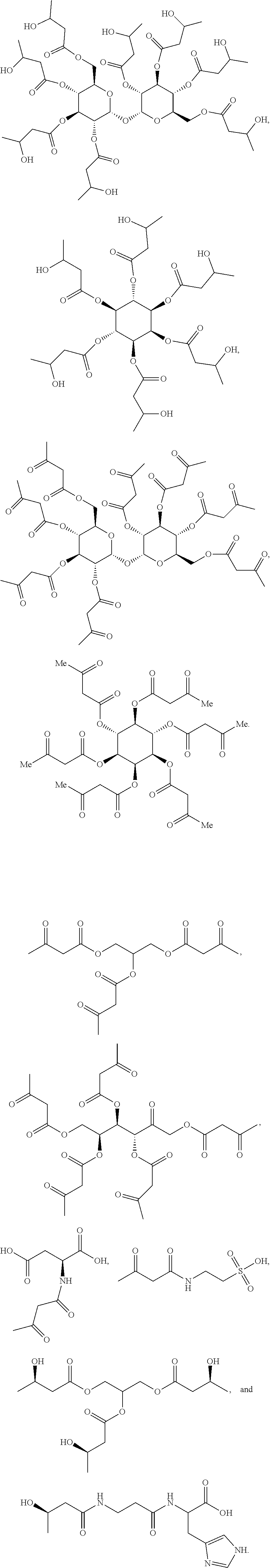

In another aspect, provided herein is a method for treating heart failure, the method comprising administering to a subject a therapeutically effective amount of a compound of Formula (A):

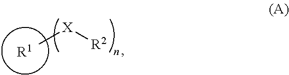

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof, and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

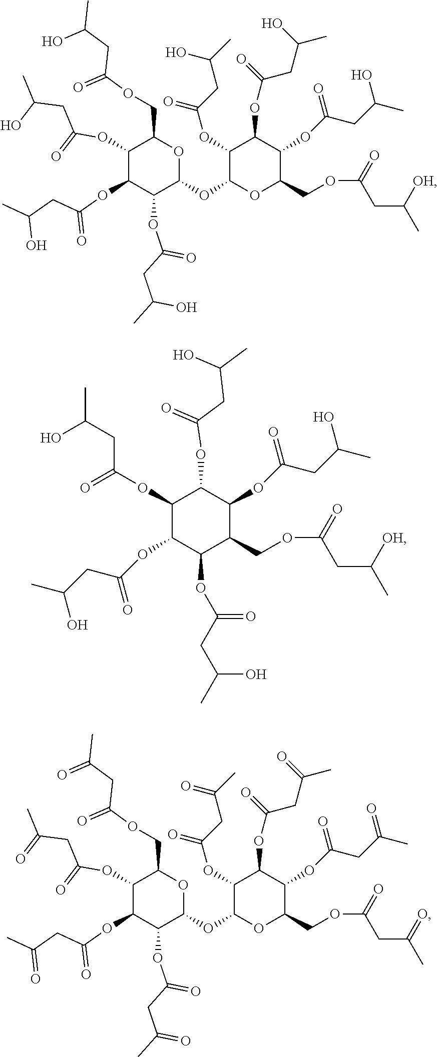 [SMILES: CC(O)CC(=O)OC[C@H]1O[C@H](O[C@H]2O[C@H](COC(=O)CC(C)O)[C@@H](OC(=O)CC(C)O)[C@H](OC(=O)CC(C)O)[C@H]2OC(=O)CC(C)O)[C@H](OC(=O)CC(C)O)[C@@H](OC(=O)CC(C)O)[C@@H]1OC(=O)CC(C)O ; CC(O)CC(=O)OC[C@H]1[C@@H](OC(=O)CC(C)O)[C@H](OC(=O)CC(C)O)[C@@H](OC(=O)CC(C)O)[C@H](OC(=O)CC(C)O)[C@H]1OC(=O)CC(C)O ; CC(=O)CC(=O)OC[C@H]1O[C@H](O[C@H]2O[C@H](COC(=O)CC(C)=O)[C@@H](OC(=O)CC(C)=O)[C@H](OC(=O)CC(C)=O)[C@H]2OC(=O)CC(C)=O)[C@H](OC(=O)CC(C)=O)[C@@H](OC(=O)CC(C)=O)[C@@H]1OC(=O)CC(C)=O]

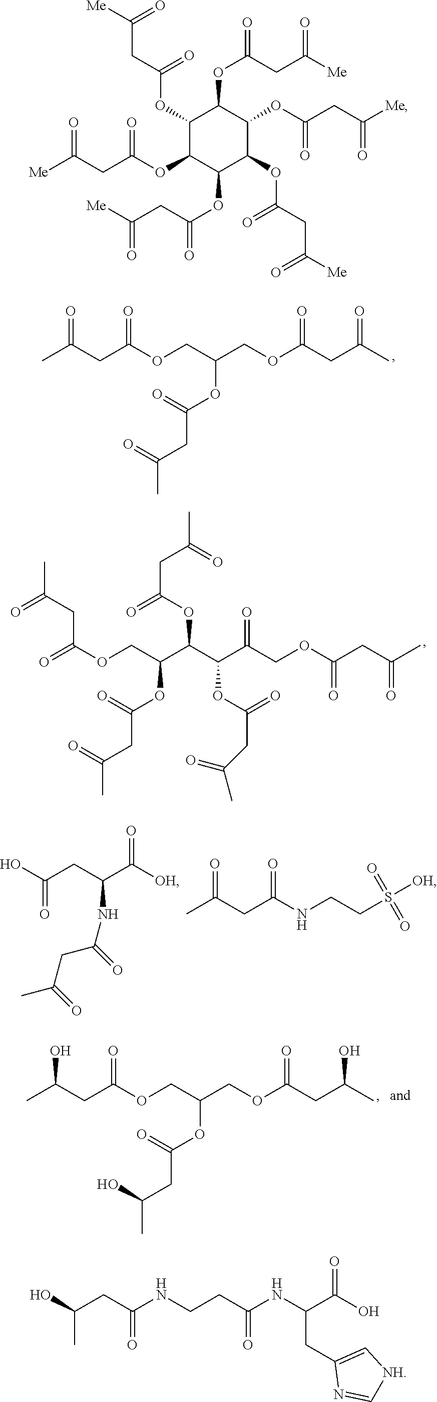

In another aspect, provided herein is a method for treating stroke, the method comprising administering to a subject a therapeutically effective amount of a compound of Formula (A):

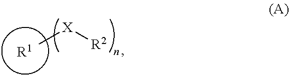

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

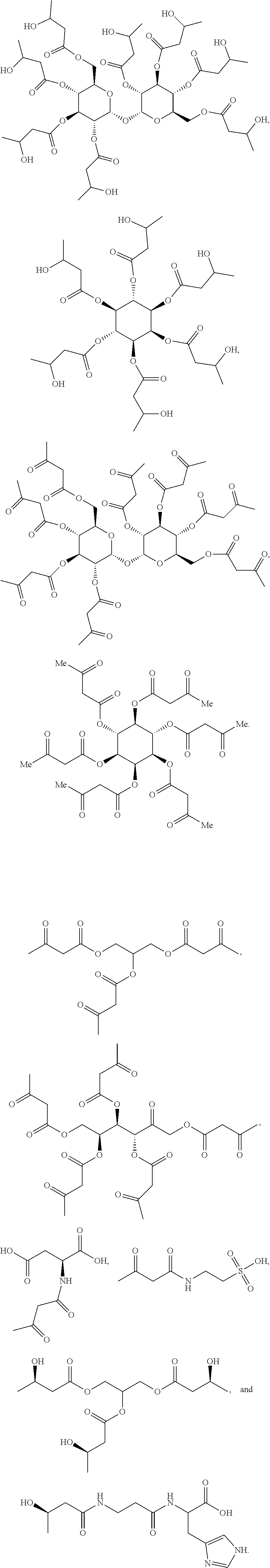

In another aspect, provided herein is a method for treating spinal cord injury, the method comprising administering to a subject a therapeutically effective amount of a compound of Formula (A):

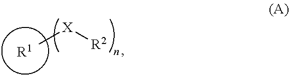

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

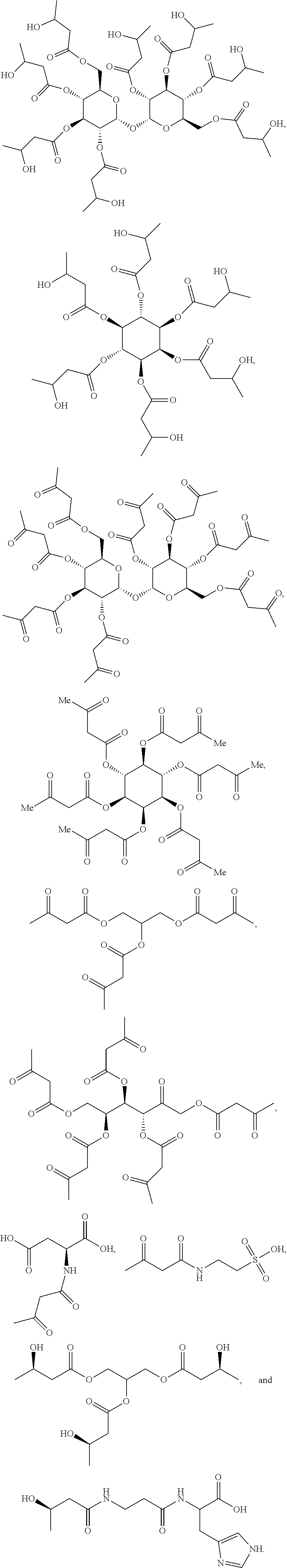

In another aspect, provided herein is a method for treating type 2 diabetes, the method comprising administering to a subject a therapeutically effective amount of a compound of Formula (A):

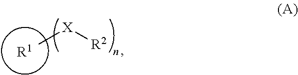

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

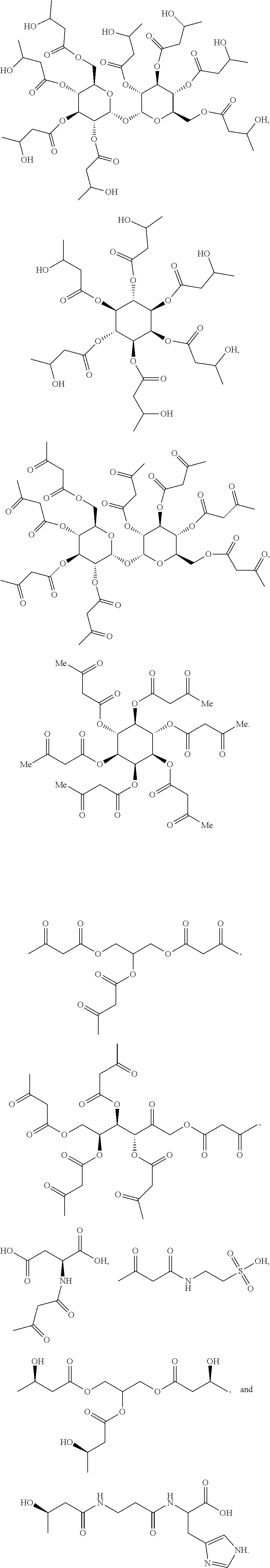

In another aspect, provided herein is a method for treating obesity, the method comprising administering to a subject a therapeutically effective amount of a compound of Formula (A):

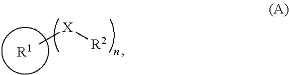

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

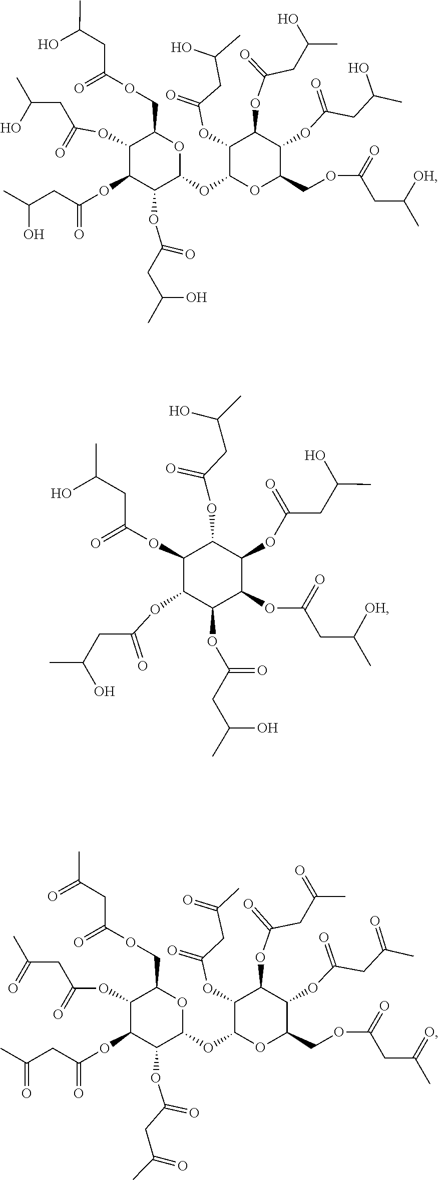

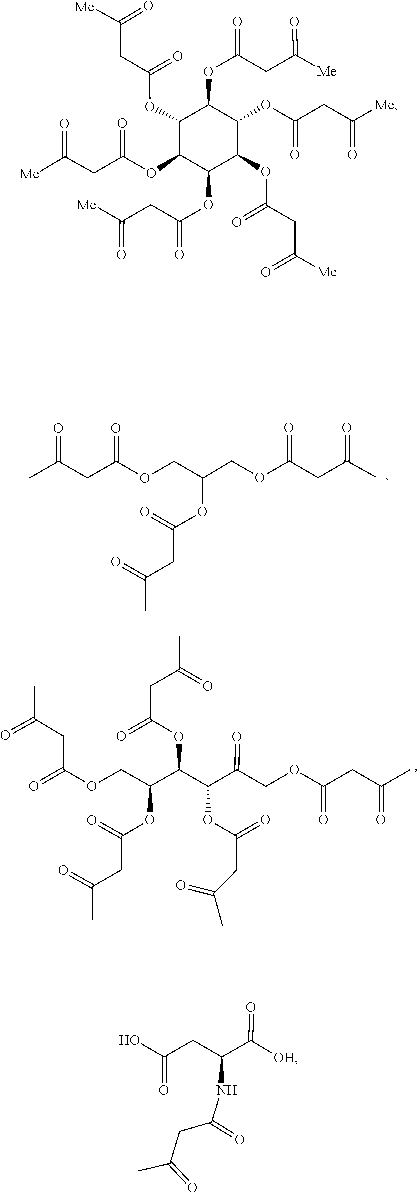

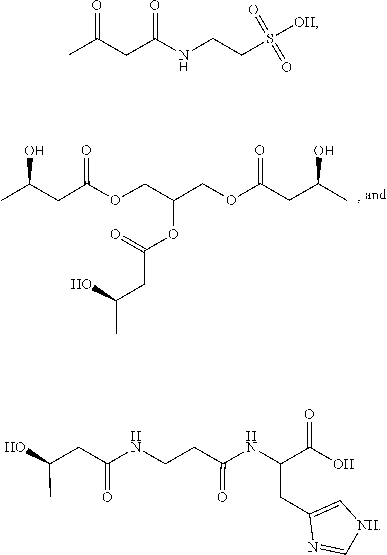 [SMILES: CC(=O)CC(=O)NCCS(=O)(=O)O ; C[C@H](O)CC(=O)OCC(COC(=O)C[C@@H](C)O)OC(=O)C[C@@H](C)O ; C[C@@H](O)CC(=O)NCCC(=O)NC(Cc1c[nH]cn1)C(=O)O]

In another aspect, provided herein is a method for treating non-alcoholic fatty liver disease (NAFLD), the method comprising administering to a subject a therapeutically effective amount of a compound of Formula (A):

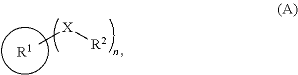

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

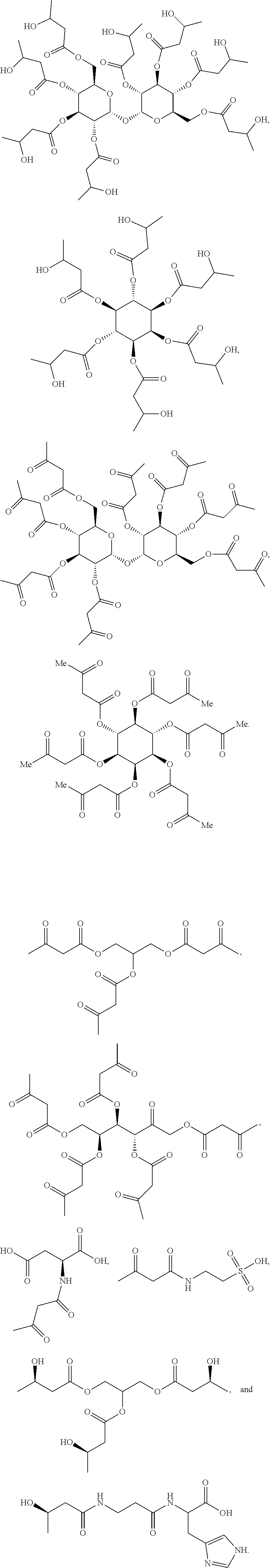

In another aspect, provided herein is a method for treating non-alcoholic steatohepatitis (NASH), the method comprising administering to a subject a therapeutically effective amount of a compound of Formula (A):

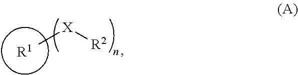

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

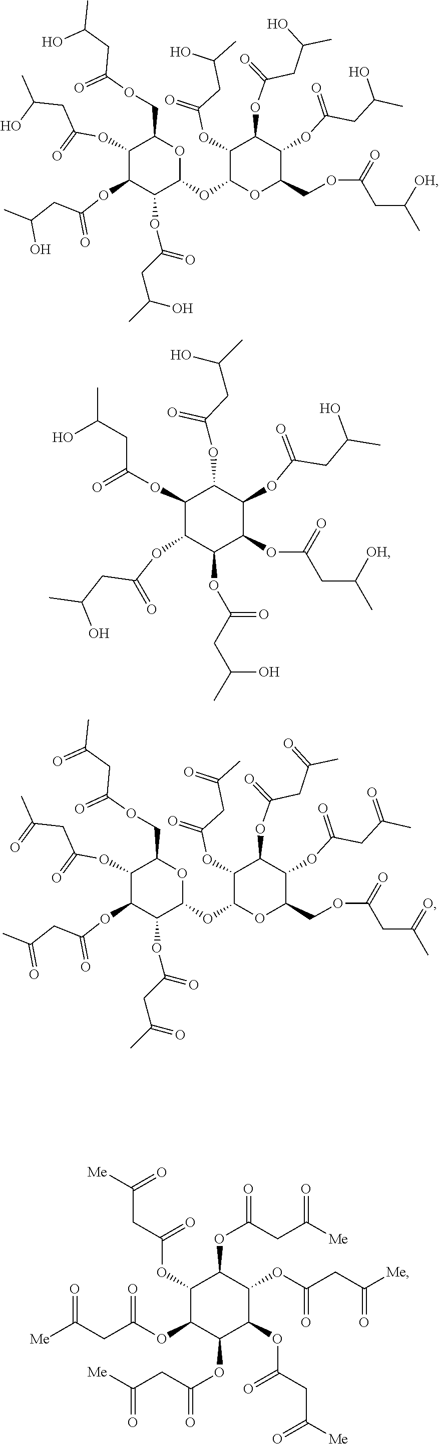

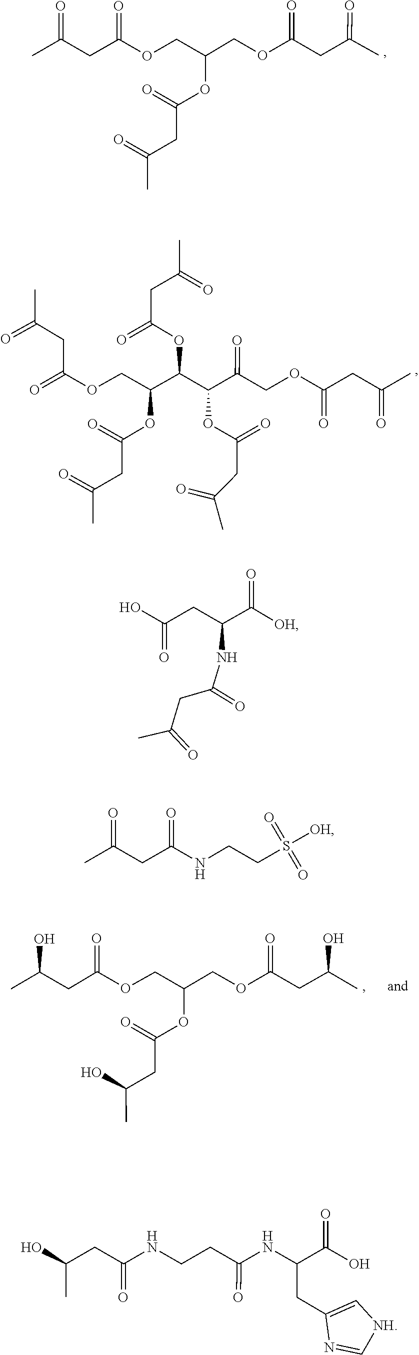

In another aspect, provided herein is a method for treating polycystic ovarian syndrome (PCOS), the method comprising administering to a subject a therapeutically effective amount of a compound of Formula (A):

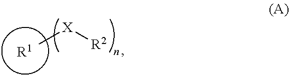

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

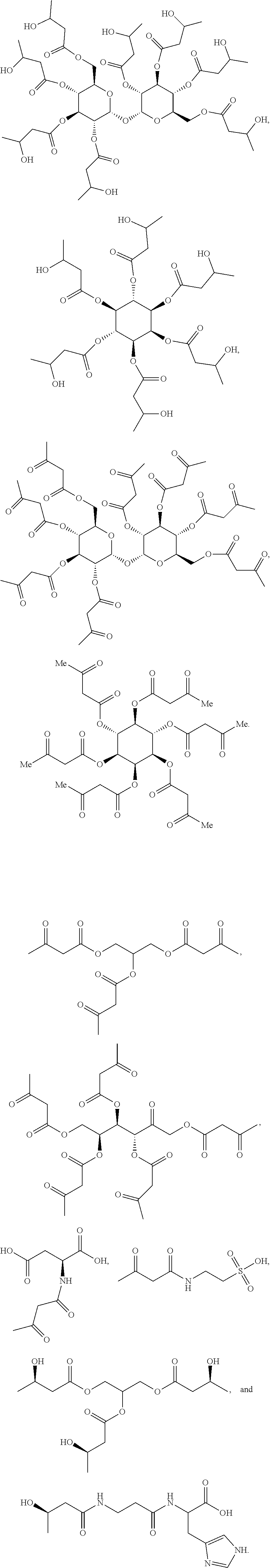

In another aspect, provided herein is a method for treating epilepsy, the method comprising administering to a subject a therapeutically effective amount of a compound of Formula (A):

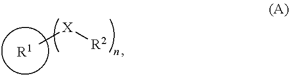

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

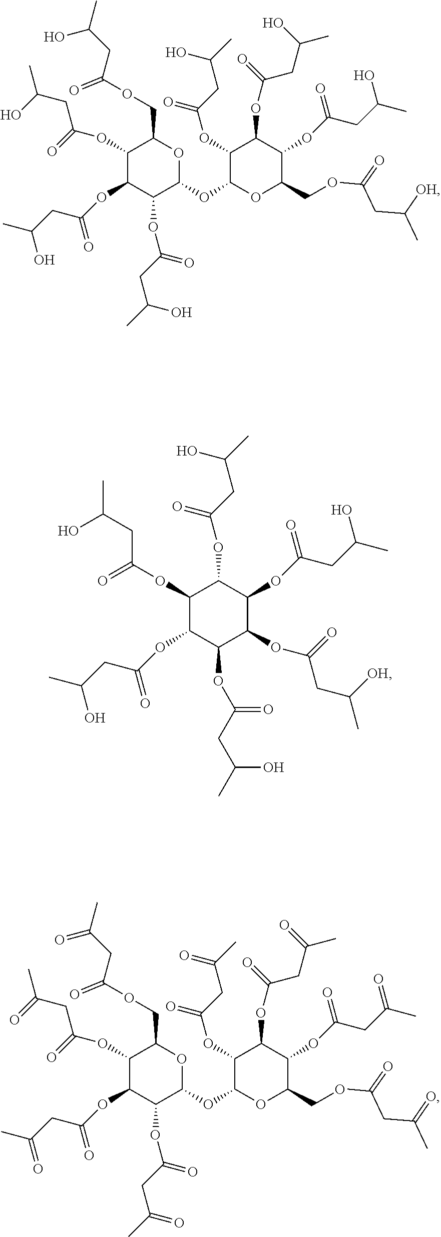

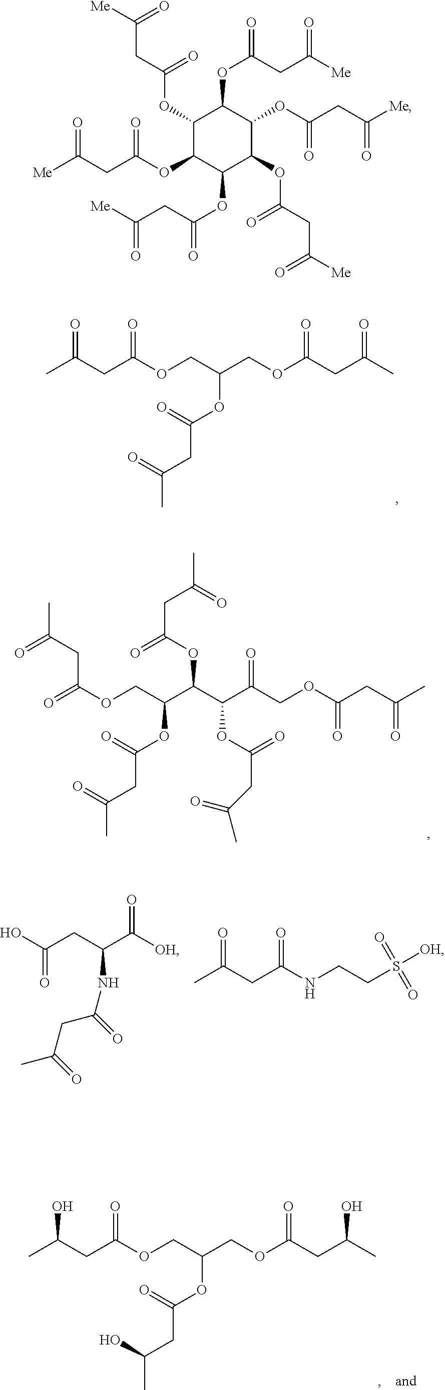

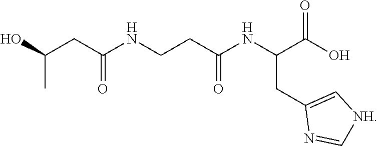 [SMILES: C[C@@H](O)CC(=O)NCCC(=O)NC(Cc1c[nH]cn1)C(=O)O]

In another aspect, provided herein is a method for treating traumatic brain injury (TBI), the method comprising administering to a subject a therapeutically effective amount of a compound of Formula (A):

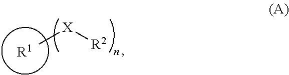

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

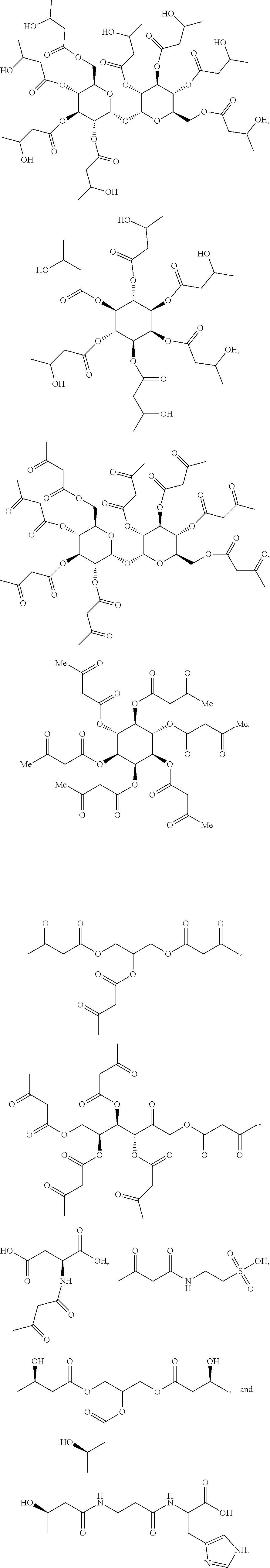

In another aspect, provided herein is a method for treating amyotrophic lateral sclerosis (ALS), the method comprising administering to a subject a therapeutically effective amount of a compound of Formula (A):

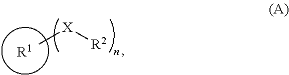

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

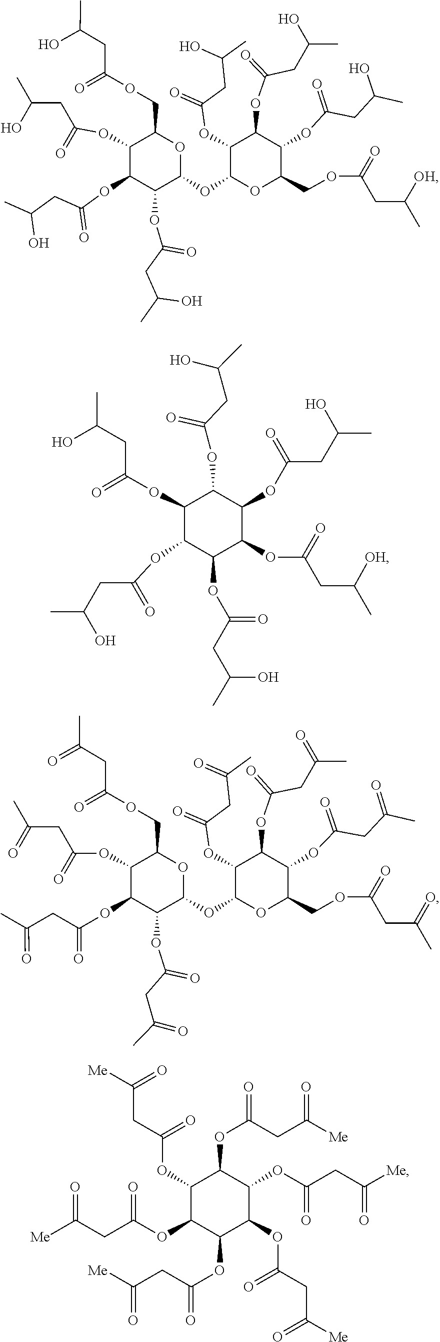

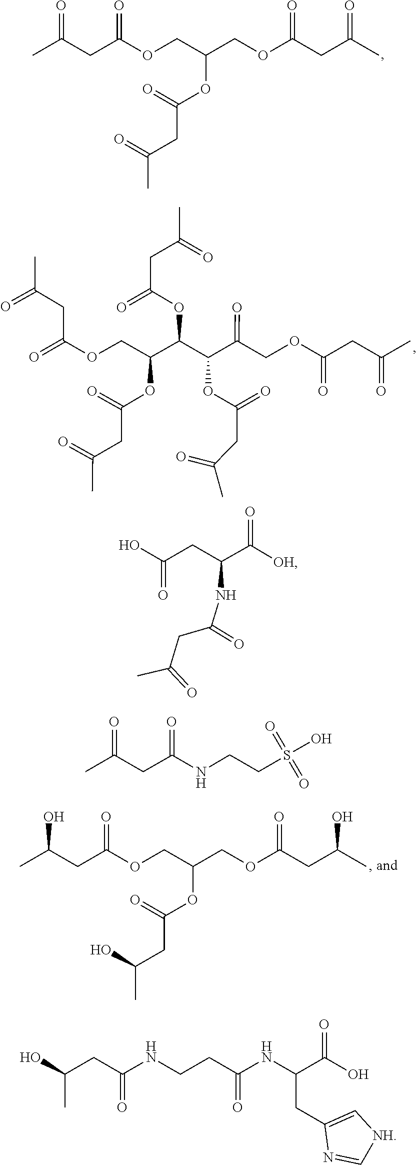

In another aspect, provided herein is a method for treating cancer, the method comprising administering to a subject a therapeutically effective amount of a compound of Formula (A):

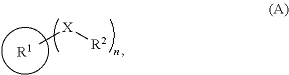

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

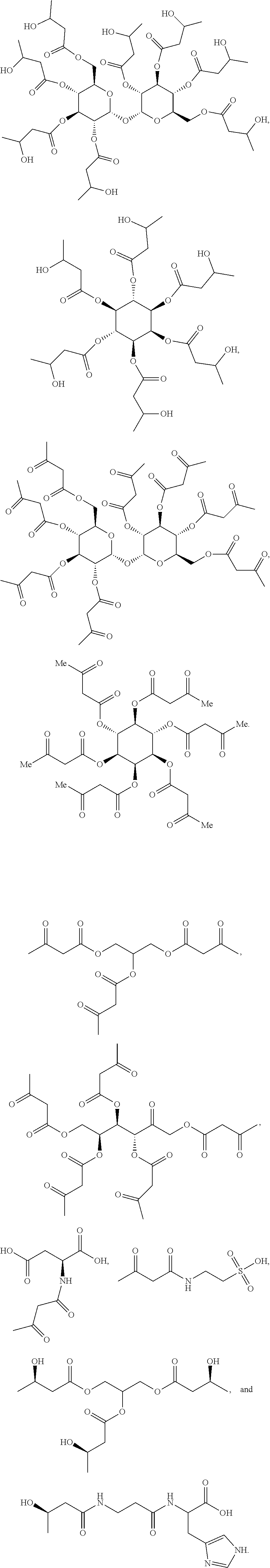

In another aspect, provided herein is a method for treating cancer cachexia, the method comprising administering to a subject a therapeutically effective amount of a compound of Formula (A):

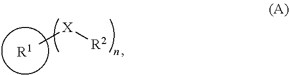

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

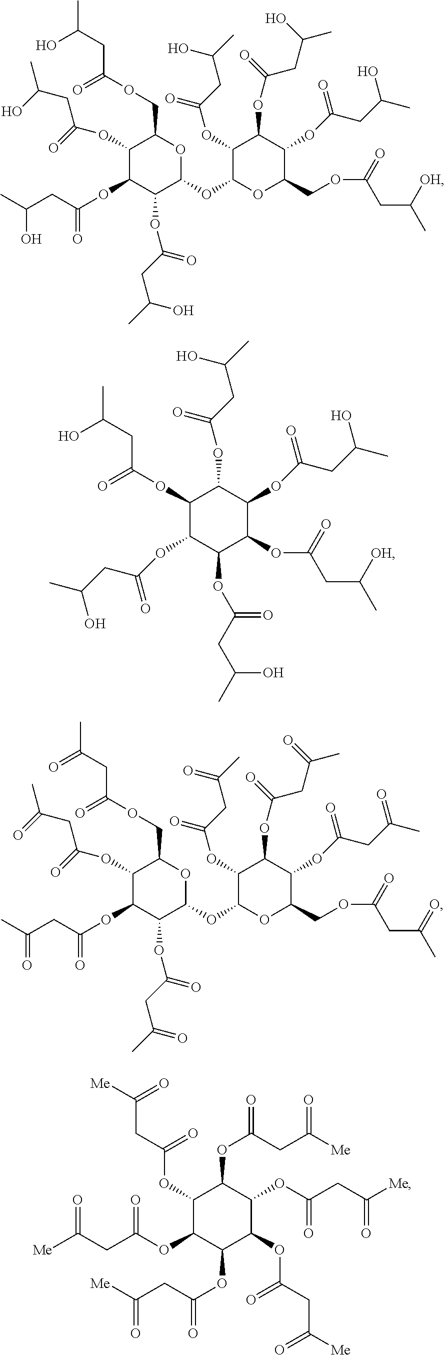

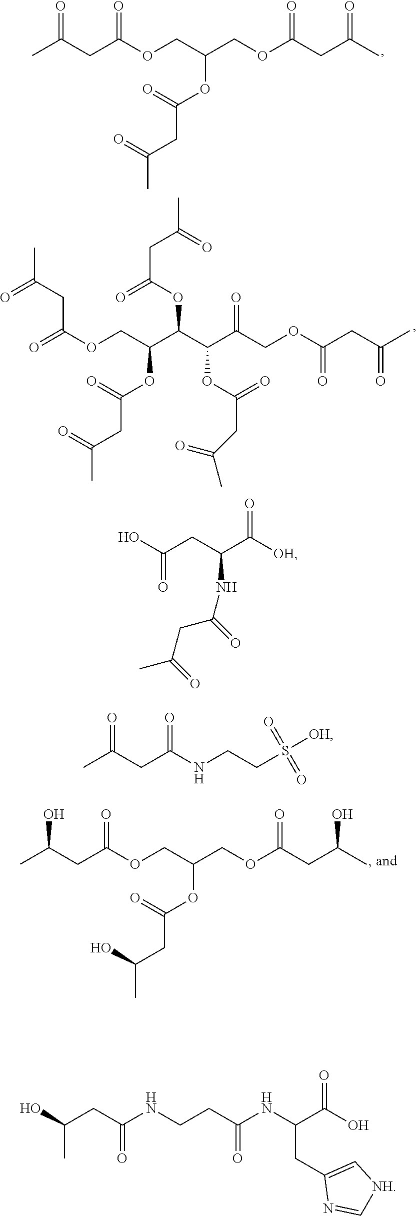

In another aspect, provided herein is a method for treating peripheral vascular disease, the method comprising administering to a subject a therapeutically effective amount of a compound of Formula (A):

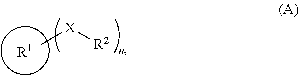

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

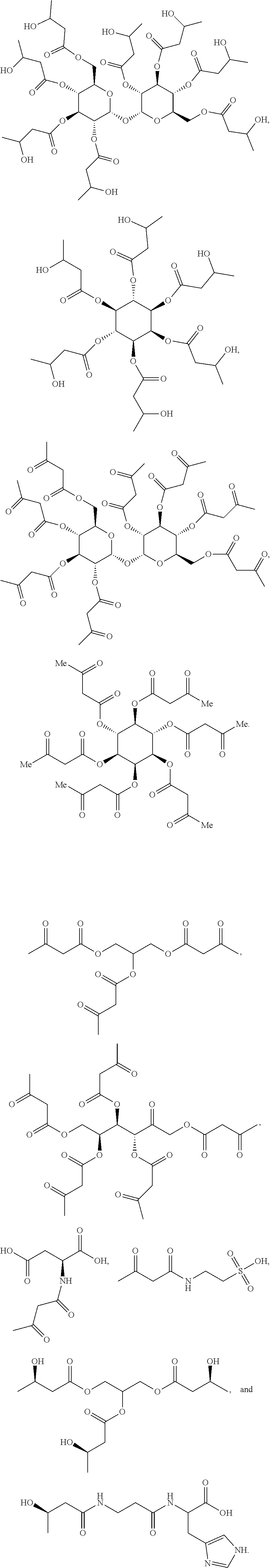

In another aspect, provided herein is a method for treating mitochondrial disorders, the method comprising administering to a subject a therapeutically effective amount of a compound of Formula (A):

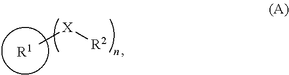

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

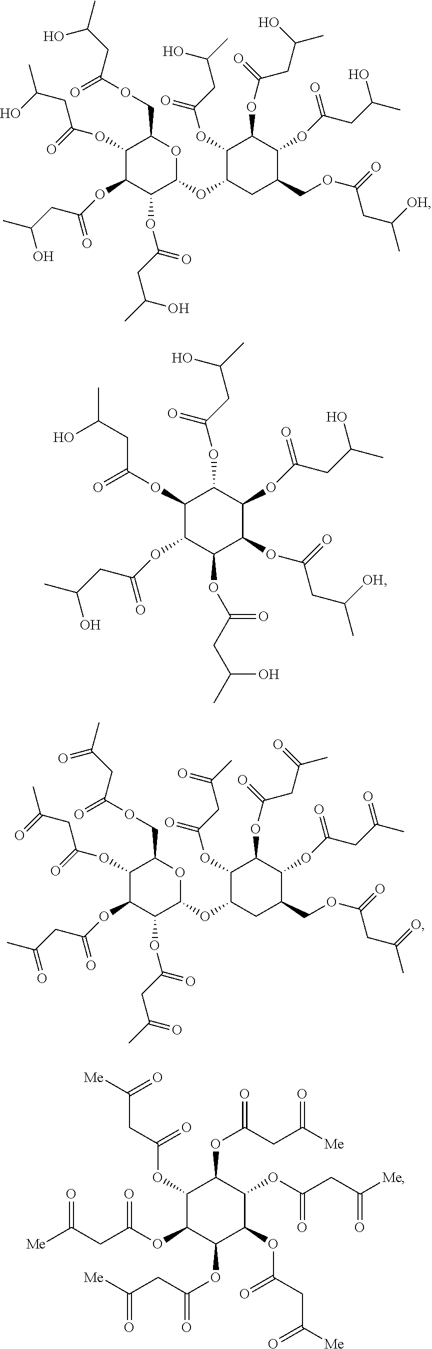

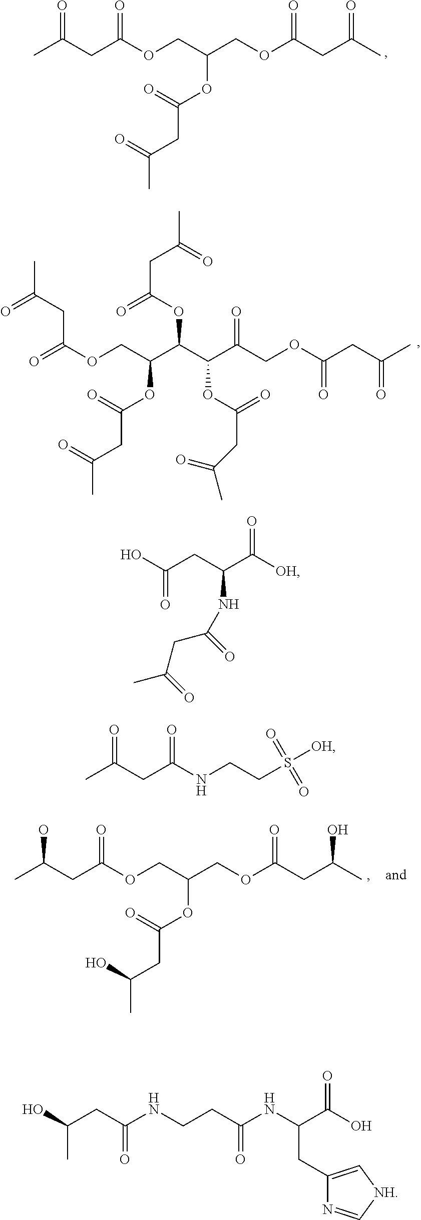

In another aspect, provided herein is a method for treating chronic fatigue syndrome, the method comprising administering to a subject a therapeutically effective amount of a compound of Formula (A):

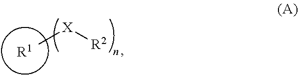

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

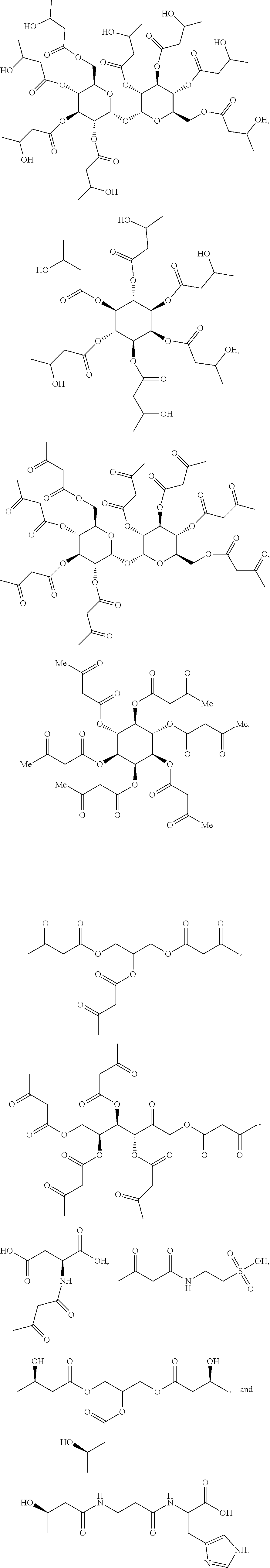

In another aspect, provided herein is a method for treating metabolic syndrome, the method comprising administering to a subject a therapeutically effective amount of a compound of Formula (A):

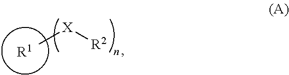

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

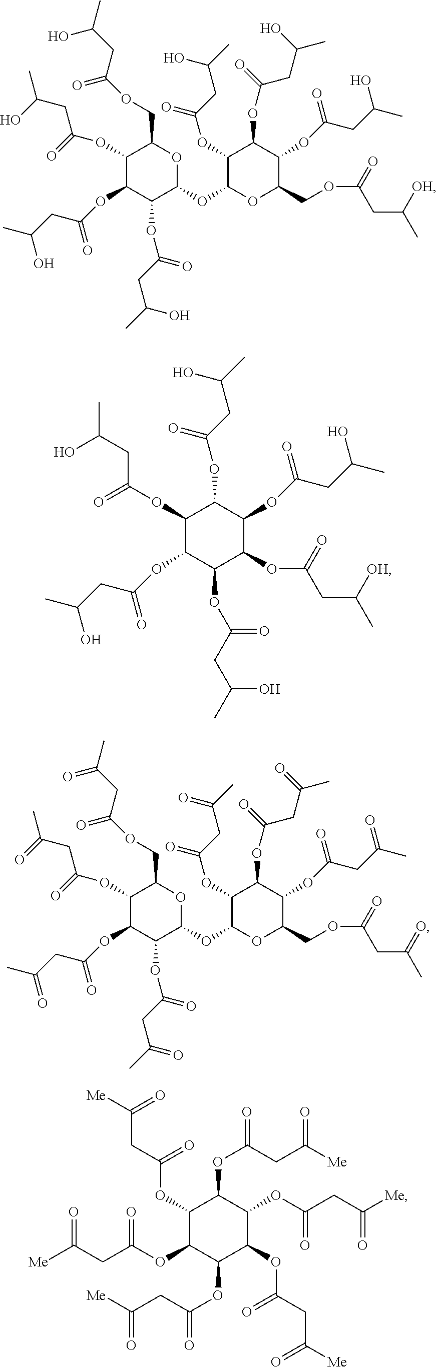

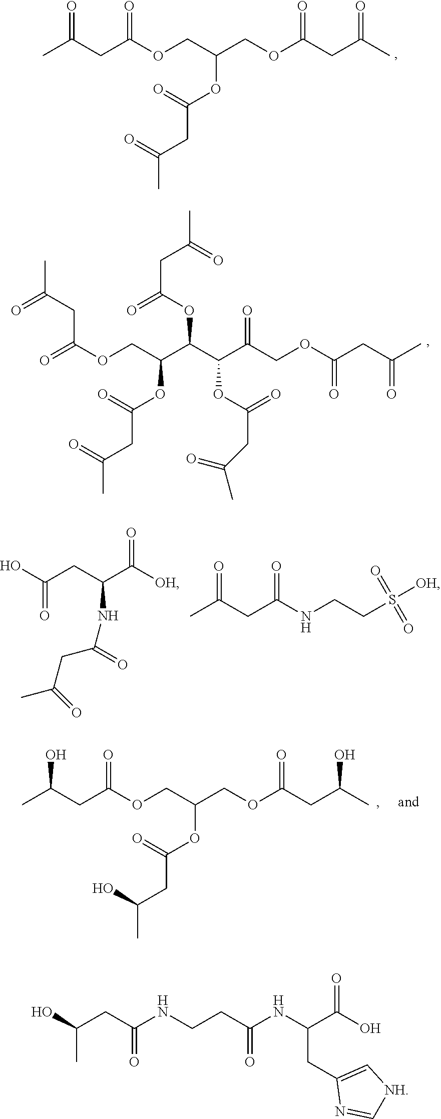

In another aspect, provided herein is a method for treating bipolar disorder, the method comprising administering to a subject a therapeutically effective amount of a compound of Formula (A):

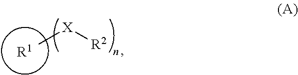

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

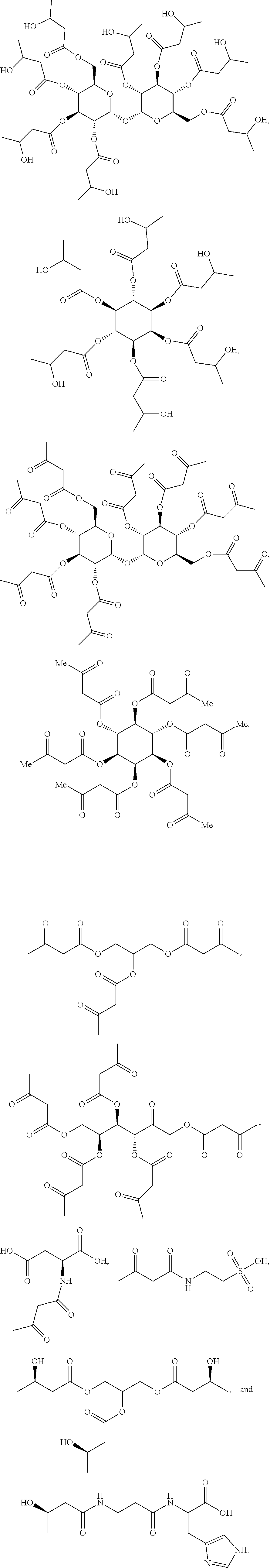

In another aspect, provided herein is a method for treating major depressive disorder, the method comprising administering to a subject a therapeutically effective amount of a compound of Formula (A):

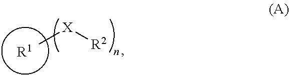

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

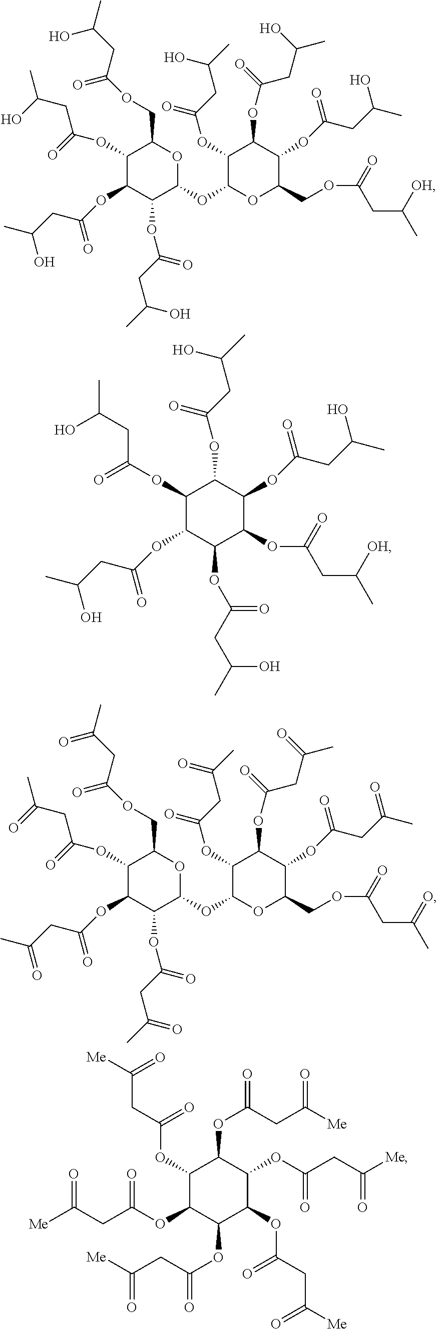

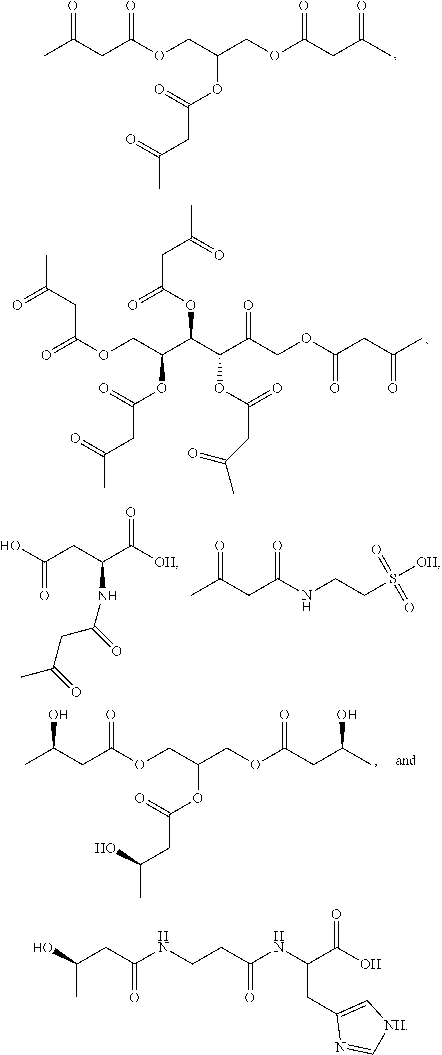

In another aspect, provided herein is a method for treating anxiety disorders, the method comprising administering to a subject a therapeutically effective amount of a compound of Formula (A):

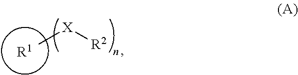

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

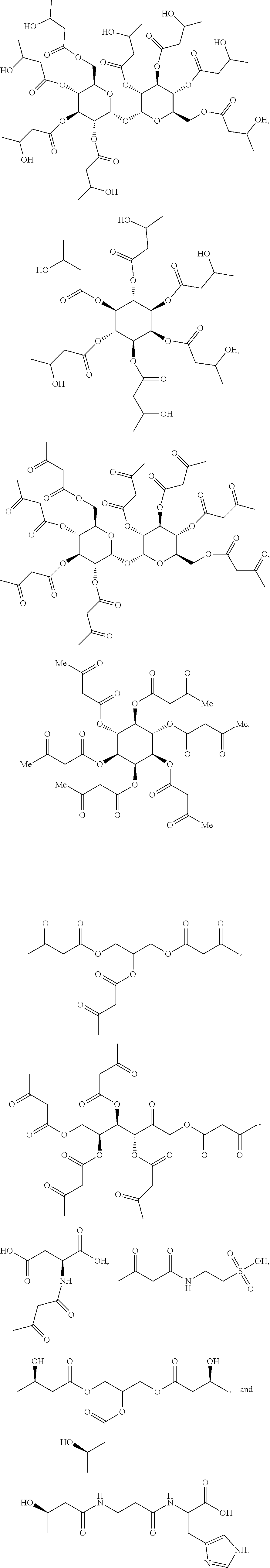

In another aspect, provided herein is a method for treating schizophrenia, the method comprising administering to a subject a therapeutically effective amount of a compound of Formula (A):

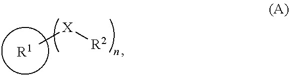

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

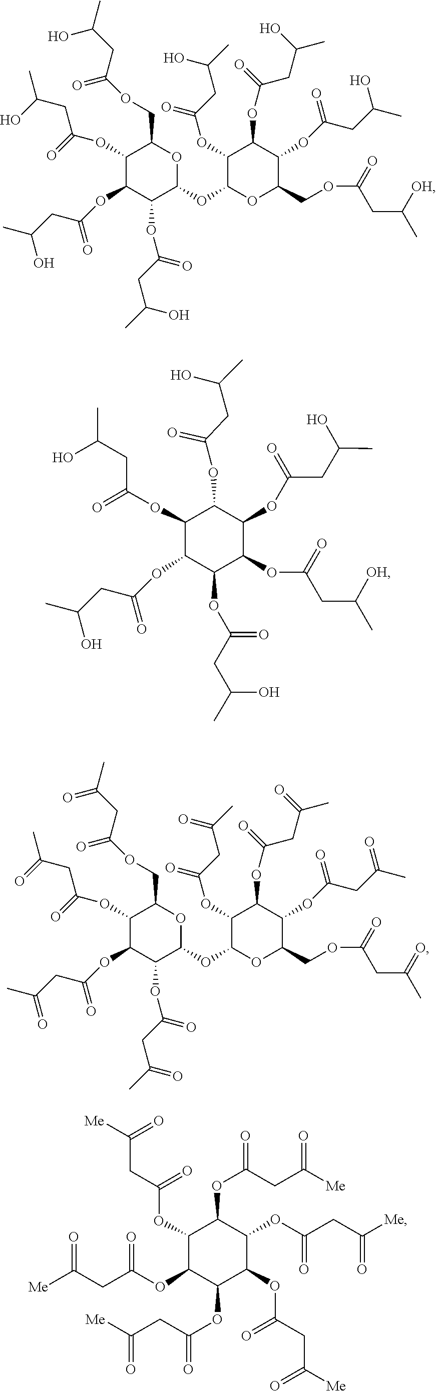

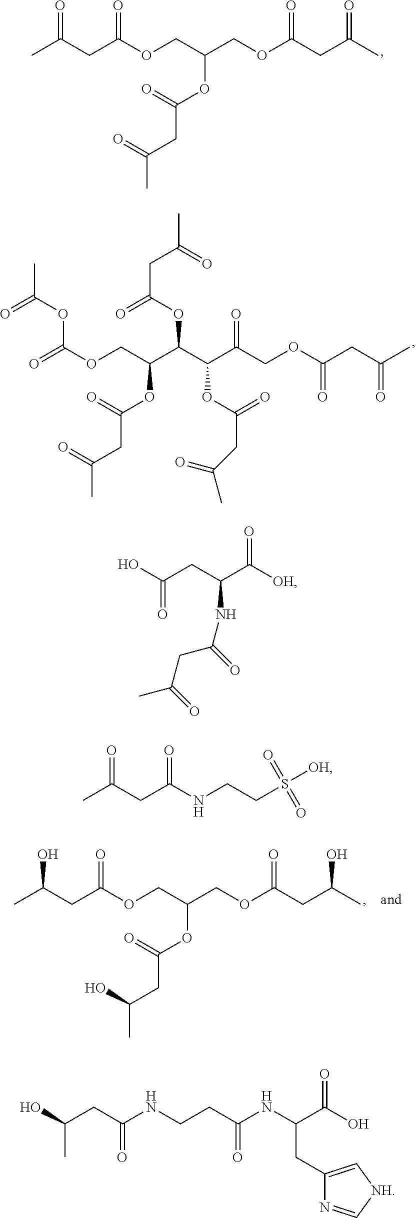

In another aspect, provided herein is a method for treating autism spectrum disorder, the method comprising administering to a subject a therapeutically effective amount of a compound of Formula (A):

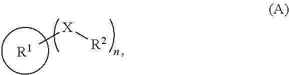

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

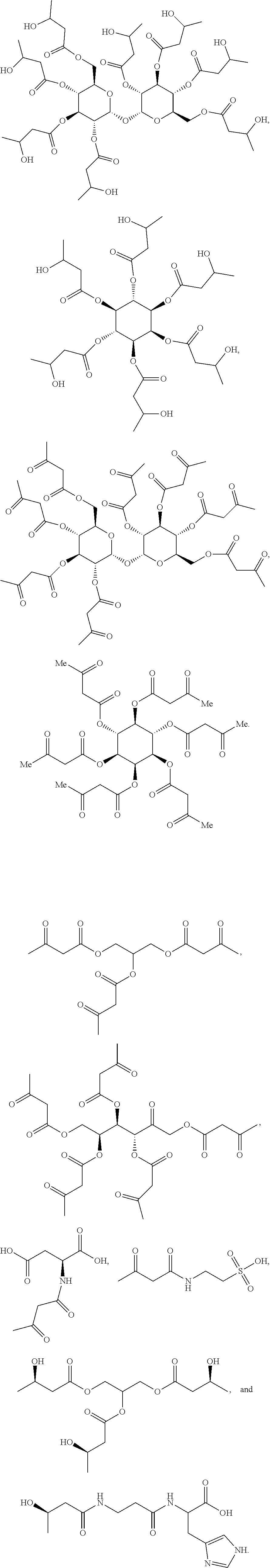

In another aspect, provided herein is a method for treating attention deficit hyperactivity disorder (ADHD), the method comprising administering to a subject a therapeutically effective amount of a compound of Formula (A)

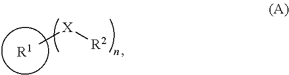

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

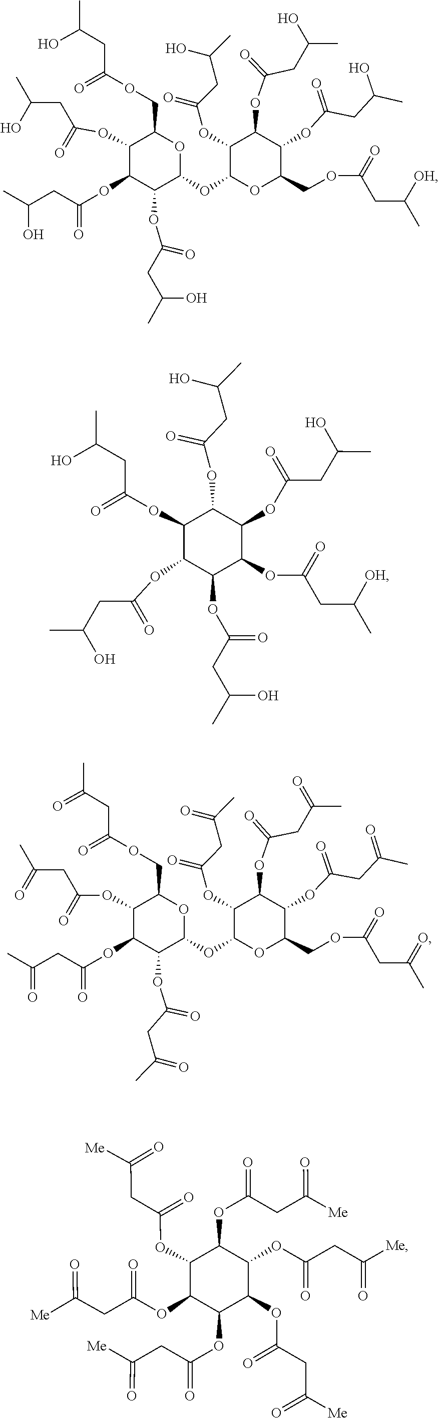

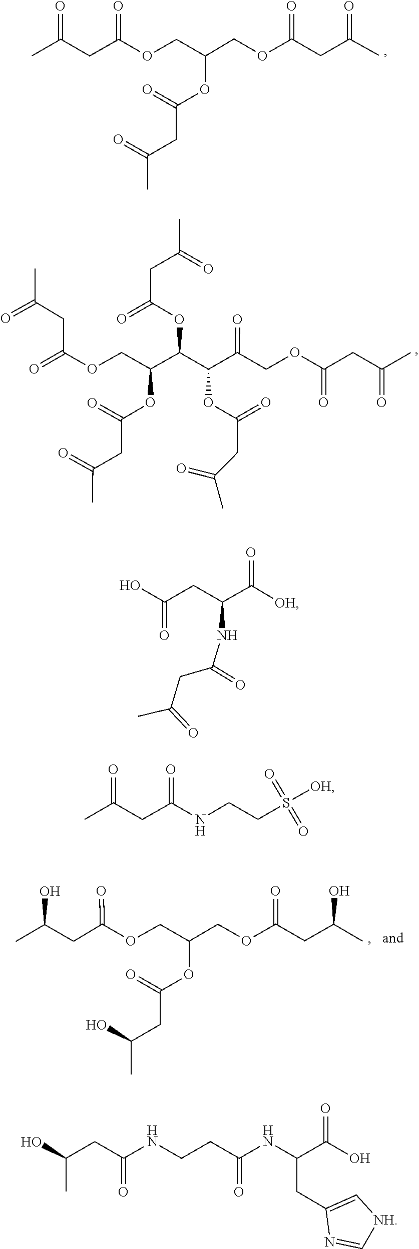

In another aspect, provided herein is a method for treating any condition in which a subject's tissue exhibits a quantifiable reduction in glucose utilization, the method comprising administering to a subject a therapeutically effective amount of a compound of Formula (A):

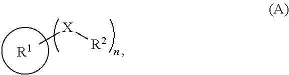

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In some embodiments, the compound of Formula (A) is a compound in Table 1. In some embodiments, the compound is selected from the group consisting of:

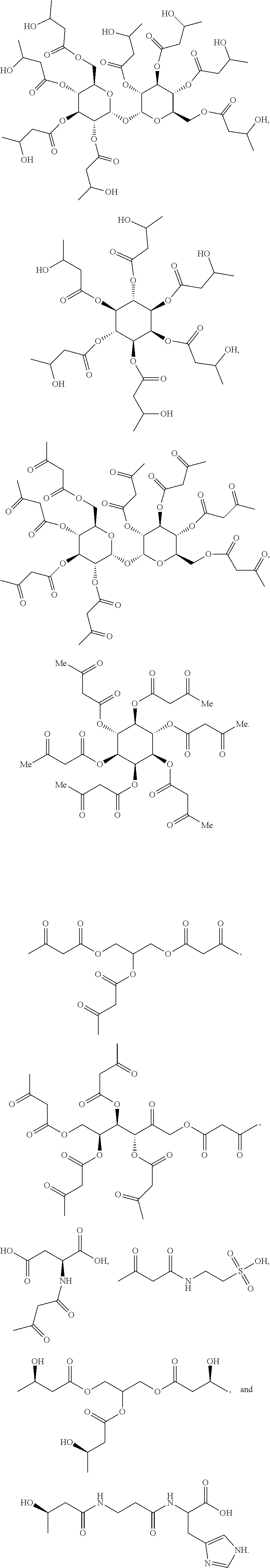

### Precise Measurement of Glucose Hypometabolism Across Conditions

Comprehensive, quantitative evidence demonstrates remarkably consistent patterns of glucose hypometabolism across diverse disorders:

### Alzheimer's Disease:

FDG-PET studies demonstrate 15-25% lower regional glucose metabolism in temporal, parietal, and posterior cingulate regions compared to age-matched controls. Early-onset AD shows even more dramatic declines, with 40-45% global cerebral metabolic rate glucose reduction. The degree of hypometabolism correlates strongly with cognitive impairment, with correlation coefficients between regional FDG uptake and cognitive scores of r≈0.5-0.7. Hypometabolism manifests 5-10 years before clinical dementia in sporadic AD and 13 years before expected symptom onset in familial AD.

### Parkinson's Disease:

Substantia nigra shows 15-20% lower FDG uptake in PD patients compared to controls (effect size d≈1.0). The PD-related metabolic pattern (PDRP) network expression correlates with motor UPDRS scores at r=0.62, p<0.005. Correlation between glucose metabolism deficits and dopaminergic neuron loss (DAT binding r~0.6) Heart Failure:

Cardiac glucose oxidation rates in failing hearts are 30-40% lower than in healthy hearts. Glucose's contribution to cardiac ATP production falls from approximately 30% in healthy hearts to 10-15% in severe heart failure. Myocardial phosphocreatine-to-ATP ratio (PCr/ATP) is reduced by 25-30% in dilated cardiomyopathy, correlating with LVEF at r~0.6

### Convergent Enzymatic and Mitochondrial Defects

A particularly surprising finding is the convergent pattern of enzymatic deficiencies across these diverse conditions:

TABLE 2
Comparison of Enzymatic Deficiencies in Alzheimer's
Disease, Parkinson' Disease, and Heart Failure

| | | Key | Magnitude | Energy |
|---|---|---|---|---|
| | Primary | Enzyme | of | Reserve |
| Disease | Tissue | Defect | Defect | Deficit |
| Alzheimer's | Brain | PDH | ~50% | ~20% ATP |
| Disease | cortex | complex | reduction | reduction |
| Parkinson's | Substantia | Complex I | ~30% | ~0-15% ATP |
| Disease | nigra | | reduction | reduction |
| Heart | Cardiac | PDH | 30-40% | ~30% ATP |
| Failure | tissue | complex | reduced | reduction |
| | | | flux | |

In one aspect, provided herein a method of treating a glucose hypometabolic disorder in a subject, the method comprising increasing glucose metabolism in one or more tissues of the subject.

In certain embodiments, the glucose hypometabolic disorder is selected from the group consisting of Alzheimer's disease, Parkinson's disease, Huntington's disease, heart failure, stroke, spinal cord injury, type 2 diabetes, obesity, non-alcoholic fatty liver disease (NAFLD), non-alcoholic steatohepatitis (NASH), polycystic ovary syndrome (PCOS), epilepsy, traumatic brain injury, amyotrophic lateral sclerosis, cancer, cancer cachexia, peripheral vascular disease, mitochondrial disorders, chronic fatigue syndrome, metabolic syndrome, bipolar disorder, major depressive disorder, anxiety disorders, schizophrenia, autism spectrum disorder, attention deficit hyperactivity disorder (ADHD), and any condition in which the subject's tissue exhibits a quantifiable reduction in glucose utilization.

In certain embodiments, the glucose hypometabolic disorder is Alzheimer's disease. In certain embodiments, the glucose hypometabolic disorder is Parkinson's disease. In certain embodiments, the glucose hypometabolic disorder is Huntington's disease. In certain embodiments, the glucose hypometabolic disorder is heart failure. In certain embodiments, the glucose hypometabolic disorder is stroke. In certain embodiments, the glucose hypometabolic disorder is spinal cord injury. In certain embodiments, the glucose hypometabolic disorder is type 2 diabetes. In certain embodiments, the glucose hypometabolic disorder is obesity. In certain embodiments, the glucose hypometabolic disorder is non-alcoholic fatty liver disease (NAFLD). In certain embodiments, the glucose hypometabolic disorder is non-alcoholic steatohepatitis (NASH). In certain embodiments, the glucose hypometabolic disorder is polycystic ovary syndrome (PCOS). In certain embodiments, the glucose hypometabolic disorder is epilepsy. In certain embodiments, the glucose hypometabolic disorder is traumatic brain injury. In certain embodiments, the glucose hypometabolic disorder is amyotrophic lateral sclerosis. In certain embodiments, the glucose hypometabolic disorder is cancer. In certain embodiments, the glucose hypometabolic disorder is cancer cachexia. In certain embodiments, the glucose hypometabolic disorder is peripheral vascular disease. In certain embodiments, the glucose hypometabolic disorder is a mitochondrial disorder. In certain embodiments, the glucose hypometabolic disorder is chronic fatigue syndrome. In certain embodiments, the glucose hypometabolic disorder is metabolic syndrome. In certain embodiments, the glucose hypometabolic disorder is bipolar disorder. In certain embodiments, the glucose hypometabolic disorder is major depressive disorder. In certain embodiments, the glucose hypometabolic disorder is an anxiety disorder. In certain embodiments, the glucose hypometabolic disorder is schizophrenia. In certain embodiments, the glucose hypometabolic disorder is autism spectrum disorder. In certain embodiments, the glucose hypometabolic disorder is attention deficit hyperactivity disorder (ADHD). In certain embodiments, the glucose hypometabolic disorder is any condition in which the subject's tissue exhibits a quantifiable reduction in glucose utilization.

In some embodiments, increasing glucose metabolism comprises increasing the activity of a glucose hypometabolic enzyme. In some embodiments, increasing glucose metabolism comprises increasing the activity of a pyruvate dehydrogenase complex (PDH complex).

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating Alzheimer's Disease in a subject, the method comprising increasing glucose metabolism in one or more tissues of the subject. In some embodiments, the one or more tissues comprises brain tissue. In some embodiments, increasing glucose metabolism comprises increasing the activity of a glucose hypometabolic enzyme. In some embodiments, increasing glucose metabolism comprises increasing the activity of a pyruvate dehydrogenase complex (PDH complex).

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%. In another aspect, provided herein is a method of treating Parkinson's Disease in a subject, the method comprising increasing glucose metabolism in one or more tissues of the subject. In some embodiments, the one or more tissues comprises brain tissue. In some embodiments, increasing glucose metabolism comprises increasing the activity of a glucose hypometabolic enzyme. In some embodiments, increasing glucose metabolism comprises increasing the activity of a pyruvate dehydrogenase complex (PDH complex).

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating Huntington's Disease in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating heart failure in a subject, the method comprising increasing glucose metabolism in one or more tissues of the subject. In some embodiments, the one or more tissues comprises heart tissue. In some embodiments, increasing glucose metabolism comprises increasing the activity of a glucose hypometabolic enzyme. In some embodiments, increasing glucose metabolism comprises increasing the activity of a pyruvate dehydrogenase complex (PDH complex).

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating stroke in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating spinal cord injury in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating type 2 diabetes in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating obesity in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating non-alcoholic fatty liver disease (NAFLD) in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating non-alcoholic steatohepatitis (NASH) in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating polycystic ovarian syndrome (PCOS) in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating epilepsy in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating traumatic brain injury (TBI) in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating amyotrophic lateral sclerosis (ALS) in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating cancer in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating cancer cachexia in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating peripheral vascular disease in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating mitochondrial disorders in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating chronic fatigue syndrome in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating metabolic syndrome in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating bipolar disorder in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating major depressive disorder in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating anxiety disorders in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating schizophrenia in a subject, the method comprising increasing glucose metabolism in one or more tissues of the subject. In some embodiments, the one or more tissues comprises brain tissue.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating autism spectrum disorder in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating attention deficit hyperactivity disorder (ADHD) in a subject, the method comprising increasing glucose metabolism in one or more tissues of the subject.

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In another aspect, provided herein is a method of treating any condition in which a subject's tissue exhibits a quantifiable reduction in glucose utilization, the method comprising increasing glucose metabolism in one or more tissues of the subject

In some embodiments, the glucose metabolism is increased by about 5% to 75%. In some embodiments, the glucose metabolism is increased by about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, or about 70 to 75%. In some embodiments, the glucose metabolism is increased by about 5% to 10%. In some embodiments, the glucose metabolism is increased by about 10% to 15%. In some embodiments, the glucose metabolism is increased by about 15% to 20%. In some embodiments, the glucose metabolism is increased by about 20% to 25%. In some embodiments, the glucose metabolism is increased by about 25% to 30%. In some embodiments, the glucose metabolism is increased by about 30% to 35%. In some embodiments, the glucose metabolism is increased by about 35% to 40%. In some embodiments, the glucose metabolism is increased by about 40% to 45%. In some embodiments, the glucose metabolism is increased by about 45% to 50%. In some embodiments, the glucose metabolism is increased by about 50% to 55%. In some embodiments, the glucose metabolism is increased by about 55% to 60%. In some embodiments, the glucose metabolism is increased by about 60% to 65%. In some embodiments, the glucose metabolism is increased by about 65% to 70%. In some embodiments, the glucose metabolism is increased by about 70 to 75%.

In some embodiments, the glucose metabolism is increased by about 5%, about 10%, about 15%, about 20%, about 25%, about 30%, about 35%, about 40%, about 45%, about 50%, about 55%, about 60%, about 65%, or about 70%, or about 75%. In some embodiments, the glucose metabolism is increased by about 5%. In some embodiments, the glucose metabolism is increased by about 10%. In some embodiments, the glucose metabolism is increased by about 15%. In some embodiments, the glucose metabolism is increased by about 20%. In some embodiments, the glucose metabolism is increased by about 25%. In some embodiments, the glucose metabolism is increased by about 30%. In some embodiments, the glucose metabolism is increased by about 35%. In some embodiments, the glucose metabolism is increased by about 40%. In some embodiments, the glucose metabolism is increased by about 45%. In some embodiments, the glucose metabolism is increased by about 50%. In some embodiments, the glucose metabolism is increased by about 55%. In some embodiments, the glucose metabolism is increased by about 60%. In some embodiments, the glucose metabolism is increased by about 65%. In some embodiments, the glucose metabolism is increased by about or about 70%. In some embodiments, the glucose metabolism is increased by about 75%.

In some embodiments, increasing glucose metabolism comprises increasing the production of adenosine triphosphate (ATP). In some embodiments, the production of ATP is increased by about 5% to 50%. In some embodiments, the production of ATP is increased by about 5% to 10%, about 10% to 15%, about 15% to 20%, about 20% to 25%, about 25% to 30%, about 30% to 35%, about 35% to 40%, about 40% to 45%, about 45% to 50%. In some embodiments, the production of ATP is increased by about 5% to 10%. In some embodiments, the production of ATP is increased by about 10% to 15%. In some embodiments, the production of ATP is increased by about 15% to 20%. In some embodiments, the production of ATP is increased by about 20% to 25%. In some embodiments, the production of ATP is increased by about 25% to 30%. In some embodiments, the production of ATP is increased by about 30% to 35%. In some embodiments, the production of ATP is increased by about 35% to 40%. In some embodiments, the production of ATP is increased by about 40% to 45%. In some embodiments, the production of ATP is increased by about 45% to 50%.

In some embodiments, the production of ATP is increased by about 5%, about 10%, about 15%, about 20%, about 25%, about 30%, about 35%, about 40%, about 45%, about 50%. In some embodiments, the production of ATP is increased by about 5%. In some embodiments, the production of ATP is increased by about 10%. In some embodiments, the production of ATP is increased by about 15%. In some embodiments, the production of ATP is increased by about 20%. In some embodiments, the production of ATP is increased by about 25%. In some embodiments, the production of ATP is increased by about 30%. In some embodiments, the production of ATP is increased by about 35%. In some embodiments, the production of ATP is increased by about 40%. In some embodiments, the production of ATP is increased by about 45%. In some embodiments, the production of ATP is increased by about 50%.

In certain embodiments, the method disclosed herein results in an increase in ATP production, restoration of cellular energy metabolism, compensation for glucose hypometabolism, improvement in metabolic efficiency, improvement in mitochondrial respiration, improvement in basal respiratory capacity, improvement in mitochondrial function, increase in oxygen consumption rate (OCR), improvement in neuronal viability, reduction in neuronal cell death, reduction in apoptosis, protection against amyloid-induced toxicity, promotion of neuronal survival, reduction in reactive oxygen species (ROS), reduction in oxidative stress, improvement in mitochondrial integrity, increased microglial uptake of amyloid beta, increased clearance of amyloid beta, acceleration of amyloid clearance kinetics, improvement in cognitive function, stabilization of cognitive decline, improvement in memory, improvement in alertness, improvement in language function, improvement in overall cognitive performance, improvement in cardiac output, improvement in cardiac function, improvement in ejection fraction, reduction in blood glucose levels, improvement in glycemic control, reduction in HbA1c, improvement in insulin sensitivity, reduction in insulin requirements, reduction in body weight, reduction in fat mass, reduction in liver fat, improvement in liver function, reduction in hepatic inflammation, increased time-in-range for glucose control, stabilization of metabolic parameters, increased energy levels, reduced fatigue, reduced sugar cravings, improved quality of life, sustained elevation of blood ketone levels, improved tolerability, and controlled or prolonged ketone release.

In certain embodiments, the method disclosed herein results in an increase in ATP production. In certain embodiments, the method disclosed herein results in restoration of cellular energy metabolism. In certain embodiments, the method disclosed herein results in compensation for glucose hypometabolism. In certain embodiments, the method disclosed herein results in improvement in metabolic efficiency. In certain embodiments, the method disclosed herein results in improvement in mitochondrial respiration. In certain embodiments, the method disclosed herein results in improvement in basal respiratory capacity. In certain embodiments, the method disclosed herein results in improvement in mitochondrial function. In certain embodiments, the method disclosed herein results in an increase in oxygen consumption rate (OCR). In certain embodiments, the method disclosed herein results in improvement in neuronal viability. In certain embodiments, the method disclosed herein results in reduction in neuronal cell death. In certain embodiments, the method disclosed herein results in reduction in apoptosis. In certain embodiments, the method disclosed herein results in protection against amyloid-induced toxicity. In certain embodiments, the method disclosed herein results in promotion of neuronal survival. In certain embodiments, the method disclosed herein results in reduction in reactive oxygen species (ROS). In certain embodiments, the method disclosed herein results in reduction in oxidative stress. In certain embodiments, the method disclosed herein results in improvement in mitochondrial integrity. In certain embodiments, the method disclosed herein results in increased microglial uptake of amyloid beta. In certain embodiments, the method disclosed herein results in increased clearance of amyloid beta. In certain embodiments, the method disclosed herein results in acceleration of amyloid clearance kinetics. In certain embodiments, the method disclosed herein results in improvement in cognitive function. In certain embodiments, the method disclosed herein results in stabilization of cognitive decline. In certain embodiments, the method disclosed herein results in improvement in memory. In certain embodiments, the method disclosed herein results in improvement in alertness. In certain embodiments, the method disclosed herein results in improvement in language function. In certain embodiments, the method disclosed herein results in improvement in overall cognitive performance. In certain embodiments, the method disclosed herein results in improvement in cardiac output. In certain embodiments, the method disclosed herein results in improvement in cardiac function. In certain embodiments, the method disclosed herein results in improvement in ejection fraction. In certain embodiments, the method disclosed herein results in reduction in blood glucose levels. In certain embodiments, the method disclosed herein results in improvement in glycemic control. In certain embodiments, the method disclosed herein results in reduction in HbA1c. In certain embodiments, the method disclosed herein results in improvement in insulin sensitivity. In certain embodiments, the method disclosed herein results in reduction in insulin requirements. In certain embodiments, the method disclosed herein results in reduction in body weight. In certain embodiments, the method disclosed herein results in reduction in fat mass. In certain embodiments, the method disclosed herein results in reduction in liver fat. In certain embodiments, the method disclosed herein results in improvement in liver function. In certain embodiments, the method disclosed herein results in reduction in hepatic inflammation. In certain embodiments, the method disclosed herein results in increased time-in-range for glucose control. In certain embodiments, the method disclosed herein results in stabilization of metabolic parameters. In certain embodiments, the method disclosed herein results in increased energy levels. In certain embodiments, the method disclosed herein results in reduced fatigue. In certain embodiments, the method disclosed herein results in reduced sugar cravings. In certain embodiments, the method disclosed herein results in improved quality of life. In certain embodiments, the method disclosed herein results in sustained elevation of blood ketone levels. In certain embodiments, the method disclosed herein results in improved tolerability. In certain embodiments, the method disclosed herein results in controlled or prolonged ketone release.

This previously unrecognized pattern of convergent enzymatic deficiencies at remarkably similar magnitudes (30-50% reductions) strongly supports a common metabolic pathophysiology and points to potential common therapeutic targets.

### Preserved Ketone Metabolism: The Critical Therapeutic Opportunity

The most significant and therapeutically relevant finding is that ketone metabolism remains intact or is enhanced across these conditions despite impaired glucose utilization:

### Alzheimer's Disease

Three independent PET studies using radiolabeled ketones confirmed that AD brains readily oxidize ketones at normal rates despite significant glucose hypometabolism.

### Parkinson's Disease

PD brain ketone utilization appears preserved, with preclinical models showing ketones effectively utilized and providing neuroprotection.

### Heart Failure

Failing hearts demonstrate ~2× higher ketone uptake and utilization than normal hearts, with upregulation of ketone-specific enzymes (BDH1, SCOT) and transporters.

This differential preservation of ketone metabolism despite glucose hypometabolism represents a critical therapeutic opportunity that forms the core of the present invention.

### Therapeutic Efficacy Across Diverse Conditions

Robust clinical evidence demonstrates that addressing the common glucose hypometabolism through ketone therapy produces significant and consistent benefits across these diverse conditions:

### Controlled Clinical Evidence of Efficacy

### Alzheimer's Disease:

In one randomized controlled clinical study (n=152), APOE-ε4-negative patients on ketogenic a compound of Formula (A)C-1202 improved by +4.7 points versus placebo on the ADAS-Cog at Day 45 (p=0.0005) and by +3.4 points at Day 90 (p=0.0148). Even in the overall intention-to-treat sample, a 1.9-point ADAS-Cog improvement (p=0.02) was observed. 5 of 7 clinical studies of ketogenic interventions in MCI/AD showed significant cognitive or metabolic improvements.

### Parkinson's Disease:

In one randomized controlled clinical study (n=47), 8-week ketogenic diet produced 41% improvement in non-motor symptoms (MDS-UPDRS Part I) versus 11% with low-fat diet (p<0.001). Therapeutic ketosis led to improved p-hydroxybutyrate levels of ~2-3 mM, associated with symptom improvements.

### Heart Failure:

In clinical settings, administration of intravenous β-hydroxybutyrate to achieve blood concentrations of approximately 3 mM increased cardiac output by 2.0 L/min (~30% increase) without increasing myocardial oxygen consumption. SGLT2 inhibitors (which induce mild ketosis) demonstrated ~25% reductions in HF hospitalization risk. Animal models: 4-week ketone ester improved ejection fraction by ~15 percentage points.

### Dose-Response Relationship Across Conditions

A key finding supporting the unified mechanism is a consistent dose-response relationship between circulating ketone levels and clinical improvement across multiple disease states. For example, higher serum ketone levels are associated with greater cognitive improvement in subjects with Alzheimer's disease. Similarly, in Parkinson's disease, ketone levels in the range of approximately 2-3 mM are associated with greater improvement in clinical symptoms. In heart failure, blood p-hydroxybutyrate levels of approximately 3 mM are associated with increases in cardiac output on the order of approximately 30%. This consistent relationship between ketone levels and clinical improvement across diverse conditions strongly supports the mechanistic validity of the disclosed approach.

### Mechanistic Details

### Metabolic Bypass Mechanism

The invention leverages the critical insight that ketone bodies (p-hydroxybutyrate, acetoacetate) can bypass the glucose-specific metabolic blockade observed in these conditions:

In Alzheimer's and heart failure, pyruvate dehydrogenase (PDH) complex activity is reduced by 30-50%, creating a bottleneck for glucose-derived pyruvate to enter the TCA cycle. Ketone bodies are metabolized to acetyl-CoA via a distinct enzymatic pathway (β-hydroxybutyrate dehydrogenase→acetoacetate→acetoacetyl-CoA→acetyl-CoA) that entirely bypasses the PDH complex. In Parkinson's disease, Complex I deficiency (~30% reduced) limits NADH oxidation from glucose metabolism; ketone metabolism generates FADH2 that can enter the electron transport chain at Complex II, bypassing the Complex I deficiency. This metabolic bypass mechanism explains why ketone therapy is effective across these seemingly disparate conditions.

### Energy Yield and Efficiency Advantages

The invention further leverages previously unappreciated energetic advantages of ketone metabolism. Ketone bodies produce more ATP per oxygen molecule consumed compared to glucose (2.5 vs 2.25 ATP/O), providing energetic efficiency in oxygen-limited conditions. Ketone metabolism produces fewer reactive oxygen species than glucose or fatty acid oxidation, reducing oxidative stress that is common across these conditions. Ketones have direct signaling effects that activate mitochondrial biogenesis and antioxidant pathways (e.g., Nrf2), providing benefits beyond simple energy provision.

### Methods of Treatment

One aspect of the disclosure provides a method of treating a subject diagnosed with a glucose hypometabolic disorder, comprising:
(a) measuring a glucose metabolism deficit (G_Diff) in one or more tissues of the subject using at least one diagnostic technique to obtain a quantitative indication of the subject's impaired glucose utilization;
(b) calculating a personalized exogenous ketone dosage (K) based on the measured G_Diff, wherein said calculation comprises applying at least one algorithm that correlates the measured G_Diff to the exogenous ketone intake required to at least partially compensate for the subject's impaired glucose utilization; and
(c) administering an exogenous ketone composition to the subject in accordance with the calculated personalized exogenous ketone dosage,
wherein the administered exogenous ketone composition provides an alternative metabolic fuel to the affected tissues, thereby addressing the subject's glucose hypometabolism.

In certain embodiments, the at least one diagnostic technique is selected from positron emission tomography (PET), magnetic resonance spectroscopy (MRS), metabolic assays, and wearable biosensors.

In certain embodiments, the calculation in step (b) comprises computing the personalized exogenous ketone dosage (K) using Formula (I):

$$K = G\_Diff \times ( 4 / 4.7 ) \times F \times p \qquad ( I )$$

wherein,
G_Diff is the measured glucose deficit in grams or equivalent energy units;
(4/4.7) is a factor accounting for relative energy yields of glucose versus ketones;
F is an efficiency factor (F≥1) reflecting systemic overhead or disease-specific factors; and p is a partition factor (0<p≤1) representing tissue-specific ketone uptake capacity.

It will be appreciated that Formula (I) is one exemplary approach to determining the ketone dosage K based on G_Diff. In other embodiments, the personalized ketone dosage can be calculated using different algorithms or computational techniques. For instance, a machine-learning model (trained on data from numerous patients) may predict an optimal ketone dose by taking the measured G_Diff as well as additional patient-specific parameters into account (such as the patient's age, liver function, current diet, or oxygen saturation). In another embodiment, a simple lookup table or an iterative titration protocol is used: the system might start at a baseline ketone dose and adjust upward or downward in controlled increments until the patient's metabolic markers reach target ranges. The computational module performing the dose calculation can be implemented locally (e.g., on a wearable device or pump controller) or remotely (e.g., a cloud server that receives the diagnostic data and returns a dosing recommendation). In all cases, the core principle is that the dose is quantitatively based on the measured glucose deficit, regardless of the specific mathematical model used. This ensures that even if different formulae or algorithms are employed, they fall within the scope of using G_Diff to inform therapy.

In certain embodiments, the glucose hypometabolic disorder is selected from the group consisting of Alzheimer's disease, Parkinson's disease, Huntington's disease, heart failure, stroke, spinal cord injury, type 2 diabetes, obesity, non-alcoholic fatty liver disease (NAFLD), non-alcoholic steatohepatitis (NASH), polycystic ovary syndrome (PCOS), epilepsy, traumatic brain injury, amyotrophic lateral sclerosis, cancer, cancer cachexia, peripheral vascular disease, mitochondrial disorders, chronic fatigue syndrome, metabolic syndrome, bipolar disorder, major depressive disorder, anxiety disorders, schizophrenia, autism spectrum disorder, attention deficit hyperactivity disorder (ADHD), and any condition in which the subject's tissue exhibits a quantifiable reduction in glucose utilization.

In certain embodiments, the glucose hypometabolic disorder is Alzheimer's disease. In certain embodiments, the glucose hypometabolic disorder is Parkinson's disease. In certain embodiments, the glucose hypometabolic disorder is Huntington's disease. In certain embodiments, the glucose hypometabolic disorder is heart failure. In certain embodiments, the glucose hypometabolic disorder is stroke. In certain embodiments, the glucose hypometabolic disorder is spinal cord injury. In certain embodiments, the glucose hypometabolic disorder is type 2 diabetes. In certain embodiments, the glucose hypometabolic disorder is obesity. In certain embodiments, the glucose hypometabolic disorder is non-alcoholic fatty liver disease (NAFLD). In certain embodiments, the glucose hypometabolic disorder is non-alcoholic steatohepatitis (NASH). In certain embodiments, the glucose hypometabolic disorder is polycystic ovary syndrome (PCOS). In certain embodiments, the glucose hypometabolic disorder is epilepsy. In certain embodiments, the glucose hypometabolic disorder is traumatic brain injury. In certain embodiments, the glucose hypometabolic disorder is amyotrophic lateral sclerosis. In certain embodiments, the glucose hypometabolic disorder is cancer. In certain embodiments, the glucose hypometabolic disorder is cancer cachexia. In certain embodiments, the glucose hypometabolic disorder is peripheral vascular disease. In certain embodiments, the glucose hypometabolic disorder is a mitochondrial disorder. In certain embodiments, the glucose hypometabolic disorder is chronic fatigue syndrome. In certain embodiments, the glucose hypometabolic disorder is metabolic syndrome. In certain embodiments, the glucose hypometabolic disorder is bipolar disorder. In certain embodiments, the glucose hypometabolic disorder is major depressive disorder. In certain embodiments, the glucose hypometabolic disorder is an anxiety disorder. In certain embodiments, the glucose hypometabolic disorder is schizophrenia. In certain embodiments, the glucose hypometabolic disorder is autism spectrum disorder. In certain embodiments, the glucose hypometabolic disorder is attention deficit hyperactivity disorder (ADHD). In certain embodiments, the glucose hypometabolic disorder is any condition in which the subject's tissue exhibits a quantifiable reduction in glucose utilization.

In certain embodiments, the method further comprises classifying the subject's disease severity into one or more metabolic stages (Stage 0 through 4) based on the measured G_Diff, and increasing the exogenous ketone dosage for each advancing metabolic stage to at least partially offset the subject's progressively reduced glucose utilization.

In certain embodiments, measuring the glucose metabolism deficit (G_Diff) comprises performing a fluorodeoxyglucose PET (FDG-PET) scan to quantify local or global glucose uptake in the subject's tissue, and comparing the measured local or global glucose uptake to a reference level to determine a magnitude of G_Diff, wherein the calculated personalized exogenous ketone dosage (K) in step (b) is directly proportional to the measured impaired glucose utilization.

In certain embodiments, the method further comprises concurrently administering at least one standard-of-care therapy for the glucose hypometabolic disorder, wherein the exogenous ketone composition provides an unexpectedly synergistic improvement in clinical or functional outcomes relative to the standard-of-care therapy alone. In some embodiments, the standard-of-care therapy is a standard-of-care-therapy for Alzheimer's disease. In some embodiments, the standard-of-care therapy is a standard-of-care-therapy for Parkinson's disease. In some embodiments, the standard-of-care therapy is a standard-of-care-therapy for heart failure.

In certain embodiments, the exogenous ketone therapy is administered in combination with at least one standard-of-care treatment for the subject's condition. For example, a subject with Alzheimer's disease may continue on an acetylcholinesterase inhibitor or other approved Alzheimer's medication while receiving the ketone therapy; a subject with Parkinson's disease may concurrently receive dopaminergic therapy (e.g., L-DOPA or a dopamine agonist); and a heart failure patient may remain on standard heart failure medications such as beta blockers or ACE inhibitors. The ketone supplementation in these cases provides a synergistic or additive improvement in clinical outcomes relative to standard therapy alone, by addressing the metabolic deficit underlying the disease. In essence, the platform can augment existing therapies rather than replace them, integrating metabolic treatment into the patient's overall care regimen.

In certain embodiments, the exogenous ketone composition comprises delivering an amount sufficient to raise the subject's blood beta-hydroxybutyrate (βHB) concentration to at least about 0.5 millimolar (mM), wherein a therapeutic level of ketosis is induced thereby alleviating the measured glucose metabolism deficit (G_Diff).

In certain embodiments, the exogenous ketone composition is administered via any suitable route, including but not limited to oral, enteral (e.g., through a feeding tube), parenteral (such as intravenous infusion), or other routes tailored for efficient delivery. For example, the ketone formulation can be given sublingually or buccally (e.g., as a dissolvable film, lozenge, or spray) to allow rapid absorption through the oral mucosa. In other embodiments, a transdermal patch or implant provides slow, sustained release of a ketone precursor, ensuring prolonged therapeutic ketosis without repeated dosing. Yet another delivery mode involves intranasal or inhalable formulations—for instance, an aerosolized ketone preparation that can be inhaled for direct uptake via the lungs. These alternative delivery routes, in addition to standard oral or injectable routes, broaden the clinical usability of the therapy and can be selected based on the patient's needs (rapid vs. sustained release, outpatient vs. inpatient setting, etc.).

The exogenous ketone composition itself can take on various forms. In different embodiments, the composition comprises one or more of the following (alone or in combination): β-hydroxybutyrate (βHB) in a salt form (with a physiologically acceptable cation) or attached to a carrier molecule; acetoacetate (AcAc) in salt or ester form; ketone esters (for example, glycerol tri-β-hydroxybutyrate, 1,3-butanediol mono- or di-ester of βHB, or other polyol-based ketone esters as described elsewhere herein); ketone amides or ethers; medium-chain triglycerides (MCTs) that are metabolized into ketone bodies; and various ketone analogs or precursors that yield ketone bodies upon metabolism (such as 1,3-butanediol itself, or certain ketogenic amino acids). Any pharmaceutically acceptable derivative of these compounds (e.g., salts, esters, or co-crystals) is included. In some embodiments, the chosen ketone composition is one of the specifically disclosed compounds of Formula (A) (for instance, a polyol bearing multiple β-hydroxybutyrate groups) formulated with a suitable excipient. In all cases, the administered exogenous ketone composition is provided in an amount effective to achieve a therapeutically useful level of ketosis (for example, raising blood βHB to around or above 0.5 mM), thereby at least partially compensating for the subject's measured glucose metabolism deficit.

In certain embodiments, the exogenous ketone composition is administered orally, enterally, parenterally, or intravenously, and the exogenous ketone composition is selected from the group consisting of: beta-hydroxybutyrate (βHB) salts, beta-hydroxybutyrate (βHB) bound to any carrier molecule, acetoacetate (AcAc) salts, acetoacetate (AcAc) bound to any carrier molecule, 1,3-butanediol, glycerol tributyrate or other glycerol-butyric acid esters, medium-chain triglycerides (MCTs) capable of conversion to ketone bodies, ketone amides, ketone ethers, ketone analogs, and any pharmaceutically acceptable derivatives or combinations thereof, and wherein the administered exogenous ketone composition provides a therapeutically effective level of ketones that at least partially compensates for the subject's measured glucose metabolism deficit. In some embodiments, the exogenous ketone composition is an exogenous ketone composition described herein (e.g., in the Compounds and Compositions section).

In certain embodiments, the exogenous ketone composition is a compound of Formula (A):

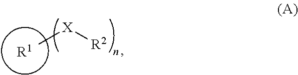

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

. In certain embodiments, the exogenous ketone composition is a compound in Table 1. In certain embodiments, the exogenous ketone composition is a compound selected from the group consisting of:

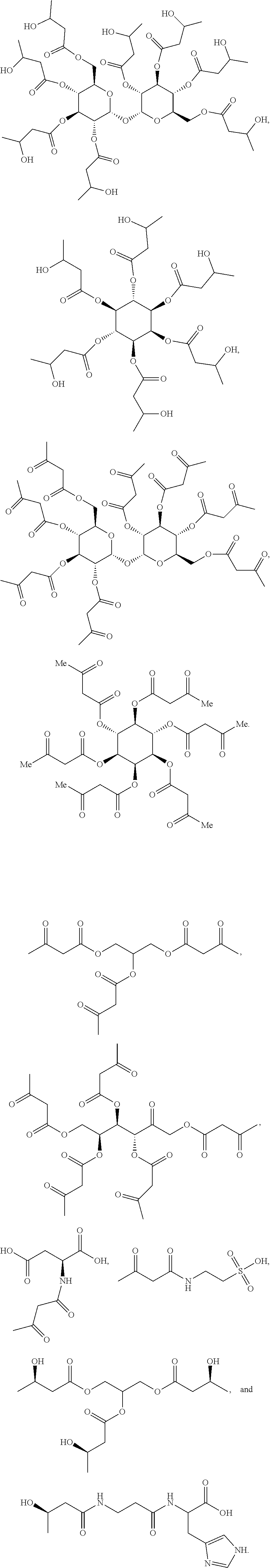

Another aspect of the disclosure provides a method of treating a subject diagnosed with a glucose hypometabolic disorder, comprising:
(a) administering to the subject an exogenous ketone composition in an amount sufficient to achieve and maintain a sustained blood ketone level of at least about 0.5 mM (3-hydroxybutyrate (βHB) for a therapeutically effective duration; and
(b) periodically measuring or estimating the subject's ketone levels by one or more suitable techniques,
wherein said sustained blood ketone level alleviates or improves one or more clinical symptoms of the glucose hypometabolic disorder, independent of explicitly measuring any glucose metabolism deficit in the subject.

In certain embodiments, the glucose hypometabolic disorder is selected from the group consisting of Alzheimer's disease, Parkinson's disease, heart failure, type 2 diabetes, epilepsy, traumatic brain injury, amyotrophic lateral sclerosis, cancer cachexia, metabolic syndrome, and any condition in which the subject's tissue exhibits a quantifiable reduction in glucose utilization. In certain embodiments, the glucose hypometabolic disorder is Alzheimer's disease. In certain embodiments, the glucose hypometabolic disorder is Parkinson's disease. In certain embodiments, the glucose hypometabolic disorder is heart failure.

In certain embodiments, the exogenous ketone composition comprises a sustained-release or slow-release formulation, thereby maintaining the blood ketone level at or above about 0.5 mM for at least about 4 hours following a single administration.

In certain embodiments, the periodic measurement or estimation of the subject's ketone levels is performed using any invasive or non-invasive technique, including but not limited to finger-stick blood testing, continuous or intermittent ketone monitoring devices, breath ketone analysis, transdermal sensors, saliva testing, or urine ketone measurements.

In certain embodiments, the method further comprises adjusting a dosage or frequency of administration of the exogenous ketone composition based on said measured or estimated ketone levels, thereby maintaining a therapeutic blood ketone concentration between about 0.5 mM and about 5.0 mM.

In certain embodiments, the method further comprises administering, concurrently or sequentially, at least one adjunctive metabolic therapy selected from lactate, pyruvate, medium-chain triglycerides (MCTs), insulin-sensitizing agents, or hyperbaric oxygen therapy, wherein said adjunctive therapy enhances or complements the therapeutic efficacy of sustained ketosis.

In certain embodiments, the ketone therapy is combined with the administration of one or more additional metabolic fuels or modulators to further support cellular energy production. For instance, lactate or pyruvate can be administered alongside the exogenous ketones, either intravenously or via another route, providing an alternative fuel that certain tissues can utilize directly. In other embodiments, an insulin-sensitizing agent (such as metformin or a PPAR agonist) is co-administered to improve the efficiency of the subject's residual glucose utilization and enhance uptake of ketones by tissues. These adjunctive treatments, when used in conjunction with the ketone supplementation, help to alleviate the energy crisis by attacking the problem from multiple angles—e.g., improving glucose handling with insulin sensitizers or supplying additional fuel substrates like lactate. The combination of ketones with such co-therapies is intended to be within the scope of the present platform, as it addresses the same underlying glucose hypometabolism with a multi-faceted approach.

In certain embodiments, the exogenous ketone composition is administered via any suitable route, including but not limited to oral, enteral (e.g., through a feeding tube), parenteral (such as intravenous infusion), or other routes tailored for efficient delivery. For example, the ketone formulation can be given sublingually or buccally (e.g., as a dissolvable film, lozenge, or spray) to allow rapid absorption through the oral mucosa. In other embodiments, a transdermal patch or implant provides slow, sustained release of a ketone precursor, ensuring prolonged therapeutic ketosis without repeated dosing. Yet another delivery mode involves intranasal or inhalable formulations—for instance, an aerosolized ketone preparation that can be inhaled for direct uptake via the lungs. These alternative delivery routes, in addition to standard oral or injectable routes, broaden the clinical usability of the therapy and can be selected based on the patient's needs (rapid vs. sustained release, outpatient vs. inpatient setting, etc.).

The exogenous ketone composition itself can take on various forms. In different embodiments, the composition comprises one or more of the following (alone or in combination): β-hydroxybutyrate (βHB) in a salt form (with a physiologically acceptable cation) or attached to a carrier molecule; acetoacetate (AcAc) in salt or ester form; ketone esters (for example, glycerol tri-β-hydroxybutyrate, 1,3-butanediol mono- or di-ester of βHB, or other polyol-based ketone esters as described elsewhere herein); ketone amides or ethers; medium-chain triglycerides (MCTs) that are metabolized into ketone bodies; and various ketone analogs or precursors that yield ketone bodies upon metabolism (such as 1,3-butanediol itself, or certain ketogenic amino acids). Any pharmaceutically acceptable derivative of these compounds (e.g., salts, esters, or co-crystals) is included. In some embodiments, the chosen ketone composition is one of the specifically disclosed compounds of Formula (A) (for instance, a polyol bearing multiple β-hydroxybutyrate groups) formulated with a suitable excipient. In all cases, the administered exogenous ketone composition is provided in an amount effective to achieve a therapeutically useful level of ketosis (for example, raising blood βHB to around or above 0.5 mM), thereby at least partially compensating for the subject's measured glucose metabolism deficit.

In certain embodiments, the exogenous ketone composition is administered orally, enterally, parenterally, or intravenously, and the exogenous ketone composition is selected from the group consisting of: beta-hydroxybutyrate (βHB) salts, beta-hydroxybutyrate (βHB) bound to any carrier molecule, acetoacetate (AcAc) salts, acetoacetate (AcAc) bound to any carrier molecule, 1,3-butanediol, glycerol tributyrate or other glycerol-butyric acid esters, medium-chain triglycerides (MCTs) capable of conversion to ketone bodies, ketone amides, ketone ethers, ketone analogs, and any pharmaceutically acceptable derivatives or combinations thereof, and wherein the administered exogenous ketone composition provides a therapeutically effective level of ketones that at least partially compensates for the subject's measured glucose metabolism deficit. In some embodiments, the exogenous ketone composition is an exogenous ketone composition described herein (e.g., in the Compounds and Compositions section).

In certain embodiments, the exogenous ketone composition is a compound of Formula (A):

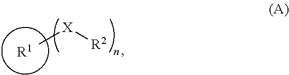

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

In certain embodiments, the exogenous ketone composition is a compound in Table 1.

In certain embodiments, the exogenous ketone composition is a compound selected from the group consisting of

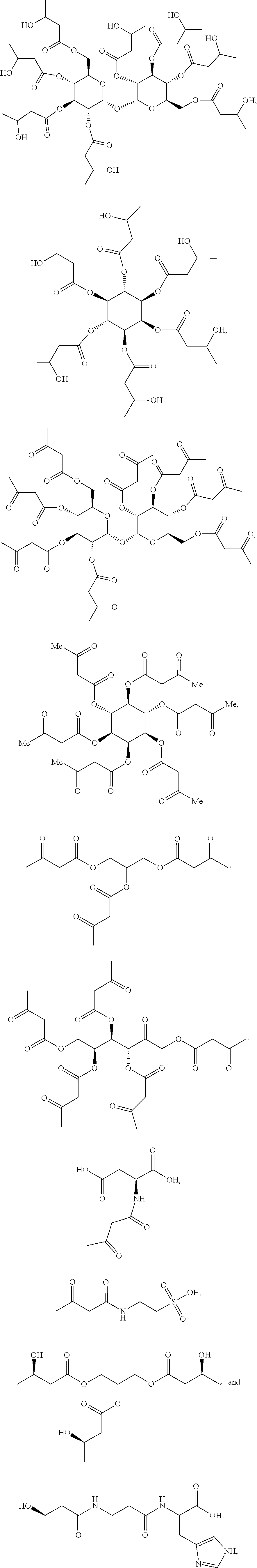

### Hyperbaric Oxygen

Another aspect of the disclosure provides a method of treating a subject diagnosed with a glucose hypometabolic disorder, comprising:
(a) measuring a glucose metabolism deficit (G_Diff) in one or more tissues of the subject using at least one diagnostic technique;
(b) calculating a personalized exogenous ketone dosage (K) based on the measured G_Diff using Formula (I)

$$K = G\_Diff \times ( 4 / 4.7 ) \times F \times p , \qquad ( I )$$

wherein F is an efficiency factor and p is a partition factor representing tissue-specific ketone uptake capacity; and
(c) administering an exogenous ketone composition to the subject in accordance with the calculated dosage, wherein the administered exogenous ketone composition provides an alternative metabolic fuel that compensates for the subject's glucose hypometabolism.

It will be appreciated that Formula (I) is one exemplary approach to determining the ketone dosage K based on G_Diff. In other embodiments, the personalized ketone dosage can be calculated using different algorithms or computational techniques. For instance, a machine-learning model (trained on data from numerous patients) may predict an optimal ketone dose by taking the measured G_Diff as well as additional patient-specific parameters into account (such as the patient's age, liver function, current diet, or oxygen saturation). In another embodiment, a simple lookup table or an iterative titration protocol is used: the system might start at a baseline ketone dose and adjust upward or downward in controlled increments until the patient's metabolic markers reach target ranges. The computational module performing the dose calculation can be implemented locally (e.g., on a wearable device or pump controller) or remotely (e.g., a cloud server that receives the diagnostic data and returns a dosing recommendation). In all cases, the core principle is that the dose is quantitatively based on the measured glucose deficit, regardless of the specific mathematical model used. This ensures that even if different formulae or algorithms are employed, they fall within the scope of using G_Diff to inform therapy.

In certain embodiments, the method further comprises optionally administering hyperbaric oxygen therapy (HBOT) to the subject at a pressure of between 1.5 and 3.0 atmospheres absolute (ATA), wherein the combined administration of exogenous ketones and HBOT may produce enhanced cellular energy production by simultaneously addressing both fuel substrate deficiency and oxygen availability.

In certain embodiments, the glucose hypometabolic disorder is selected from the group consisting of Alzheimer's disease, mild cognitive impairment, Parkinson's disease, heart failure, type 2 diabetes, epilepsy, traumatic brain injury, amyotrophic lateral sclerosis, cancer cachexia, metabolic syndrome, and any condition in which the subject's tissue exhibits a quantifiable reduction in glucose utilization. In certain embodiments, the glucose hypometabolic disorder is Alzheimer's disease. In certain embodiments, the glucose hypometabolic disorder is Parkinson's disease. In certain embodiments, the glucose hypometabolic disorder is heart failure.

In certain embodiments, the method further comprises classifying the subject's glucose hypometabolic disorder into one of the following metabolic stages based on the measured G_Diff:
(a) Stage 0 corresponding to normal glucose metabolism;
(b) Stage 1 corresponding to mild hypometabolism with a G_Diff of 5-20% below normal;
(c) Stage 2 corresponding to moderate hypometabolism with a G_Diff of 20-40% below normal;
(d) Stage 3 corresponding to severe hypometabolism with a G_Diff of 40-70% below normal; or
(e) Stage 4 corresponding to extreme hypometabolism with a G_Diff greater than 70% below normal, wherein both the optional HBOT parameters and the calculated ketone dosage may be progressively adjusted based on the metabolic stage.

In certain embodiments, for subjects classified in more advanced metabolic stages (Stage 3-4), the optional HBOT is administered according to parameters adjusted to the severity of glucose hypometabolism.

In certain embodiments, when HBOT is administered in conjunction with ketone therapy, the efficiency factor (F) in the ketone dosage calculation formula may be adjusted to account for the potential synergistic effects of combined therapy.

### Enhanced Diagnostic Methods

Another aspect of the disclosure provides a method for diagnosing and quantifying glucose hypometabolism in a subject, comprising:
(a) performing an FDG-PET scan to measure regional glucose uptake;
(b) performing magnetic resonance spectroscopy to measure lactate/pyruvate ratios in the same regions;
(c) measuring at least three blood biomarkers selected from insulin, glycated albumin, fructosamine, pyruvate, lactate, and glucose variability indices; and
(d) integrating data from steps (a), (b), and (c) using a machine learning algorithm to calculate a comprehensive Glucose Metabolism Index (GMI) that quantifies the subject's tissue-specific and global glucose utilization capacity, wherein the calculated GMI provides a more accurate basis for ketone dosage calculation than any single measurement alone.

Another aspect of the disclosure provides a method for non-invasively diagnosing glucose hypometabolism in a subject, comprising:
(a) measuring a panel of at least five biomarkers in the subject's blood, wherein said biomarkers comprise glucose, insulin, lactate, pyruvate, and at least one inflammatory marker;
(b) measuring the subject's exhaled breath for volatile organic compounds associated with altered glucose metabolism;
(c) collecting continuous glucose monitoring data for at least 72 hours; and
(d) applying a diagnostic algorithm to the collected data to diagnose the presence and severity of glucose hypometabolism, wherein the diagnosis enables ketone therapy without requiring more invasive or expensive diagnostic procedures.

In certain embodiments, the glucose metabolism deficit is assessed indirectly by leveraging surrogate markers or alternative imaging techniques. For instance, functional MRI (fMRI) or arterial spin labeling may be used to evaluate tissue perfusion and infer metabolic activity, and near-infrared spectroscopy (NIRS) can monitor regional oxygenation as a proxy for metabolism. In other embodiments, non-invasive physiological indicators are measured—such as the subject's breath acetone levels, respiratory quotient (RQ), or changes in blood lactate during a glucose tolerance test—which correlate with cellular metabolic state. These surrogate data are then correlated to known glucose utilization rates (for example, via a machine-learning model or lookup table) to estimate G_Diff without requiring direct PET or similar imaging. Such approaches enable diagnosis of glucose hypometabolism using readily obtainable metrics, expanding the toolkit beyond traditional imaging.

### Patient Stratification

Another aspect of the disclosure provides a method of stratifying subjects for personalized ketone therapy, comprising:
(a) determining the subject's genotype for at least three genetic markers selected from APOE, PPAR-gamma, MCT1, BDH1, SCOT, and HMGCS2;
(b) measuring the subject's baseline ketone metabolism by administering a test dose of exogenous ketones and measuring blood ketone levels at predetermined intervals;
(c) calculating a Ketone Responsiveness Index (KRI) based on the genetic profile and metabolic test results; and
(d) stratifying the subject into one of at least three ketone therapy protocol groups based on the calculated KRI, wherein each protocol group is associated with a different dosing regimen, formulation type, or administration schedule to optimize therapeutic outcomes.

Another aspect of the disclosure provides a method of stratifying subjects based on metabolic phenotype for ketone therapy, comprising:
(a) measuring the subject's insulin sensitivity using a glucose tolerance test;
(b) measuring the subject's baseline ketone production capacity using a controlled fast;
(c) assessing the subject's mitochondrial function using a standardized exercise capacity test; and
(d) classifying the subject into one of at least four metabolic phenotypes based on the measured parameters, wherein the metabolic phenotype classification determines the subject's ketone therapy protocol, including dosage, timing, and formulation.

### Response Prediction Algorithms

Another aspect of the disclosure provides a method of predicting a subject's response to ketone therapy, comprising:
(a) collecting at least ten data parameters from the subject, wherein said parameters include age, disease type, disease duration, glucose metabolism measurements, inflammatory markers, genetic markers, and baseline cognitive or physical performance metrics;
(b) inputting the collected parameters into a trained machine learning model that has been developed using data from at least 100 previous ketone therapy recipients; and
(c) generating a predicted response score that indicates the likelihood of the subject achieving at least a 30% improvement in at least one primary outcome measure, wherein the predicted response score guides therapy decisions and personalized dosing strategies.

Another aspect of the disclosure provides a method of predicting and optimizing a subject's response to ketone therapy over time, comprising:
(a) establishing baseline measurements for the subject, including glucose metabolism, ketone metabolism, and clinical symptoms;
(b) initiating ketone therapy according to an initial dosing protocol;
(c) collecting response data at predetermined intervals during treatment;
(d) inputting the response data into a reinforcement learning algorithm that dynamically updates the predicted optimal dosing protocol; and
(e) adjusting the ketone therapy based on the algorithm's recommendations, wherein the dynamic response algorithm improves therapeutic outcomes by at least 20% compared to static dosing protocols.

This at least 20% improvement arises because the algorithm continually adapts the dosing to the individual's response, thereby fine-tuning therapy in a way static protocols cannot.

### Therapeutic Monitoring

Another aspect of the disclosure provides a method of monitoring and optimizing ketone therapy in a subject, comprising:
(a) utilizing a wearable device that continuously monitors at least three parameters selected from glucose levels, ketone levels, heart rate variability, and physical activity;
(b) transmitting the monitored data to a computational system that applies an algorithm to assess therapeutic adequacy;
(c) generating alerts when measured parameters fall outside target ranges; and
(d) providing automated dosing recommendations to maintain optimal therapeutic levels, wherein the continuous monitoring system improves patient adherence and clinical outcomes by at least 25% compared to periodic monitoring (by enabling more timely adjustments and tighter control of metabolic parameters).

Another aspect of the disclosure provides a method of monitoring response to ketone therapy, comprising
(a) measuring a panel of biomarkers before initiating ketone therapy to establish baseline values;
(b) re-measuring the same biomarker panel at predetermined intervals during ketone therapy;
(c) calculating a Treatment Response Index (TRI) based on changes in the biomarker panel; and
(d) adjusting the ketone therapy protocol based on the calculated TRI, wherein the biomarker panel comprises markers of inflammation, oxidative stress, mitochondrial function, and glucose-ketone metabolism, and wherein improvements in the TRI correlate with clinical improvement.

### Digital Therapeutic

Another aspect of the disclosure provides a method of enhancing ketone therapy efficacy, comprising:
(a) administering exogenous ketones according to a personalized dosage protocol;
(b) providing the subject with a digital therapeutic application that delivers behavioral interventions synchronized with the ketone dosing schedule;
(c) collecting adherence and symptom data through the digital therapeutic application; and
(d) adjusting the ketone therapy based on the collected data, wherein the combined physical-digital therapeutic approach improves clinical outcomes by at least 35% compared to ketone therapy without the digital component (for example, by improving patient adherence through reminders, optimizing dosing schedules based on behavioral data, and providing feedback that keeps patients engaged with the therapy).

Another aspect of the disclosure provides a method of optimizing ketone therapy across a population, comprising:
(a) collecting anonymized treatment and outcome data from multiple subjects receiving personalized ketone therapy;
(b) analyzing the collected data using a machine learning algorithm to identify patterns associated with optimal responses;
(c) generating updated treatment protocols based on the identified patterns; and
(d) implementing the updated protocols for new subjects, wherein the iterative optimization process continuously improves treatment efficacy and reduces adverse effects across the treated population.

### Systems

Disclosed herein, in certain embodiments, are systems for diagnosing and treating a subject diagnosed with a glucose hypometabolic disorder (e.g., Alzheimer's disease, Parkinson's disease, or heart failure).

### Quantitative Diagnostic Component

Multi-modal glucose metabolism assessment: The system employs a combination of diagnostic technologies (PET, MRS, metabolic assays, wearable biosensors) to precisely quantify tissue-specific glucose metabolism deficits (G_Diff).

Novel biomarker panel: The invention identifies and utilizes a panel of specific biomarkers that correlate with glucose hypometabolism across conditions.

Proprietary algorithm: A computational algorithm integrates multiple data inputs to calculate a standardized Glucose Metabolism Index (GMI) that quantifies the magnitude of hypometabolism.

### Precision Therapeutic Component

Personalized dosing algorithm: The system calculates an individualized ketone dosage (K) based on the measured glucose deficit using the novel Formula (I):

$$K = G\_Diff \times ( 4 / 4.7 ) \times F \times p \qquad ( I )$$

wherein:
   G_Diff is the measured glucose deficit
   (4/4.7) accounts for relative energy yields of glucose versus ketones
   F is an efficiency factor reflecting disease-specific factors
   p is a partition factor representing tissue-specific ketone uptake capacity

Customized ketone formulations: The system provides specific ketone formulations optimized for different conditions and individual metabolic profiles.

It will be appreciated that Formula (I) is one exemplary approach to determining the ketone dosage K based on G_Diff. In other embodiments, the personalized ketone dosage can be calculated using different algorithms or computational techniques. For instance, a machine-learning model (trained on data from numerous patients) may predict an optimal ketone dose by taking the measured G_Diff as well as additional patient-specific parameters into account (such as the patient's age, liver function, current diet, or oxygen saturation). In another embodiment, a simple lookup table or an iterative titration protocol is used: the system might start at a baseline ketone dose and adjust upward or downward in controlled increments until the patient's metabolic markers reach target ranges. The computational module performing the dose calculation can be implemented locally (e.g., on a wearable device or pump controller) or remotely (e.g., a cloud server that receives the diagnostic data and returns a dosing recommendation). In all cases, the core principle is that the dose is quantitatively based on the measured glucose deficit, regardless of the specific mathematical model used. This ensures that even if different formulae or algorithms are employed, they fall within the scope of using G_Diff to inform therapy.

Dynamic adjustment protocol: The therapy includes ongoing monitoring and algorithmic adjustment of ketone dosing based on clinical response and metabolic parameters.

### Integrated System Advantages

Mechanism-based approach: Unlike symptom-focused therapies, this system addresses the underlying metabolic pathophysiology common across conditions.

Cross-disease applicability: The same core diagnostic-therapeutic platform can be applied across traditionally separate disease categories.

Objective, quantifiable metrics: Treatment is guided by measurable metabolic parameters rather than subjective assessments.

Synergistic with existing therapies: The platform can be combined with standard-of-care treatments to achieve enhanced outcomes.

### System for Diagnosing and Treating a Subject

One aspect of the disclosure provides a system for diagnosing and treating a glucose hypometabolic disorder in a subject, comprising:
(a) a diagnostic module configured to measure a glucose metabolism deficit (G_Diff) in the subject's tissue using at least one diagnostic technique;
(b) a computational module, operably connected to the diagnostic module, that applies an algorithm converting the measured G_Diff into a recommended exogenous ketone dosage (K) to compensate for the subject's impaired glucose utilization; and
(c) a ketone delivery apparatus configured to administer the recommended ketone dosage to the subject,
wherein the diagnostic module, computational module, and ketone delivery apparatus together provide a unified diagnostic-therapeutic platform to treat the glucose hypometabolic disorder by supplying ketones in proportion to the measured deficit.

In certain embodiments, the computational module calculates the personalized exogenous ketone dosage (K) using Formula (I):

$$K = G\_Diff \times ( 4 / 4.7 ) \times F \times p \qquad ( I )$$

wherein:
G_Diff is the measured glucose deficit in grams or equivalent energy units;
(4/4.7) is a factor accounting for relative energy yields of glucose versus ketones;
F is an efficiency factor (F≥1) reflecting systemic overhead or disease-specific factors; and p is a partition factor (0<p≤1) representing tissue-specific ketone uptake capacity.

In certain embodiments, the at least one diagnostic technique is selected from positron emission tomography (PET), magnetic resonance spectroscopy (MRS), metabolic assays, and wearable biosensors.

In certain embodiments, the ketone delivery apparatus comprises an automated infusion pump that dynamically adjusts the exogenous ketone flow rate based on real-time feedback of the subject's glucose or ketone levels, maintaining a target therapeutic ketone range that partially or fully offsets the measured G_Diff.

In some implementations, the system operates in a fully automated, closed-loop manner—for instance, an implanted or wearable pump adjusts ketone infusion in real time based on sensor inputs, without user intervention. In other implementations, the system functions in a semi-automated advisory mode: the diagnostic and computational modules generate a recommended ketone dosage or adjustment, which is then presented to a caregiver or the patient (e.g., via a user interface or smartphone app) instead of being automatically enacted. In this advisory scenario, the output from the computational module can be delivered to external devices or software—for example, sent as a dosing recommendation to a clinician's computer, displayed on the patient's app, or even transmitted to a third-party infusion pump's control system via an API—allowing a human to confirm and manually administer the dose. Both approaches (fully automatic and advisory) leverage the same quantified-deficit input and dosing algorithm; the distinction lies in whether the final control action is manual or automatic. Accordingly, the term “system” as used herein encompasses setups that provide dosage guidance for user implementation as well as those that directly control ketone delivery.

The modular nature of the system allows the diagnostic, computational, and delivery components to be geographically or physically separated as long as they are operably connected (e.g., via wired or wireless communication). For instance, the diagnostic module might be a hospital's existing PET scanner or a wearable continuous glucose monitor that sends metabolic data to the computational module over a network. The computational module could be a cloud-based server that receives the input data remotely, performs the dosage calculation, and then transmits the recommended dosage to the ketone delivery apparatus. The ketone delivery apparatus in this scenario could be an adaptable infusion pump or an injection device which accepts the dosage instructions (either automatically or through a user intermediary). This interoperability means the platform can integrate with third-party devices and software. In one embodiment, the system's software outputs the dose instruction in a standardized format or via an API, enabling integration into electronic health record systems or direct interfacing with an infusion pump's control software. Such configurations illustrate that the invention can be implemented using a combination of existing medical infrastructure and new computational tools, rather than a single monolithic device.

### Closed-Loop Systems

Another aspect of the disclosure provides a closed-loop system for ketone therapy, comprising:
(a) a continuous monitoring component that measures the subject's blood ketone levels at intervals not exceeding 15 minutes;
(b) a computational component that processes the measured ketone levels and compares them to target ranges determined by the subject's measured glucose metabolism deficit;
(c) a delivery component capable of adjusting ketone administration rates based on signals from the computational component; and
(d) a user interface that displays real-time data and alerts, wherein the closed-loop system maintains blood ketone levels within ±15% of target values for at least 80% of the treatment period.

Another aspect of the disclosure provides a method of implementing closed-loop ketone therapy, comprising:
(a) continuously monitoring the subject's glucose levels, ketone levels, and at least one additional physiological parameter selected from heart rate, blood pressure, and body temperature;
(b) processing the monitored parameters using an algorithm that calculates optimal ketone levels based on current glucose levels and the additional physiological parameter;
(c) automatically adjusting ketone delivery rates to maintain the calculated optimal ketone levels; and
(d) recording all data for subsequent efficacy analysis, wherein the multi-parameter monitoring enables more precise therapeutic control than ketone monitoring alone.

### Hyperbaric Oxygen

Another aspect of the disclosure provides a system for treating a glucose hypometabolic disorder in a subject, comprising:
(a) a diagnostic module configured to measure a glucose metabolism deficit (G_Diff) in one or more tissues of the subject;
(b) a computational module that calculates a personalized ketone dosage using the formula K=G_Diff×(4/4.7)×F×p;
(c) a ketone delivery apparatus configured to administer the calculated ketone dosage; and
(d) optionally, a hyperbaric oxygen chamber configured to deliver oxygen at an elevated pressure determined based on the measured G_Diff, wherein the system provides treatment that addresses the fuel component of glucose hypometabolism, with optional enhancement through oxygen therapy.

### Kits

Disclosed herein, in certain embodiments, are kits for diagnosing and treating a subject diagnosed with a glucose hypometabolic disorder (e.g., Alzheimer's disease, Parkinson's disease, or heart failure).

One aspect of the invention provides a kit for diagnosing and treating a glucose hypometabolic disorder in a subject, comprising:
(a) instructions for measuring or estimating a glucose metabolism deficit (G_Diff) in the subject using at least one diagnostic technique;
(b) an exogenous ketone composition suitable for oral or parenteral administration; and
(c) a reference chart or algorithmic guide that translates the measured G_Diff into a recommended personalized exogenous ketone dosage for mitigating the subject's impaired glucose utilization, wherein the kit enables a practitioner or patient to determine a personalized exogenous ketone dosage that compensates for the subject's impaired glucose utilization.

In certain embodiments, the at least one diagnostic technique is selected from positron emission tomography (PET), magnetic resonance spectroscopy (MRS), metabolic assays, and wearable biosensors.

In certain embodiments, said reference chart or algorithmic guide includes guidance on applying a partition factor (p) representing tissue-specific ketone uptake capacity, thereby adjusting the recommended ketone dosage to match the subject's particular organ demands or disease severity.

In certain embodiments, the reference chart or algorithmic guide comprises computing the personalized exogenous ketone dosage (K) using Formula (I):

$$K = G\_Diff \times ( 4 / 4.7 ) \times F \times p \qquad ( I )$$

wherein:
G_Diff is the measured glucose deficit in grams or equivalent energy units;
(4/4.7) is a factor accounting for relative energy yields of glucose versus ketones;
F is an efficiency factor (F≥1) reflecting systemic overhead or disease-specific factors; and p is a partition factor (0<p≤1) representing tissue-specific ketone uptake capacity.

### Hyperbaric Oxygen

Another aspect of the disclosure provides a kit for treating a glucose hypometabolic disorder in a subject, comprising:
(a) an exogenous ketone composition;
(b) instructions for measuring or estimating a glucose metabolism deficit (G_Diff);
(c) a reference guide that translates the measured G_Diff into a recommended ketone dosage using the formula K=G_Diff×(4/4.7)×F×p; and
(d) optionally, an HBOT protocol guide that specifies recommended parameters for optional adjunctive hyperbaric oxygen therapy based on the measured G_Diff and classified metabolic stage, wherein the kit enables implementation of the core ketone therapy with guidance for optional HBOT enhancement.

### Pharmaceutical Compositions and Routes of Administration

Disclosed herein, in certain embodiments, are pharmaceutical compositions comprising: (a) a compound disclosed herein (e.g., a compound of Formula (A) or a pharmaceutically acceptable salt thereof), and (b) a pharmaceutically acceptable excipient. In some embodiments, the excipient is selected from: inert solid diluents and fillers, diluents, including sterile aqueous solution and various organic solvents, permeation enhancers, solubilizers and adjuvants. In some embodiment, the pharmaceutical compositions are administered alone or in combination with other therapeutic agents. Such compositions are prepared in any suitable manner.

In certain embodiments, the pharmaceutical composition comprises at least two unique compounds of Formula (A). In certain embodiments, at least one compound comprises R² as β-hydroxybutyryl and at least one compound comprises R² as acetoacetyl. In certain embodiments, the at least two unique compounds of Formula (A) comprise the same R¹ carrier molecule In certain embodiments, the pharmaceutical composition comprises at least two compounds of Formula (A). In certain embodiments, the comprises at least two compounds from Table 1.

In certain embodiments, the pharmaceutical composition comprises Compound 1 and Compound 2. In certain embodiments, the pharmaceutical composition comprises Compound 1 and Compound 3. In certain embodiments, the pharmaceutical composition comprises Compound 1 and Compound 4. In certain embodiments, the pharmaceutical composition comprises Compound 1 and Compound 5. In certain embodiments, the pharmaceutical composition comprises Compound 1 and Compound 6. In certain embodiments, the pharmaceutical composition comprises Compound 1 and Compound 7. In certain embodiments, the pharmaceutical composition comprises Compound 1 and Compound 8. In certain embodiments, the pharmaceutical composition comprises Compound 1 and Compound 9. In certain embodiments, the pharmaceutical composition comprises Compound 1 and Compound 10. In certain embodiments, the pharmaceutical composition comprises Compound 1 and Compound 11. In certain embodiments, the pharmaceutical composition comprises Compound 1 and Compound 12. In certain embodiments, the pharmaceutical composition comprises Compound 1 and Compound 13. In certain embodiments, the pharmaceutical composition comprises Compound 1 and Compound 14. In certain embodiments, the pharmaceutical composition comprises Compound 1 and Compound 15.

In certain embodiments, the pharmaceutical composition comprises Compound 2 and Compound 3. In certain embodiments, the pharmaceutical composition comprises Compound 2 and Compound 4. In certain embodiments, the pharmaceutical composition comprises Compound 2 and Compound 5. In certain embodiments, the pharmaceutical composition comprises Compound 2 and Compound 6. In certain embodiments, the pharmaceutical composition comprises Compound 2 and Compound 7. In certain embodiments, the pharmaceutical composition comprises Compound 2 and Compound 8. In certain embodiments, the pharmaceutical composition comprises Compound 2 and Compound 9. In certain embodiments, the pharmaceutical composition comprises Compound 2 and Compound 10. In certain embodiments, the pharmaceutical composition comprises Compound 2 and Compound 11. In certain embodiments, the pharmaceutical composition comprises Compound 2 and Compound 12. In certain embodiments, the pharmaceutical composition comprises Compound 2 and Compound 13. In certain embodiments, the pharmaceutical composition comprises Compound 2 and Compound 14. In certain embodiments, the pharmaceutical composition comprises Compound 2 and Compound 15.

In certain embodiments, the pharmaceutical composition comprises Compound 3 and Compound 4. In certain embodiments, the pharmaceutical composition comprises Compound 3 and Compound 5. In certain embodiments, the pharmaceutical composition comprises Compound 3 and Compound 6. In certain embodiments, the pharmaceutical composition comprises Compound 3 and Compound 7. In certain embodiments, the pharmaceutical composition comprises Compound 3 and Compound 8. In certain embodiments, the pharmaceutical composition comprises Compound 3 and Compound 9. In certain embodiments, the pharmaceutical composition comprises Compound 3 and Compound 10. In certain embodiments, the pharmaceutical composition comprises Compound 3 and Compound 11. In certain embodiments, the pharmaceutical composition comprises Compound 3 and Compound 12. In certain embodiments, the pharmaceutical composition comprises Compound 3 and Compound 13. In certain embodiments, the pharmaceutical composition comprises Compound 3 and Compound 14. In certain embodiments, the pharmaceutical composition comprises Compound 3 and Compound 15.

In certain embodiments, the pharmaceutical composition comprises Compound 4 and Compound 5. In certain embodiments, the pharmaceutical composition comprises Compound 4 and Compound 6. In certain embodiments, the pharmaceutical composition comprises Compound 4 and Compound 7. In certain embodiments, the pharmaceutical composition comprises Compound 4 and Compound 8. In certain embodiments, the pharmaceutical composition comprises Compound 4 and Compound 9. In certain embodiments, the pharmaceutical composition comprises Compound 4 and Compound 10. In certain embodiments, the pharmaceutical composition comprises Compound 4 and Compound 11. In certain embodiments, the pharmaceutical composition comprises Compound 4 and Compound 12. In certain embodiments, the pharmaceutical composition comprises Compound 4 and Compound 13. In certain embodiments, the pharmaceutical composition comprises Compound 4 and Compound 14. In certain embodiments, the pharmaceutical composition comprises Compound 4 and Compound 15.

In certain embodiments, the pharmaceutical composition comprises Compound 5 and Compound 6. In certain embodiments, the pharmaceutical composition comprises Compound 5 and Compound 7. In certain embodiments, the pharmaceutical composition comprises Compound 5 and Compound 8. In certain embodiments, the pharmaceutical composition comprises Compound 5 and Compound 9. In certain embodiments, the pharmaceutical composition comprises Compound 5 and Compound 10. In certain embodiments, the pharmaceutical composition comprises Compound 5 and Compound 11. In certain embodiments, the pharmaceutical composition comprises Compound 5 and Compound 12. In certain embodiments, the pharmaceutical composition comprises Compound 5 and Compound 13. In certain embodiments, the pharmaceutical composition comprises Compound 5 and Compound 14. In certain embodiments, the pharmaceutical composition comprises Compound 5 and Compound 15.

In certain embodiments, the pharmaceutical composition comprises Compound 6 and Compound 7. In certain embodiments, the pharmaceutical composition comprises Compound 6 and Compound 8. In certain embodiments, the pharmaceutical composition comprises Compound 6 and Compound 9. In certain embodiments, the pharmaceutical composition comprises Compound 6 and Compound 10. In certain embodiments, the pharmaceutical composition comprises Compound 6 and Compound 11. In certain embodiments, the pharmaceutical composition comprises Compound 6 and Compound 12. In certain embodiments, the pharmaceutical composition comprises Compound 6 and Compound 13. In certain embodiments, the pharmaceutical composition comprises Compound 6 and Compound 14. In certain embodiments, the pharmaceutical composition comprises Compound 6 and Compound 15.

In certain embodiments, the pharmaceutical composition comprises Compound 7 and Compound 8. In certain embodiments, the pharmaceutical composition comprises Compound 7 and Compound 9. In certain embodiments, the pharmaceutical composition comprises Compound 7 and Compound 10. In certain embodiments, the pharmaceutical composition comprises Compound 7 and Compound 11. In certain embodiments, the pharmaceutical composition comprises Compound 7 and Compound 12. In certain embodiments, the pharmaceutical composition comprises Compound 7 and Compound 13. In certain embodiments, the pharmaceutical composition comprises Compound 7 and Compound 14. In certain embodiments, the pharmaceutical composition comprises Compound 7 and Compound 15.

In certain embodiments, the pharmaceutical composition comprises Compound 8 and Compound 9. In certain embodiments, the pharmaceutical composition comprises Compound 8 and Compound 10. In certain embodiments, the pharmaceutical composition comprises Compound 8 and Compound 11. In certain embodiments, the pharmaceutical composition comprises Compound 8 and Compound 12. In certain embodiments, the pharmaceutical composition comprises Compound 8 and Compound 13. In certain embodiments, the pharmaceutical composition comprises Compound 8 and Compound 14. In certain embodiments, the pharmaceutical composition comprises Compound 8 and Compound 15.

In certain embodiments, the pharmaceutical composition comprises Compound 9 and Compound 10. In certain embodiments, the pharmaceutical composition comprises Compound 9 and Compound 11. In certain embodiments, the pharmaceutical composition comprises Compound 9 and Compound 12. In certain embodiments, the pharmaceutical composition comprises Compound 9 and Compound 13. In certain embodiments, the pharmaceutical composition comprises Compound 9 and Compound 14. In certain embodiments, the pharmaceutical composition comprises Compound 9 and Compound 15.

In certain embodiments, the pharmaceutical composition comprises Compound 10 and Compound 11. In certain embodiments, the pharmaceutical composition comprises Compound 10 and Compound 12. In certain embodiments, the pharmaceutical composition comprises Compound 10 and Compound 13. In certain embodiments, the pharmaceutical composition comprises Compound 10 and Compound 14. In certain embodiments, the pharmaceutical composition comprises Compound 10 and Compound 15.

In certain embodiments, the pharmaceutical composition comprises Compound 11 and Compound 12. In certain embodiments, the pharmaceutical composition comprises Compound 11 and Compound 13. In certain embodiments, the pharmaceutical composition comprises Compound 11 and Compound 14. In certain embodiments, the pharmaceutical composition comprises Compound 11 and Compound 15.

In certain embodiments, the pharmaceutical composition comprises Compound 12 and Compound 13. In certain embodiments, the pharmaceutical composition comprises Compound 12 and Compound 14. In certain embodiments, the pharmaceutical composition comprises Compound 12 and Compound 15.

In certain embodiments, the pharmaceutical composition comprises Compound 13 and Compound 14. In certain embodiments, the pharmaceutical composition comprises Compound 13 and Compound 15.

In certain embodiments, the pharmaceutical composition comprises Compound 14 and Compound 15.

In certain embodiments, each compound of Formula (A) has the same R¹ and X.

As generally defined above R¹ is a polyol, amino acid, or a derivative thereof. In certain embodiments, R¹ is a polyol. A polyol is a compound containing two or more hydroxy (OH) groups.

In certain embodiments, R¹ is a polyol which is capable of delivering about 1 g to 50 g or more of ketone bodies to a subject diagnosed with a glucose hypometabolic disorder. In certain embodiments, R¹ is a polyol which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a polyol which is capable of delivering about 1 g to 5 g or more of ketone bodies to a subject diagnosed with a glucose hypometabolic disorder. In certain embodiments, R¹ is a polyol which is capable of delivering about 5 g to 10 g or more of ketone bodies to a subject diagnosed with a glucose hypometabolic disorder. In certain embodiments, R¹ is a polyol which is capable of delivering about 10 g to 15 g or more of ketone bodies to a subject diagnosed with a glucose hypometabolic disorder. In certain embodiments, R¹ is a polyol which is capable of delivering about 15 g to 20 g or more of ketone bodies to a subject diagnosed with a glucose hypometabolic disorder. In certain embodiments, R¹ is a polyol which is capable of delivering about 20 g to 25 g or more of ketone bodies to a subject diagnosed with a glucose hypometabolic disorder. In certain embodiments, R¹ is a polyol which is capable of delivering about 25 g to 30 g or more of ketone bodies to a subject diagnosed with a glucose hypometabolic disorder. In certain embodiments, R¹ is a polyol which is capable of delivering about 30 g to about 35 g or more of ketone bodies to a subject diagnosed with a glucose hypometabolic disorder. In certain embodiments, R¹ is a polyol which is capable of delivering about 35 g to 40 g or more of ketone bodies to a subject diagnosed with a glucose hypometabolic disorder. In certain embodiments, R¹ is a polyol which is capable of delivering about 40 g to 45 g or more of ketone bodies to a subject diagnosed with a glucose hypometabolic disorder. In certain embodiments, R¹ is a polyol which is capable of delivering or about 45 g to 50 g or more of ketone bodies to a subject diagnosed with a glucose hypometabolic disorder.

In certain embodiments, R¹ is a polyol which is capable of delivering about 1 g or more of ketone bodies to a subject diagnosed with a glucose hypometabolic disorder. In certain embodiments, R¹ is a polyol which is capable of delivering about 5 g or more of ketone bodies to a subject diagnosed with a glucose hypometabolic disorder. In certain embodiments, R¹ is a polyol which is capable of delivering about 10 g or more of ketone bodies to a subject diagnosed with a glucose hypometabolic disorder. In certain embodiments, R¹ is a polyol which is capable of delivering about 15 g or more of ketone bodies to a subject diagnosed with a glucose hypometabolic disorder. In certain embodiments, R¹ is a polyol which is capable of delivering about 20 g or more of ketone bodies to a subject diagnosed with a glucose hypometabolic disorder. In certain embodiments, R¹ is a polyol which is capable of delivering about 25 g or more of ketone bodies to a subject diagnosed with a glucose hypometabolic disorder. In certain embodiments, R¹ is a polyol which is capable of delivering about 30 g or more of ketone bodies to a subject diagnosed with a glucose hypometabolic disorder. In certain embodiments, R¹ is a polyol which is capable of delivering about 35 g or more of ketone bodies to a subject diagnosed with a glucose hypometabolic disorder. In certain embodiments, R¹ is a polyol which is capable of delivering about 40 g or more of ketone bodies to a subject diagnosed with a glucose hypometabolic disorder. In certain embodiments, R¹ is a polyol which is capable of delivering or about 45 g or more of ketone bodies to a subject diagnosed with a glucose hypometabolic disorder. In certain embodiments, R¹ is a polyol which is capable of delivering or about 50 g or more of ketone bodies to a subject diagnosed with a glucose hypometabolic disorder.

In certain embodiments, R¹ is a carbohydrate. In certain embodiments, R¹ is a carbohydrate which is capable of delivering about 1 g to 50 g or more of ketone bodies to a subject diagnosed with a glucose hypometabolic disorder. In certain embodiments, R¹ is a carbohydrate which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a carbohydrate which is capable of delivering about 1 g to 5 g or more of ketone bodies to a subject diagnosed with a glucose hypometabolic disorder. In certain embodiments, R¹ is a carbohydrate which is capable of delivering about 5 g to 10 g or more of ketone bodies to a subject diagnosed with a glucose hypometabolic disorder. In certain embodiments, R¹ is a carbohydrate which is capable of delivering about 10 g to 15 g or more of ketone bodies to a subject diagnosed with a glucose hypometabolic disorder. In certain embodiments, R¹ is a carbohydrate which is capable of delivering about 15 g to 20 g or more of ketone bodies to a subject diagnosed with a glucose hypometabolic disorder. In certain embodiments, R¹ is a carbohydrate which is capable of delivering about 20 g to 25 g or more of ketone bodies to a subject diagnosed with a glucose hypometabolic disorder. In certain embodiments, R¹ is a carbohydrate which is capable of delivering about 25 g to 30 g or more of ketone bodies to a subject diagnosed with a glucose hypometabolic disorder. In certain embodiments, R¹ is a carbohydrate which is capable of delivering about 30 g to about 35 g or more of ketone bodies to a subject diagnosed with a glucose hypometabolic disorder. In certain embodiments, R¹ is a carbohydrate which is capable of delivering about 35 g to 40 g or more of ketone bodies to a subject diagnosed with a glucose hypometabolic disorder. In certain embodiments, R¹ is a carbohydrate which is capable of delivering about 40 g to 45 g or more of ketone bodies to a subject diagnosed with a glucose hypometabolic disorder. In certain embodiments, R¹ is a carbohydrate which is capable of delivering or about 45 g to 50 g or more of ketone bodies to a subject diagnosed with a glucose hypometabolic disorder.

In certain embodiments, R¹ is a carbohydrate which is capable of delivering about 1 g or more of ketone bodies to a subject diagnosed with a glucose hypometabolic disorder. In certain embodiments, R¹ is a carbohydrate which is capable of delivering about 5 g or more of ketone bodies to a subject diagnosed with a glucose hypometabolic disorder. In certain embodiments, R¹ is a carbohydrate which is capable of delivering about 10 g or more of ketone bodies to a subject diagnosed with a glucose hypometabolic disorder. In certain embodiments, R¹ is a carbohydrate which is capable of delivering about 15 g or more of ketone bodies to a subject diagnosed with a glucose hypometabolic disorder. In certain embodiments, R¹ is an carbohydrate which is capable of delivering about 20 g or more of ketone bodies to a subject diagnosed with a glucose hypometabolic disorder. In certain embodiments, R¹ is a carbohydrate which is capable of delivering about 25 g or more of ketone bodies to a subject diagnosed with a glucose hypometabolic disorder. In certain embodiments, R¹ is an carbohydrate which is capable of delivering about 30 g or more of ketone bodies to a subject diagnosed with a glucose hypometabolic disorder. In certain embodiments, R¹ is a carbohydrate which is capable of delivering about 35 g or more of ketone bodies to a subject diagnosed with a glucose hypometabolic disorder. In certain embodiments, R¹ is a carbohydrate which is capable of delivering about 40 g or more of ketone bodies to a subject diagnosed with a glucose hypometabolic disorder. In certain embodiments, R¹ is a carbohydrate which is capable of delivering or about 45 g or more of ketone bodies to a subject diagnosed with a glucose hypometabolic disorder. In certain embodiments, R¹ is a carbohydrate which is capable of delivering or about 50 g or more of ketone bodies to a subject diagnosed with a glucose hypometabolic disorder.

In certain embodiments, R¹ is an amino acid or a derivative thereof. In certain embodiments, R¹ is a amino acid or a derivative thereof which is capable of delivering about 1 g to 50 g or more of ketone bodies to a subject diagnosed with a glucose hypometabolic disorder. In certain embodiments, R¹ is a amino acid or a derivative thereof which is capable of delivering about 1 g to 5 g, about 5 g to 10 g, about 10 g to 15 g, about 15 g to 20 g, about 20 g to 25 g, about 25 g to 30 g, about 30 g to about 35 g, about 35 g to 40 g, about 40 g to 45 g, or about 45 g to 50 g or more of ketone bodies to a subject diagnosed with a glucose hypometabolic disorder. In certain embodiments, R¹ is a amino acid or a derivative thereof which is capable of delivering about 1 g to 5 g or more of ketone bodies to a subject diagnosed with a glucose hypometabolic disorder. In certain embodiments, R¹ is a amino acid or a derivative thereof which is capable of delivering about 5 g to 10 g or more of ketone bodies to a subject diagnosed with a glucose hypometabolic disorder. In certain embodiments, R¹ is a amino acid or a derivative thereof which is capable of delivering about 10 g to 15 g or more of ketone bodies to a subject diagnosed with a glucose hypometabolic disorder. In certain embodiments, R¹ is a amino acid or a derivative thereof which is capable of delivering about 15 g to 20 g or more of ketone bodies to a subject diagnosed with a glucose hypometabolic disorder. In certain embodiments, R¹ is a amino acid or a derivative thereof which is capable of delivering about 20 g to 25 g or more of ketone bodies to a subject diagnosed with a glucose hypometabolic disorder. In certain embodiments, R¹ is a amino acid or a derivative thereof which is capable of delivering about 25 g to 30 g or more of ketone bodies to a subject diagnosed with a glucose hypometabolic disorder. In certain embodiments, R¹ is a amino acid or a derivative thereof which is capable of delivering about 30 g to about 35 g or more of ketone bodies to a subject diagnosed with a glucose hypometabolic disorder. In certain embodiments, R¹ is a amino acid or a derivative thereof which is capable of delivering about 35 g to 40 g or more of ketone bodies to a subject diagnosed with a glucose hypometabolic disorder. In certain embodiments, R¹ is a amino acid or a derivative thereof which is capable of delivering about 40 g to 45 g or more of ketone bodies to a subject diagnosed with a glucose hypometabolic disorder. In certain embodiments, R¹ is a amino acid or a derivative thereof which is capable of delivering or about 45 g to 50 g or more of ketone bodies to a subject diagnosed with a glucose hypometabolic disorder.

In certain embodiments, R¹ is a amino acid or a derivative thereof which is capable of delivering about 1 g or more of ketone bodies to a subject diagnosed with a glucose hypometabolic disorder. In certain embodiments, R¹ is a amino acid or a derivative thereof which is capable of delivering about 5 g or more of ketone bodies to a subject diagnosed with a glucose hypometabolic disorder. In certain embodiments, R¹ is a amino acid or a derivative thereof which is capable of delivering about 10 g or more of ketone bodies to a subject diagnosed with a glucose hypometabolic disorder. In certain embodiments, R¹ is a amino acid or a derivative thereof which is capable of delivering about 15 g or more of ketone bodies to a subject diagnosed with a glucose hypometabolic disorder. In certain embodiments, R¹ is an amino acid or a derivative thereof which is capable of delivering about 20 g or more of ketone bodies to a subject diagnosed with a glucose hypometabolic disorder. In certain embodiments, R¹ is a amino acid or a derivative thereof which is capable of delivering about 25 g or more of ketone bodies to a subject diagnosed with a glucose hypometabolic disorder. In certain embodiments, R¹ is an amino acid or a derivative thereof which is capable of delivering about 30 g or more of ketone bodies to a subject diagnosed with a glucose hypometabolic disorder. In certain embodiments, R¹ is a amino acid or a derivative thereof which is capable of delivering about 35 g or more of ketone bodies to a subject diagnosed with a glucose hypometabolic disorder. In certain embodiments, R¹ is a amino acid or a derivative thereof which is capable of delivering about 40 g or more of ketone bodies to a subject diagnosed with a glucose hypometabolic disorder. In certain embodiments, R¹ is a amino acid or a derivative thereof which is capable of delivering or about 45 g or more of ketone bodies to a subject diagnosed with a glucose hypometabolic disorder. In certain embodiments, R¹ is a amino acid or a derivative thereof which is capable of delivering or about 50 g or more of ketone bodies to a subject diagnosed with a glucose hypometabolic disorder.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine.

In certain embodiments, R¹ is selected from the group consisting of trehalose, inositol, ribose, inulin, creatine, glycine, carnitine, and taurine. In certain embodiments, R¹ is selected from the group consisting of trehalose, myo-inositol, D-ribose, inulin, creatine, glycine, carnitine, taurine, glycerol, allulose, aspartic acid, and carnosine. In certain embodiments, R¹ is selected from the group consisting of trehalose, myo-inositol, D-ribose, inulin, creatine, glycine, carnitine, and taurine.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

As generally defined above, X is a covalent linker joining selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—. In certain embodiments, X is —O—C(O)—. In certain embodiments, X is —C(O)—O—. In certain embodiments, X is —C(O)—NH—. In certain embodiments, X is —NH—C(O)—. In certain embodiments, X is —C(O)—S—. In certain embodiments, X is —S—C(O)—. In certain embodiments, X is —O—C(O)—O—. In certain embodiments, X is —O—C(O)—NH— or —NH—C(O)—O—. In certain embodiments, X is —O—P(O₂)—O—. In certain embodiments, X is —O—SO₂—O—. In certain embodiments, X is —C═N—. In certain embodiments, X is —C═N—NH—. In certain embodiments, X is —O—. In certain embodiments, X is —NH—. In certain embodiments, X is —S—. In certain embodiments, X is —S—S—. In preferred embodiments, X is —O— or —NH—.

In certain preferred embodiments, R¹ is trehalose, myo-inositol, or D-ribose. In certain embodiments, R¹ is myo-inositol or trehalose. In certain embodiments, R¹ is myo-inositol. In certain embodiments, R¹ is trehalose. In certain embodiments, R¹ is D-ribose. In certain embodiments, R¹ is inulin. In certain embodiments, R¹ is creatine. In certain embodiments, R¹ is glycine. In certain embodiments, R¹ is carnitine. In certain embodiments, R¹ is taurine. In certain embodiments, R¹ is glycerol. In certain embodiments, R¹ is allulose. In certain embodiments, R¹ is aspartic acid. In certain embodiments, R¹ is carnosine.

In certain embodiments, the pharmaceutical composition comprises one unique compound of Formula (A) is:

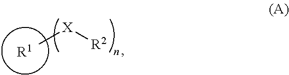

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from β-hydroxybutyryl or a derivative thereof, and
n is an integer from 1 to 50, wherein n represents the total number of ketone body moieties attached to R¹, and wherein n can be less than or equal to the total number of available attachment sites on R¹, and
wherein the other unique compound of Formula (A) is:

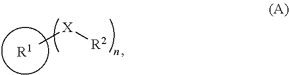

or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O—SO₂—O—, —C═N—, —C═N—NH—, —O—, —NH—, —S—, and —S—S—;
R² is a ketone body moiety selected from acetoacetyl (AcAc) or a derivative thereof; and
n is an integer from 1 to 50, wherein n represents the total number of ketone body moieties attached to R¹, and wherein n can be less than or equal to the total number of available attachment sites on R¹.

In some embodiments, the pharmaceutical compositions are administered in either single or multiple doses by any of the accepted modes of administration of agents having similar utilities, including rectal, buccal, intransal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. In some embodiments, the forms in which the novel compositions of the present disclosure are incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also used for injection. In some embodiments, ethanol, glycerol, propylene glycol, liquid polyethylene glycol, and the like (and suitable mixtures thereof), cyclodextrin derivatives, and vegetable oils are employed. In some embodiments, the proper fluidity is maintained, for example, by the use of a coating, such as lecithin, by the maintenance of the required particle size in the case of dispersion and by the use of surfactants. In some embodiments, the prevention of the action of microorganisms is brought about by various antibacterial and antifungal agents, for example, parabens, chlorobutanol, phenol, sorbic acid, thimerosal, and the like.

Sterile injectable solutions are prepared by incorporating a compound according to the present disclosure in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of the compounds disclosed herein. In some embodiments, administration is via capsule or enteric coated tablets, or the like. In some embodiments, in making the pharmaceutical compositions that include at least one compound described herein, the active ingredient is usually diluted by an excipient and/or enclosed within such a carrier that is in the form of a capsule, sachet, paper or other container. In some embodiments, when the excipient serves as a diluent, it is in the form of a solid, semi-solid, or liquid material (as above), which acts as a vehicle, carrier or medium for the active ingredient. Thus, in some embodiments, the compositions are e in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. In some embodiments, the formulations additionally include: lubricating agents such as talc, magnesium stearate, and mineral oil; wetting agents; emulsifying and suspending agents; preserving agents such as methyl and propylhydroxy-benzoates; sweetening agents; and flavoring agents.

In some embodiments, the compositions of the disclosure are formulated so as to provide quick, sustained or delayed release of the active ingredient after administration to the patient. Controlled release drug delivery systems for oral administration include osmotic pump systems and dissolutional systems containing polymer-coated reservoirs or drug-polymer matrix formulations. Another formulation for use in the methods of the present disclosure employs transdermal delivery devices (“patches”). In some embodiments, such transdermal patches are used to provide continuous or discontinuous infusion of the compounds of the present disclosure in controlled amounts. The construction and use of transdermal patches for the delivery of pharmaceutical agents is well known in the art. In some embodiments, such patches are constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 15 g (e.g., 10 g) of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. In some embodiments, when referring to these preformulation compositions as homogeneous, it is meant that the active ingredient is dispersed evenly throughout the composition so that the composition is readily subdivided into equally effective unit dosage forms such as tablets, pills and capsules.

In some embodiments, the tablets or pills of the present disclosure are coated or otherwise compounded to provide a dosage form affording the advantage of prolonged action, or to protect from the acid conditions of the stomach. For example, in some embodiments, the tablet or pill comprises an inner dosage and an outer dosage component, the latter being in the form of an envelope over the former. In some embodiments, the two components are separated by an enteric layer that serves to resist disintegration in the stomach and permit the inner component to pass intact into the duodenum or to be delayed in release. In some embodiments, a variety of materials are used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. In some embodiments, the liquid or solid compositions contain suitable pharmaceutically acceptable excipients as described supra. In some embodiments, the compositions are administered by the oral or nasal respiratory route for local or systemic effect. In some embodiments, compositions in pharmaceutically acceptable solvents are nebulized by use of inert gases. In some embodiments, nebulized solutions are inhaled directly from the nebulizing device or the nebulizing device is attached to a facemask tent, or intermittent positive pressure breathing machine. In some embodiments, solution, suspension, or powder compositions are administered, orally or nasally, from devices that deliver the formulation in an appropriate manner.

### EXAMPLES

The following examples are provided to illustrate the application of the disclosed compounds and compositions in practice. They are not intended to limit the scope of the invention but rather to demonstrate certain embodiments and outcomes.

### Abbreviations

AcAc Acetoacetate
ACN Acetonitrile
BHB Beta-hydroxybutyrate
DI H₂O Deionized water
DMAP 4-(Dimethylamino)pyridine
EDC N-Ethyl-N′-(3-dimethylaminopropyl)carbodiimide
K₂CO₃ Potassium carbonate
LiOH Lithium hydroxide monohydrate
MeAA methyl acetoacetate
MeOH Methanol
MgSO₄ Magnesium sulfate
MTBE tert-Butyl methyl ether
Na₂SO₄ Sodium sulfate
NaCl Sodium chloride
TBA tert-Butyl ammonium
TBAA tert-Butyl acetoacetate
TBAF tetrabutylammonium fluoride
TBAI tert-Butyl ammonium Iodide
TBDPS tert-Butyl diphenyl silane
TEA triethylamine
THF Tetrahydrofuran

### Methods of Synthesis

The compounds of the present invention may be synthesized by various methods known in the art. Generally, the synthesis involves the esterification or amidation of a carrier molecule containing multiple hydroxyl or amine groups with a ketone body or a protected form thereof.

For example, Trehalose-BHB Ester may be synthesized by:

Step 1: Protection of β-hydroxybutyric acid as a suitable ester (e.g., methyl or benzyl ester).

Step 2: Activation of the carboxylic acid group using coupling reagents such as DCC/DMAβ, HATU, or EDC/HOBt.

Step 3: Reaction with trehalose to form the ester linkages.

Step 4: Deprotection to yield the final product.

Similarly, amide conjugates such as Glycine-Acetoacetate Amide may be synthesized by activation of acetoacetic acid followed by coupling with the amino group of glycine.

Detailed synthetic procedures for specific compounds will be described in the Examples below.

### Example 1: Synthesis of Trehalose-BHB Ester (TrehaBHB)

### Synthesis of Trehalose 2,3,4,2′,3′,4′,6,6′—Octa-(3-hydroxybutyrate) ester

Step 1: Protection of the hydroxyl group of β-hydroxybutyric acid using tert-butyldimethylsilyl chloride (TBDMSCI) in the presence of imidazole and DMF.

Step 2: Activation of the carboxylic acid group using N,N′-dicyclohexylcarbodiimide (DCC) and 4-dimethylaminopyridine (DMAβ) in dichloromethane.

Step 3: Reaction with trehalose dissolved in a mixture of dichloromethane and dimethylformamide at room temperature for 48 hours.

Step 4: Deprotection of the TBDMS groups using tetrabutylammonium fluoride (TBAF) in THF.

Step 5: Purification by column chromatography to yield Trehalose-BHB Ester as a white solid.

### Example 2: Synthesis of Inositol-BHB Ester (InosBHB)

### Synthesis of Myo-Inositol-1,2,3,4,5,6-Hexa-(3-hydroxybutyrate) ester

Step 1: Protection of the hydroxyl group of β-hydroxybutyric acid using benzyl bromide and potassium carbonate in DMF.

Step 2: Conversion of the carboxylic acid to the acid chloride using oxalyl chloride in dichloromethane with catalytic DMF.

Step 3: Reaction with myo-inositol in pyridine and dichloromethane at 0° C., gradually warming to room temperature over 24 hours.

Step 4: Hydrogenolysis of the benzyl protecting groups using Pd/C catalyst under hydrogen atmosphere.

Step 5: Purification by recrystallization to yield Inositol-BHB Ester as a white crystalline solid.

### Example 3: Synthesis of Trehalose-Acetoacetate Ester (TrehaAce)

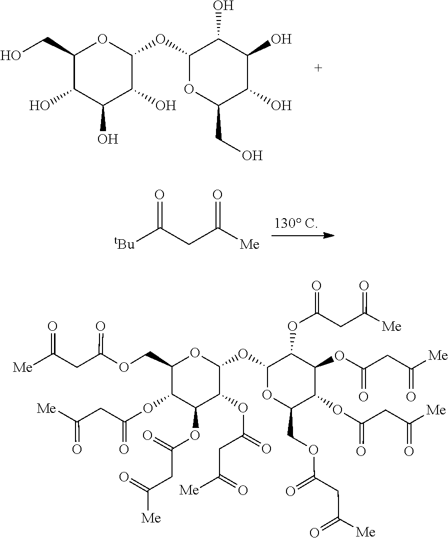 [SMILES: OC[C@H]1O[C@H](O[C@H]2O[C@H](CO)[C@@H](O)[C@H](O)[C@H]2O)[C@H](O)[C@@H](O)[C@@H]1O ; CC(=O)CC(=O)C(C)(C)C ; CC(=O)CC(=O)OC[C@H]1O[C@H](O[C@H]2O[C@H](COC(=O)CC(C)=O)[C@@H](OC(=O)CC(C)=O)[C@H](OC(=O)CC(C)=O)[C@H]2OC(=O)CC(C)=O)[C@H](OC(=O)CC(C)=O)[C@@H](OC(=O)CC(C)=O)[C@@H]1OC(=O)CC(C)=O]

Trehalose (10 g, 29.2 mmol) was charged into a jacketed reactor equipped with a Dean-Stark trap. TBAA (92.4 g, 580 mmol) was added and the mixture was stirred under nitrogen at 130° C. for 16 hrs. The reaction mixture was placed on rotary evaporator (<10 mbar, 80° C.) to remove excess TBAA. 28.3 g of oil recovered, 95.4%.

¹H NMR (CDCl₃, 400 MHz) δ 5.55 (m, 2H), 5.31 (m, 2H), 5.12-4.9 (m, 4H), 4.40-4.28 (m, 4H), 4.15-3.84 (m, 2H), 3.70-3.45 (m, 16H), 2.33-2.19 (m, 24H).

¹³C NMR (CDCl₃, 100 MHz) δ 199.65, 199.58, 199.42, 199.34, 165.73, 165.19, 165.11, 164.89, 90.33, 68.89, 68.49, 67.76, 67.00, 61.79, 48.98, 48.83, 48.63, 48.48, 29.40, 29.33, 29.22, 29.18.

(MS) (ES−) m/z 1013.96

### Example 4: Synthesis of Inositol-Acetoacetate Ester (InosAce)

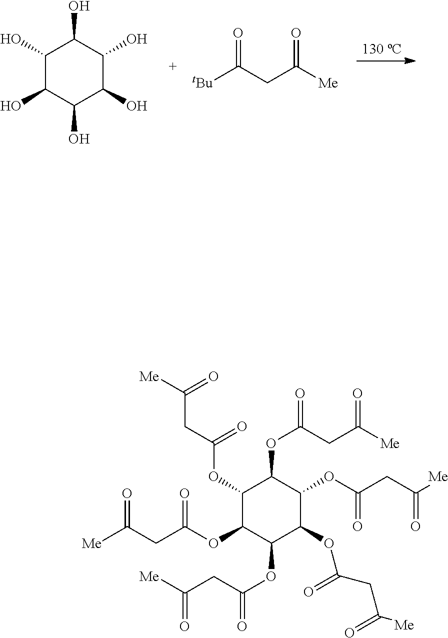 [SMILES: O[C@H]1[C@H](O)[C@@H](O)[C@H](O)[C@@H](O)[C@H]1O ; CC(=O)CC(=O)C(C)(C)C ; CC(=O)CC(=O)O[C@H]1[C@H](OC(=O)CC(C)=O)[C@@H](OC(=O)CC(C)=O)[C@H](OC(=O)CC(C)=O)[C@@H](OC(=O)CC(C)=O)[C@H]1OC(=O)CC(C)=O]

Myo-Inositol (10 g, 55.2 mmol) was charged into a jacketed reactor equipped with a Dean-Stark trap. TBAA 110 mL, 670 mmol) was added and the mixture was stirred under nitrogen at 130° C. for 16 hrs. The reaction mixture was placed on rotary evaporator (<10 mbar, 80° C.) to remove excess TBAA. 37.8 g of oil recovered, 99.54%.

¹H NMR (CDCl₃, 400 MHz) δ 5.17-4.92 (m, 6H), 3.65-3.49 (m, 12H), 2.31-2.18 (m, 18H).

¹³C NMR (CDCl₃, 100 MHz) δ 201.41, 200.60, 200.55, 200.18, 166.14, 166.02, 165.98, 165.88, 68.95, 68.76, 68.69, 68.64, 49.96, 49.86, 49.42, 49.35.

(MS) (ES−) m/z 683.88

### Example 5: Synthesis of Glycerol-Acetoacetate Ester (GlycAce)

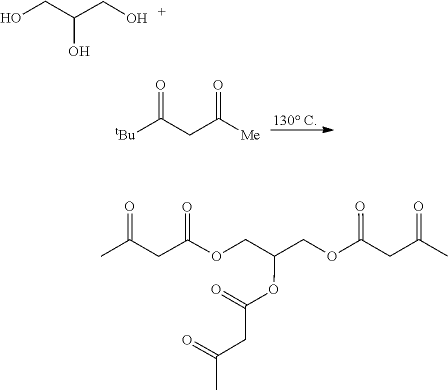 [SMILES: OCC(O)CO ; CC(=O)CC(=O)C(C)(C)C ; CC(=O)CC(=O)OCC(COC(=O)CC(C)=O)OC(=O)CC(C)=O]

Glycerol (10 g, 108.58 mmol) was charged into a jacketed reactor equipped with a Dean-Stark trap. MAA (70.4 mL, 657.3 mmol) was added and the mixture was stirred under nitrogen at 130° C. for 40 hrs. The reaction mixture was placed on rotary evaporator (<10 mbar, 80° C.) to remove excess MAA. 36.1 g amber oil recovered, 96.6%.

¹H NMR (CDCl₃, 400 MHz) δ 5.32 (m, 1H), 4.32-4.16 (m, 4H), 3.80-3.46 (m, 6H), 2.25 (m, 9H).

¹³C NMR (CDCl₃, 100 MHz) δ 201.25, 200.91, 166.89, 166.83, 69.55, 67.49, 65.55, 49.86, 49.61.

(MS) (ES+) m/z 345.95

### Example 6: Synthesis of AcAc-Allulose

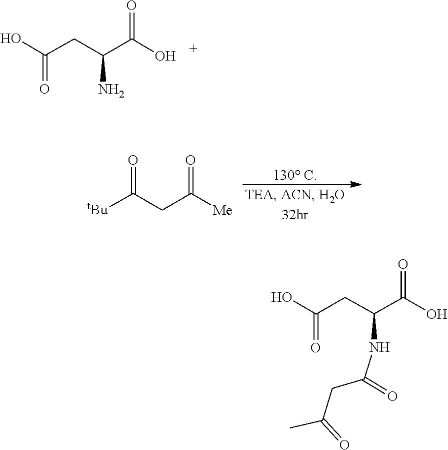

Allulose (25 g, 131.8 mmol) was charged into a jacketed reactor equipped with a Dean-Stark trap. TBAA (220 mL, 1300 mmol) was added and the mixture was stirred under nitrogen at 130° C. for 16 hrs. The reaction mixture was placed on rotary evaporator (<10 mbar, 80 C) to remove excess TBAA. 53.3 g amber oil recovered, 69.9%.

¹H NMR (CDCl₃, 400 MHz) δ 5.42-5.34 (m, 3H), 5.04 (m, 4H), 3.57-3.39 (m, 10H), 2.25-2.17 (m, 15H).

¹³C NMR (CDCl₃, 100 MHz) δ 207.04, 201.19, 200.08, 166.39, 82.00, 76.72, 51.53, 49.75, 49.48, 30.93, 30.21, 30.00, 27.96.

(MS) (ES−) m/z 599.53

### Example 7: Synthesis of Aspartic Acid-Acetoacetate Ester (AspAce)

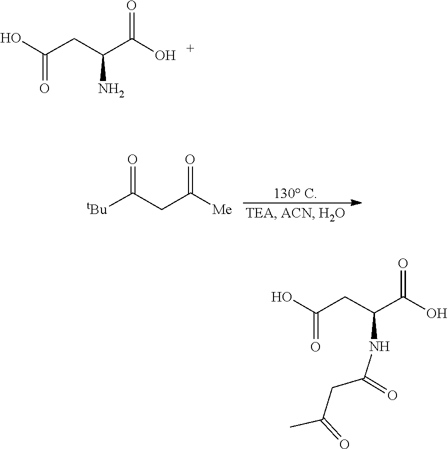

L-aspartic acid (20 g, 150.3 mmol) was charged into a jacketed reactor with acetonitrile (7 vols), H2) (2 Vols), and TEA (42 mL, 415.1 mmol) equipped with a Dean-Stark trap. TBAA (37 mL, 220 mmol) was added and the mixture was stirred under nitrogen at 130° C. for 48 hrs. The reaction mixture was placed on rotary evaporator (<10 mbar, 80° C.) to remove excess TBAA. 31.33 g recovered, 95.9%.

¹HNMR (CDCl₃, 400 MHz) δ 9.16 (m, 1H), 8.11 (m, 1H), 4.20 (m, 1H) 2.5 (m, 2H), 2.38 (m, 2H), 2.13 (s, 3H).

¹³C NMR (CDCl₃, 100 MHz) δ 150.06, 145.51, 128.16, 124.38, 49.21, 48.38, 37.46, 29.90.

(MS) (ES−) m/z 216.22

### Example 8: Synthesis of Taurine-Acetoacetate Amide (TaurAce)

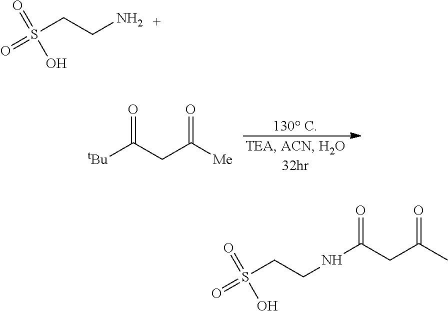

Taurine (25 g, 199.8 mmol) was charged into a jacketed reactor with acetonitrile (175 mL, 7 vols), H2O (75 mL, 2 Vols), and TEA (56 mL 402.mmol) equipped with a Dean-Stark trap. TBAA (37 mL, 220 mmol) was added and the mixture was stirred under nitrogen at 130° C. for 48 hrs. The reaction mixture was placed on rotary evaporator (<10 mbar, 80° C.) to remove excess TBAA. 40.1 g recovered, 95.9%.

¹HMNR (CDCl₃, 400 MHz) δ 7.61 (bs, 1H) 7.28 (s, 1H), 3.63 (dt, 2H), 3.3 (m, 2H), 2.92 (dt, 2H), 2.18 (s, 3H).

¹³C NMR (CDCl₃, 100 MHz) δ 207.83, 169.03, 49.53, 46.67, 35.21, 29.70.

(MS) (ES−) m/z 208.16

### Example 9: Synthesis of Glycerol-BHB Ester (GlycBHB)

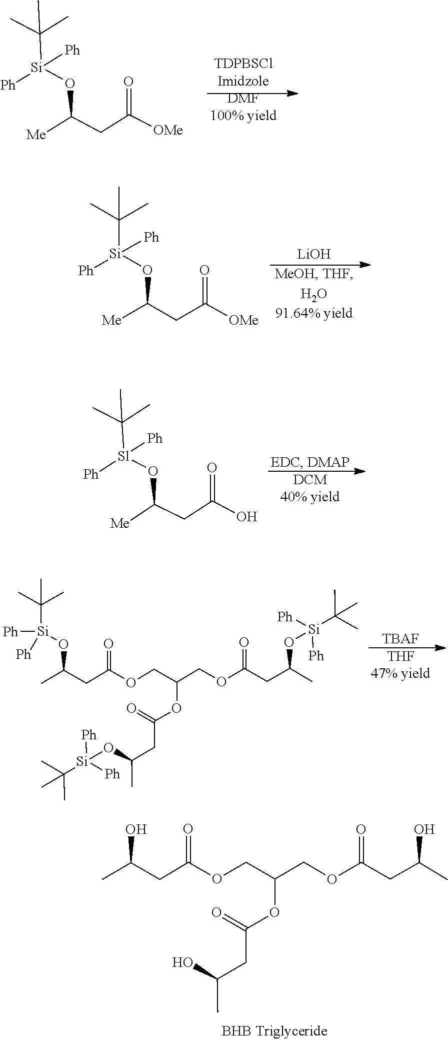

Step 1: BHB was treated with TBDPSCl in the presence of imidazole and DMF to yield TBDPS protected BHB in quantitative yield. The crude product was hydrolyzed with LiOH to afford the TBDPS-BHB acid. The acid was coupled with glycerol using EDC to give the TrisTBDPS BHB Triglyceride. Deprotection with TBAF afforded BHB-Triglyceride. The final compound contained TBA as an impurity which hampered full characterization.

Step 2: Methyl-(R)-3-hydroxybutrate (28 g, 237 mmol) was stirred DMF (420 mL) at 0° C. Imidazole (24.2 g, 237 mmol) and TBDPSCl (74 mL, 284 mmol) were added and allowed to warm to RT while stirring overnight. The reaction was dilute with MTBE (280 mL) and aqueous layer removed. The organic layer was washed with brine (423 mL) and dried over Na₂SO₄. The solution was concentrated by rotavap and continued forward without purification.

Step 3: Methyl (3R)-3-[tert-butyl(diphenyl)silyl]oxybutanoate (84.5 g, 237 mmol) was stirred in MeOH (147 mL) and THF (322 mL) at RT. LiOH (14.5 g, 593 mmol) in H₂O (177 mL) was added and stirred at RT overnight. The reaction was diluted with MTBE (423 mL) and washed with H₂O (280 mL). The organic layer was washed with brine (280 mL) and dried over Na₂SO₄. The solution was concentrated by rotavap and continued further without purification.

Step 4: Glycerol (4 g, 43 mmol) was added to (3R)-3-[tert-butyl(diphenyl)silyl]oxybutanoic acid (44.6 g, 130 mmol) in DCM (200 mL) and stirred. DMAP (32 g, 260 mmol) and EDC (50.0 g, 261 mmol) were added and stirred overnight. The reaction mixture was washed with 1M HCl (100 mL), saturated NaHCO₃ (100 mL), and dried over Na₂SO₄. The reaction mixture was concentrated and the product purified by column chromatography to yield 16.6 g of TrisTBDPS-BHB Triglyceride.

Step 5: TrisTBDPS BHB Triglyceride (16.6 g) was stirred in THF (83 mL) at RT. 1M TBAF solution (140 mL) was added and stirred overnight. The reaction mixture was concentrated by rotavap and purified by reverse phase column chromatography to yield 2.46 g of β-BHB Triglyceride.

¹HNMR (CDCl₃, 400 MHz) δ 3.95 (m, 1H), 3.29 (m, 6H), 2.30 (m, 2H), 2.07 (m, 2H), 0.99 (m, 15).

### Example 10: Synthesis of Carnosine-BHB Ester (CarnosBHB)

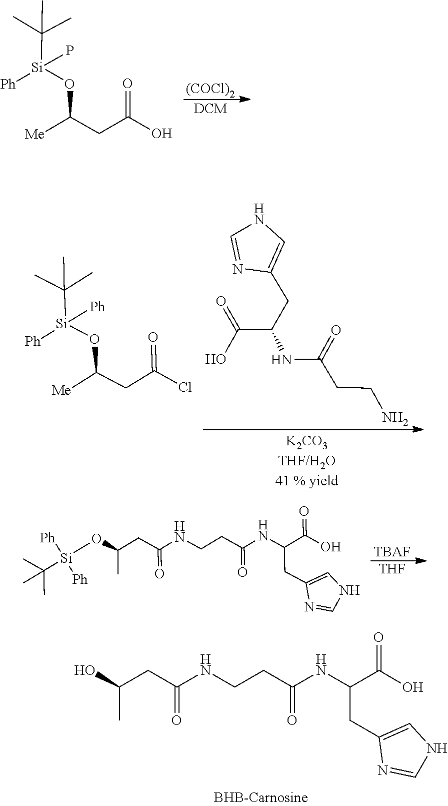

Step 1: TBDPS protected BHB was reacted with oxayl chloride to afford TBDPS protected BHB acid chloride. The acid chloride was reacted with carnosine in the presence of K₂CO₃ in THF/H2O to afford the coupled product in 41% yield. Deprotection with TBAF affords β-BHB carnosine. Final compound contained TBA impurity that hampered full characterization of this compound.

Step 2: (3R)-3-[tert-butyl(diphenyl)silyl]oxybutanoic acid (48 g, 140 mmol) was stirred in DCM (96 mL) at 0° C. Oxalyl chloride (14.6 mL, 168 mmol) was added. DMF (25 drops) was added and stirred for 30 min. The reaction was allowed to warm to RT while stirring for 2 hr. The mixture was concentrated by rotavap and used without further purification.

Step 3: (3R)-3-[tert-butyl(diphenyl)silyl]oxybutanoyl chloride was stirred in THF. Carnosine (6.4 g, 28.3 mmol) was added. K₂CO₃ (19.5 g, 141 mmol) in H2O (85 mL) was added. The reaction was stirred at RT over 48 hr. The reaction mixture concentrated by rotavap to remove THF. The aqueous mixture was extracted with EtOAc (150 mL), and dried over Na₂SO₄, and concentrated. The product was purified by reverse phase flash chromatography to yield.

Step 4: TBDPS β-HBH carnosine (7.32 g 23 mmol) was stirred in THF (65 mL) at RT. 1M TBAF solution (70 mL) was added and stirred overnight. The reaction mixture was concentrated by rotavap. Solids were filtered and the filtrate extracted with DCM (100 mL). The aqueous layer was concentrated by rotavap and purified by reverse phase column chromatography to yield 4.5 g of β-BHB-carnosine.

(MS) (ES−) m/z 311.11

### Biological Assays

### Example 11: Exemplary Effects of Ketone Bodies in Alzheimer's Disease Neurons

Described below are the effects of exogenous ketone bodies, including acetoacetate (AcAc), on neuronal viability, oxidative stress, mitochondrial function, and cellular energy metabolism in neurons derived from Alzheimer's disease (AD) patients. The results demonstrate that ketone treatment produces dose-dependent improvements across multiple functional endpoints relevant to impaired mitochondrial function and neurodegeneration.

### Dose-Dependent Improvement in Neuronal Viability

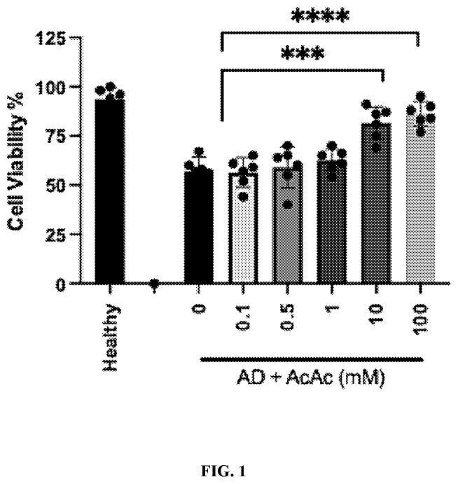

Exposure of AD-derived neurons to exogenous amyloid beta (Aβ1-42) resulted in a reduction in cell viability relative to healthy control neurons. Treatment with AcAc restored viability in a dose-dependent manner. As shown in FIG. 1, increasing concentrations of AcAc progressively improved cell viability in AD neurons exposed to Aβ, with higher concentrations approaching viability levels observed in healthy control cells. These results demonstrate that ketone treatment rescues neuronal survival under disease-relevant stress conditions.

### Reduction of Oxidative Stress

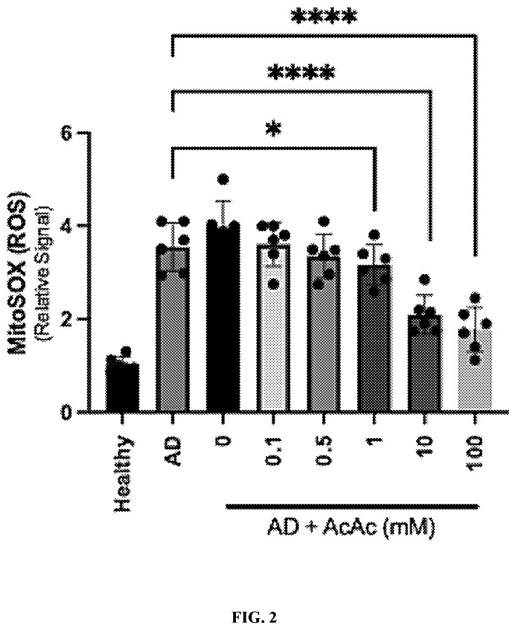

AD neurons exposed to amyloid beta exhibited elevated levels of reactive oxygen species (ROS) compared to healthy neurons, consistent with oxidative stress associated with neurodegeneration. Treatment with AcAc significantly reduced ROS levels in a dose-dependent manner. ROS levels were assessed using MitoSOX staining, which detects mitochondrial superoxide production, while mitochondrial structure and integrity were evaluated using MitoTracker staining. As shown in FIG. 2, AcAc treatment decreased MitoSOX relative signal intensity across increasing concentrations, indicating reduced oxidative stress.

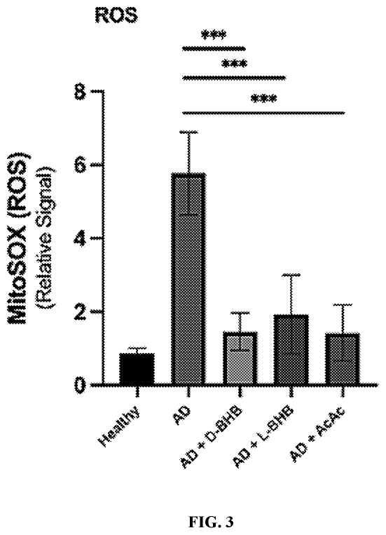

Consistent reductions in ROS were also observed in amyloid-induced oxidative stress model systems, following treatment with multiple ketone bodies, including D-β-hydroxybutyrate (D-BHB), L-β-hydroxybutyrate (L-BHB), and acetoacetate (AcAc), under non-dose-response conditions, as shown in FIG. 3, further supporting a class-wide effect of ketones on oxidative stress reduction.

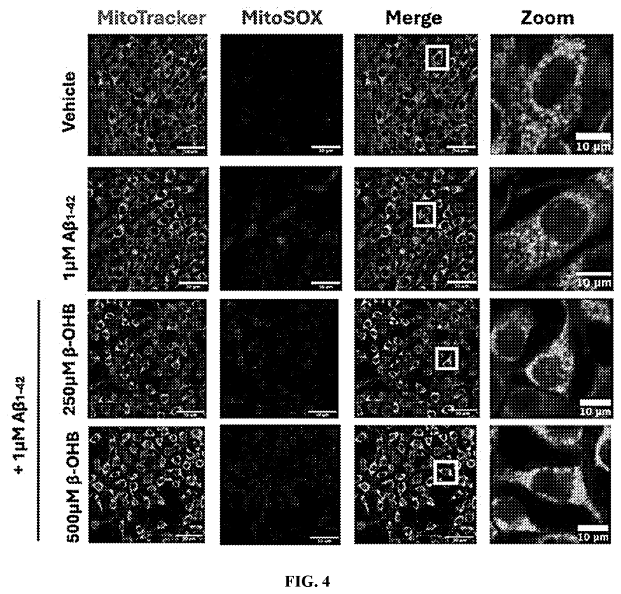

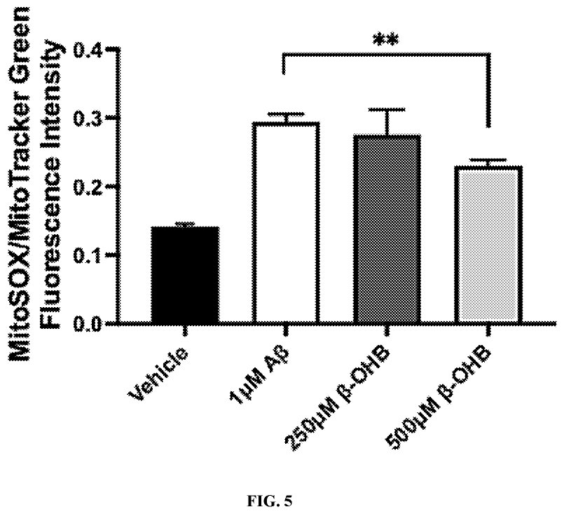

Consistent findings were also observed in amyloid-induced oxidative stress model systems, as shown in FIG. 4 and FIG. 5, where treatment with ketone bodies, including acetoacetate (AcAc) and β-hydroxybutyrate (β-OHB), reduced mitochondrial ROS levels and improved mitochondrial integrity, further supporting the role of ketones in mitigating oxidative damage in neuronal cells.

### Increase in ATP Production and Cellular Energy Metabolism

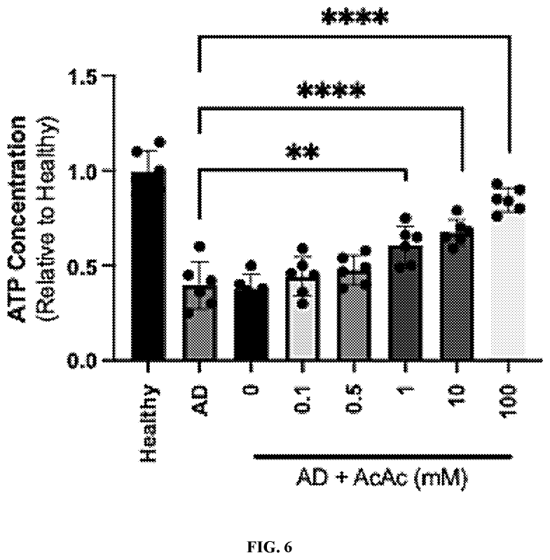

AD neurons demonstrated reduced intracellular ATP levels relative to healthy controls, consistent with impaired glucose utilization and mitochondrial dysfunction. Treatment with AcAc resulted in a dose-dependent increase in ATP concentration. As shown in FIG. 6, ATP levels increased with increasing AcAc concentration, indicating restoration of cellular energy production. These results are consistent with the ability of ketone bodies to serve as an alternative metabolic substrate and compensate for deficits in glucose metabolism.

### Reduction in Cell Death and Restoration of Neuronal Survival

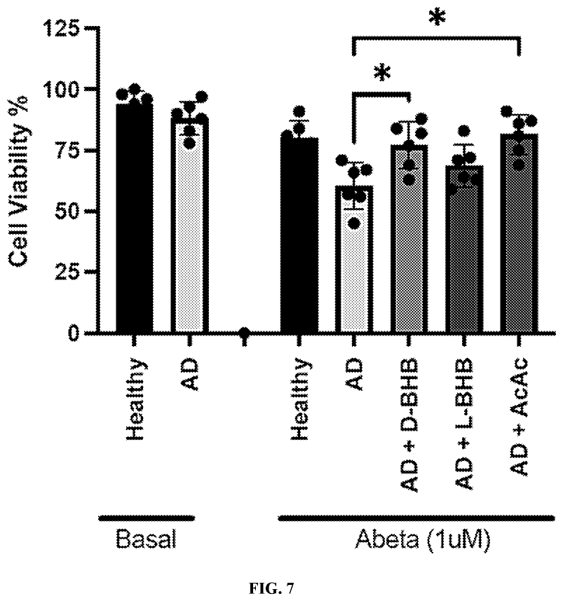

Exposure of AD neurons to amyloid beta resulted in a loss of cell viability and increased cell death relative to basal conditions. As shown in FIG. 7, no substantial differences in basal viability were observed between healthy and AD neurons; however, stimulation with Aβ resulted in reduced viability in AD neurons. Treatment with ketone bodies restored viability to varying extents, with D-β-hydroxybutyrate (D-BHB), L-β-hydroxybutyrate (L-BHB), and acetoacetate (AcAc) each improving viability relative to untreated AD neurons exposed to Aβ, and D-BHB and AcAc demonstrating substantial recovery toward levels observed in healthy control cells.

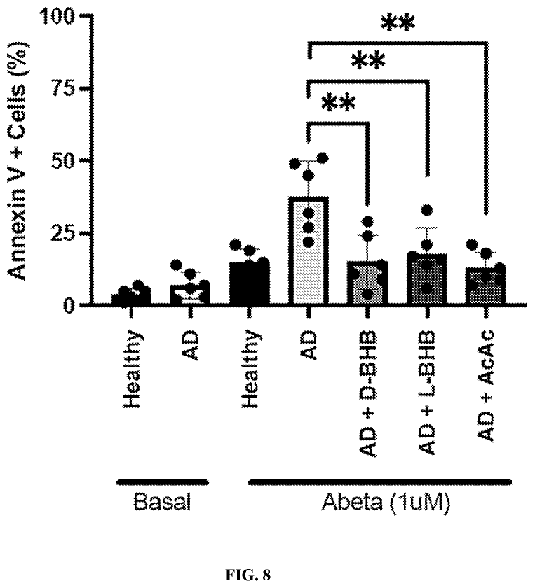

In addition to reductions in overall viability, Aβ exposure increased apoptotic signaling in AD neurons. As shown in FIG. 8, the percentage of Annexin V-positive cells was increased following Aβ treatment, indicating elevated apoptosis. Treatment with D-BHB, L-BHB, and AcAc reduced the proportion of Annexin V-positive cells relative to untreated AD neurons exposed to Aβ. These data indicate that ketone treatment protects neurons from amyloid-induced cell death and promotes neuronal survival.

### Improvement in Mitochondrial Function

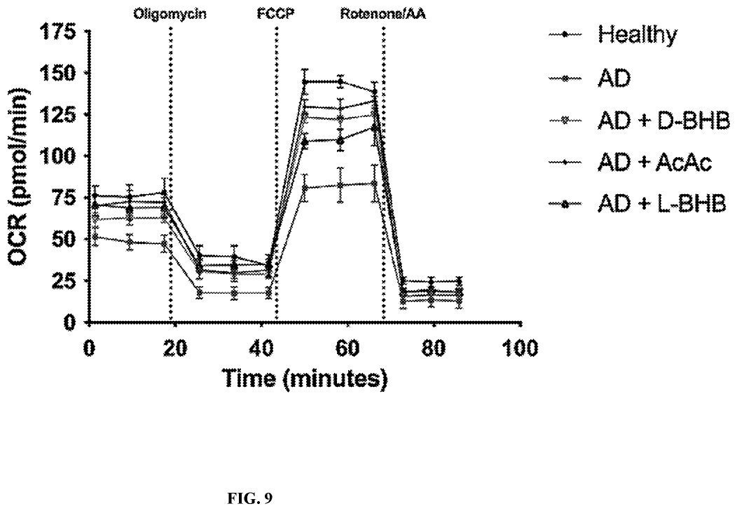

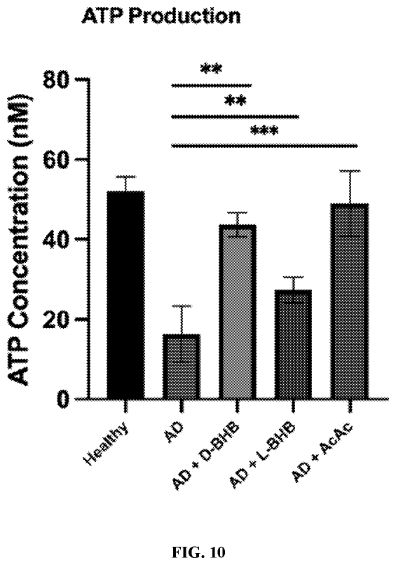

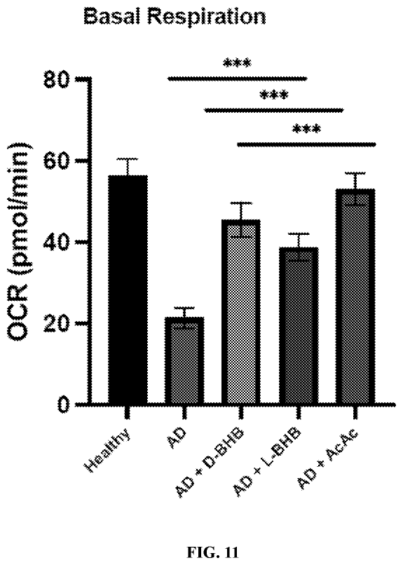

Mitochondrial dysfunction is a hallmark of AD and contributes to impaired energy metabolism. Treatment with ketones improved mitochondrial respiration and bioenergetic capacity in AD neurons. As shown in FIG. 9, ketone-treated AD neurons exhibited increased oxygen consumption rates compared to untreated AD neurons. Quantitative analysis further demonstrated improvements in ATP production and basal respiratory capacity, as shown in FIG. 10 and FIG. 11, respectively. These results indicate that ketone treatment restores mitochondrial function in AD neurons.

### Conclusion

Collectively, these data demonstrate that ketone bodies, including acetoacetate (AcAc) and others, provide a robust and dose-dependent rescue of neuronal dysfunction in Alzheimer's disease (AD) models. Ketone treatment improves neuronal viability, reduces oxidative stress, increases ATP production, decreases apoptosis, and enhances mitochondrial respiration. These findings support the use of exogenous ketone therapy to compensate for glucose hypometabolism and associated cellular energy deficits in neurodegenerative disease, consistent with the therapeutic titratable ketonemia (TTK) platform described herein.

### Methods

Human induced pluripotent stem cell (iPSC)-derived neurons were generated from both healthy donors and Alzheimer's disease (AD) patient donors and differentiated according to standard neuronal differentiation protocols. Cells were cultured under conditions sufficient to promote maturation prior to treatment.

For neuron dose-response experiments, neurons were exposed to varying concentrations of a ketone ester. Cells were treated for approximately 24 hours. To model disease-relevant stress conditions, neurons were co-treated with amyloid beta peptide (Aβ1-42) at a concentration of approximately 1 μM.

Cell viability was assessed using a luminescence-based live/dead assay (CellTiter-Glo, Promega). Reactive oxygen species (ROS) levels were measured using MitoSOX staining, which detects mitochondrial superoxide production. Mitochondrial structure and integrity were evaluated using MitoTracker staining. Intracellular ATP levels were quantified using an ATP-dependent luminescence assay (ATP-Glo, Promega).

For longer-term functional assays, including apoptosis and mitochondrial analyses, cells were treated for approximately 14 days prior to endpoint measurement. Apoptosis was measured using Annexin V staining to determine the percentage of apoptotic cells. Mitochondrial function was evaluated using a Seahorse XF Mito Stress Test (Agilent) to measure oxygen consumption rate (OCR), including basal respiration and ATP-linked respiration.

All assays were performed using multiple donor-derived cell lines and in replicate, and data were analyzed using standard statistical methods.

### Example 12: Exemplary Effects of Ketone Bodies in Parkinson's Disease Neurons

Described below are the effects of exogenous ketone bodies, including acetoacetate (AcAc) or compositions comprising one or more of the compounds described herein, on neuronal viability, oxidative stress, mitochondrial function, and cellular energy metabolism in neurons derived from Parkinson's disease (PD) patients. The results demonstrate that ketone treatment produces improvements across multiple functional endpoints relevant to impaired mitochondrial function and neurodegeneration.

### Reduction in Cell Death and Restoration of Neuronal Survival

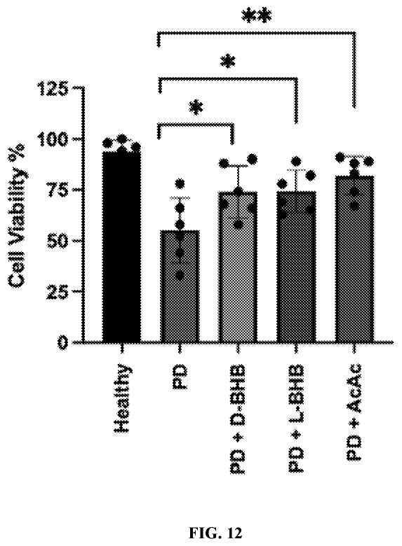

PD neurons exhibited a loss of cell viability and increased cell death relative to healthy control neurons. As shown in FIG. 12, PD neurons demonstrated reduced viability compared to healthy neurons, and treatment with ketone bodies, including D-β-hydroxybutyrate (D-BHB), L-β-hydroxybutyrate (L-BHB), and acetoacetate (AcAc), increased viability relative to untreated PD neurons. Among the tested ketone bodies, AcAc demonstrated a particularly robust effect in restoring viability toward levels observed in healthy control cells. These results demonstrate that ketone treatment improves neuronal survival in PD neurons.

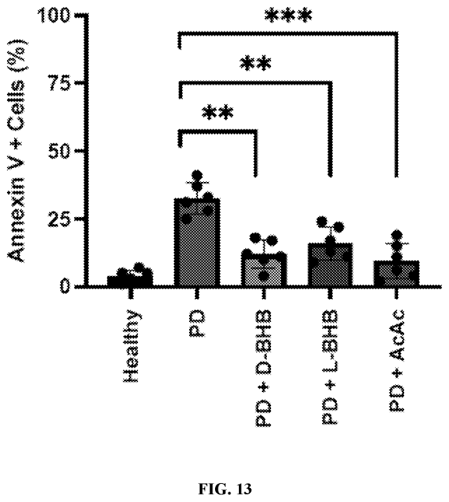

In addition to reductions in overall viability, PD neurons exhibited increased apoptotic signaling. As shown in FIG. 13, the percentage of Annexin V-positive cells was increased in PD neurons relative to healthy controls. Treatment with D-β-hydroxybutyrate (D-BHB), L-O— hydroxybutyrate (L-BHB), and acetoacetate (AcAc) reduced the proportion of Annexin V-positive cells relative to untreated PD neurons. These data indicate that ketone treatment protects neurons from disease-associated cell death and promotes neuronal survival.

### Reduction of Oxidative Stress

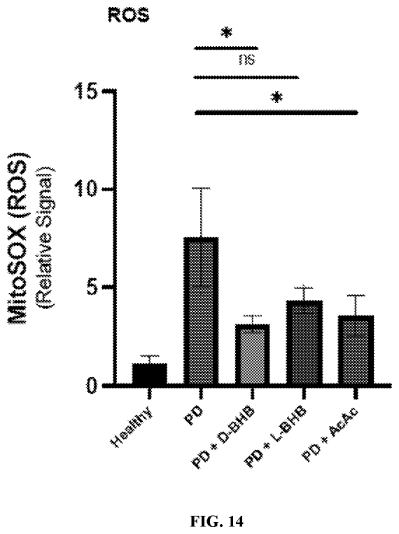

PD neurons exhibited elevated levels of reactive oxygen species (ROS) compared to healthy neurons, consistent with oxidative stress associated with neurodegeneration. Treatment with ketone bodies reduced ROS levels. ROS levels were assessed using MitoSOX staining, which detects mitochondrial superoxide production, while mitochondrial structure and integrity were evaluated using MitoTracker staining. As shown in FIG. 14, ketone treatment reduced MitoSOX relative signal intensity, indicating reduced oxidative stress. These findings further support the role of ketones in mitigating oxidative damage in neuronal cells.

### Improvement in Mitochondrial Function

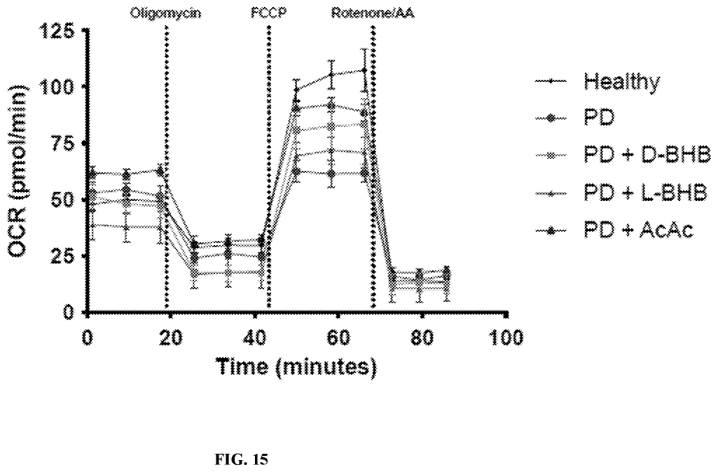

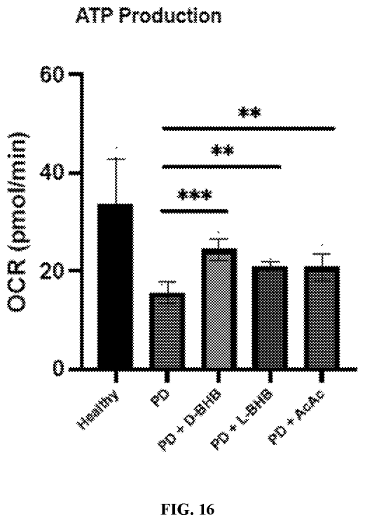

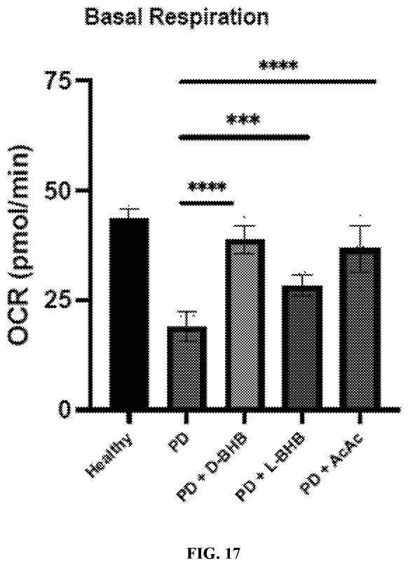

Mitochondrial dysfunction is a hallmark of PD and contributes to impaired energy metabolism. Treatment with ketones improved mitochondrial respiration and bioenergetic capacity in PD neurons. As shown in FIG. 15, ketone-treated PD neurons exhibited increased oxygen consumption rates compared to untreated PD neurons. Quantitative analysis further demonstrated improvements in ATP production and basal respiratory capacity, as shown in FIG. 16 and FIG. 17, respectively. These results indicate that ketone treatment restores mitochondrial function in PD neurons.

### Conclusion

Collectively, these data demonstrate that ketone bodies, including acetoacetate (AcAc) and others, provide a robust rescue of neuronal dysfunction in Parkinson's disease (PD) models.

Ketone treatment improves neuronal viability, reduces oxidative stress, increases ATP production, decreases apoptosis, and enhances mitochondrial respiration. These findings support the use of exogenous ketone therapy to address impaired cellular energy metabolism in neurodegenerative disease, consistent with the therapeutic titratable ketonemia (TTK) platform described herein.

### Methods

Human induced pluripotent stem cell (iPSC)-derived neurons were generated from both healthy donors and Parkinson's disease (PD) patient donors and differentiated according to standard neuronal differentiation protocols. Cells were cultured under conditions sufficient to promote maturation prior to treatment.

Cell viability was assessed using a luminescence-based live/dead assay (CellTiter-Glo, Promega). Reactive oxygen species (ROS) levels were measured using MitoSOX staining, which detects mitochondrial superoxide production. Mitochondrial structure and integrity were evaluated using MitoTracker staining. Intracellular ATP levels were quantified using an ATP-dependent luminescence assay (ATP-Glo, Promega). For functional assays, including apoptosis and mitochondrial analyses, cells were treated for approximately 14 days prior to endpoint measurement. Apoptosis was measured using Annexin V staining to determine the percentage of apoptotic cells. Mitochondrial function was evaluated using a Seahorse XF Mito Stress Test (Agilent) to measure oxygen consumption rate (OCR), including basal respiration and ATP-linked respiration.

All assays were performed using multiple donor-derived cell lines and in replicate, and data were analyzed using standard statistical methods.

### Example 13: Exemplary Effects of Ketone Bodies on Microglial Amyloid Uptake and Clearance

Described below are the effects of exogenous ketone bodies, including acetoacetate (AcAc) or compositions comprising one or more of the compounds described herein, on microglial uptake and clearance of amyloid beta (Aβ). The results demonstrate that ketone treatment enhances microglial phagocytic and degradative capacity, supporting a role for ketone bodies in promoting amyloid clearance in neurodegenerative disease.

### Enhancement of Amyloid Beta Uptake

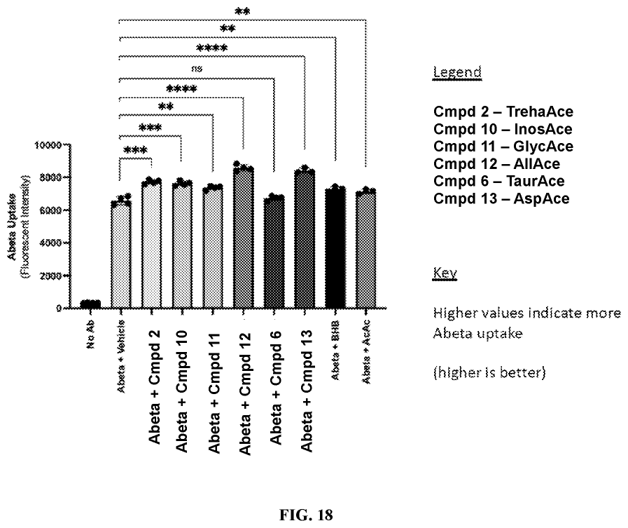

Microglia play a critical role in the internalization and clearance of extracellular amyloid beta. The ability of ketone bodies to enhance microglial uptake of Aβ was evaluated. As shown in FIG. 18, treatment with ketone bodies, including acetoacetate-derived ketone esters, increased the uptake of Aβ1-42 by microglia relative to untreated control cells. Increased intracellular fluorescence signal indicates enhanced internalization of amyloid beta. These results demonstrate that ketone treatment promotes microglial uptake of neurotoxic amyloid species.

### Enhancement of Amyloid Beta Clearance

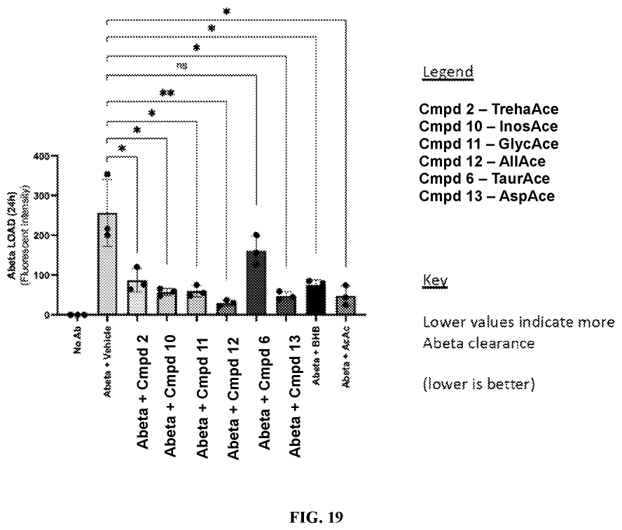

In addition to uptake, the ability of microglia to degrade and clear internalized amyloid beta is critical for maintaining neuronal health. As such, the effects of ketone treatment on amyloid clearance were evaluated. As shown in FIG. 19, treatment with ketone bodies reduced the amount of remaining extracellular amyloid beta following incubation, indicating enhanced clearance. Lower fluorescence signal corresponds to increased degradation and removal of amyloid beta. These data indicate that ketone treatment improves microglial clearance of amyloid beta.

### Acceleration of Amyloid Clearance Kinetics

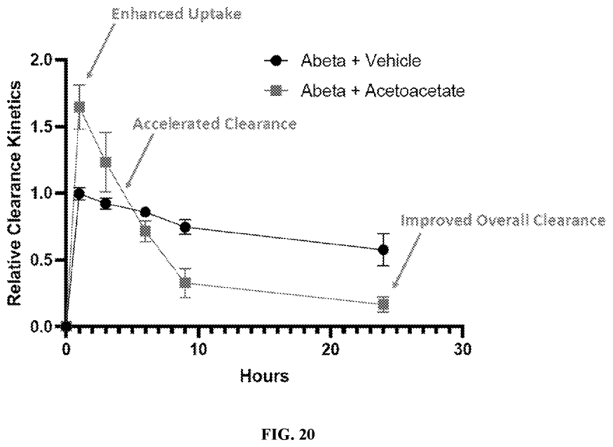

The rate of amyloid beta clearance was further evaluated to determine whether ketone bodies enhance the kinetics of amyloid degradation. As shown in FIG. 20, treatment with acetoacetate increased the rate of amyloid beta clearance over time relative to vehicle-treated controls. Microglia treated with ketone bodies exhibited both enhanced uptake and accelerated degradation of amyloid beta, resulting in improved overall clearance kinetics.

### Conclusion

Collectively, these data demonstrate that ketone bodies, including acetoacetate (AcAc), enhance both the uptake and clearance of amyloid beta by microglia. Ketone treatment increases internalization of amyloid, accelerates its degradation, and improves overall clearance kinetics.

These findings support a dual mechanism of action for ketone therapy in neurodegenerative disease, including both metabolic support and enhancement of microglial-mediated amyloid clearance and phagocytic capacity.

### Methods

Human induced pluripotent stem cell (iPSC)-derived microglia were generated and cultured under conditions sufficient to promote maturation prior to treatment. Microglial identity was confirmed by expression of established markers (i.e. Iba1).

For uptake assays, microglia were treated with ketone esters at a concentration of approximately 5 mM. Cells were exposed to fluorescently labeled amyloid beta peptide (Aβ1-42) and incubated for approximately 1 hour. Following incubation, cells were subjected to an acid wash to remove non-internalized amyloid, and intracellular fluorescence was measured to quantify amyloid uptake.

For clearance assays, microglia were treated with ketone esters and exposed to fluorescently labeled amyloid beta peptide. Cells were incubated for approximately 24 hours, and the amount of remaining amyloid beta was quantified by fluorescence-based analysis.

Amyloid uptake and clearance were quantified using fluorescence plate reader measurements. Data were analyzed by comparing treated and untreated conditions to determine relative changes in amyloid internalization and degradation.

All assays were performed in replicate, and data were analyzed using standard statistical methods.

### Example 14: PK Analysis of Representative Compound of Formula (A)

### Experimental Parameters

Compound: Representative Compound of Formula A, TrehaAce (Compound 2)
Study type 24 h PK, Beta-hydroxybutyrate levels as surrogate
Test animal: Strain/Species/Sex C57BL6J/Mouse/Male
Total number of animals used 6 (n=3/group)
Animal age/Body weight 8 weeks/23-26 g
Route of administration PO
Feeding regimen Fed—Ad libitum
Dose level (g/kg) Group 1—1 gpk, Group 2—5 gpk
Dose volume (mL/kg body weight) Up to 0.3 mL oral gavage
Formulation strength (mg/mL) 500 mg/mL
Vehicle 20% DMSO/80% water
Sampling technique Submandibular
Blood collection schedule, post dose 0, 0.08, 0.25, 0.5, 1.0, 2.0, 4.0, 8.0 and 24 h (8 time points)
Anticoagulant K2EDTA
Matrix Plasma
Study samples storage At −80° C.

### SUMMARY

TrehaAce was well tolerated at all study doses, and no indication of toxicity was observed. TrehaAce is a ketone ester prodrug that is rapidly converted to the functional ketone body betahydroxybutyrate (BHB) following oral consumption. In the present study, plasma BHB levels were utilized as a surrogate pharmacokinetic marker for the test article as reported previously. Baseline beta-hydroxybutyrate levels were assayed at the 0 timepoint.

Noncompartmental pharmacokinetic analysis of plasma BHB was performed following oral administration of TrehaAce at 1 g/kg and 5 g/kg in mice using standard Phoenix® WinNonlin extravascular NCA methods (linear trapezoidal AUC, log-linear terminal phase).

At both dose levels, TrehaAce was rapidly absorbed with a consistent Tmax of approximately 2 hours, indicating similar absorption kinetics across doses. Peak plasma concentrations (Cmax) increased with dose in an approximately dose-proportional manner.

However, systemic exposure (AUC) increased in a less-than-dose-proportional fashion from 1 g/kg to 5 g/kg, as evidenced by reduced dose-normalized AUC values at the higher dose. This finding suggests potential nonlinear pharmacokinetics, possibly due to rapid prodrug conversion, saturation of absorption, reduced oral bioavailability as dose increases, or dose-dependent clearance mechanisms.

At the 1 g/kg dose, terminal phase characterization was acceptable for two of three animals, although high AUC and AUMC extrapolation percentages indicate that later sampling time points would be beneficial for more robust estimation of terminal half-life and MRT. In contrast, the 5 g/kg dose group demonstrated a well-defined terminal phase in all animals, with lower extrapolated exposure and more stable Xz-dependent parameters.

Apparent clearance (CL/F) increased with dose, and apparent volume of distribution (Vz/F) decreased at the higher dose, further supporting the presence of nonlinear exposure kinetics.

Administration of TrehaAce at 1 gpkg and 5 gpkg resulted in a maximal induction of plasma BHB levels peaking at 2.0 h post-administration and achieving a mean Cmax of ~324 uM and ~1541 uM for each dose group respectively. Over the duration of the study a cumulative exposure (AUC0-t) reached approximately 2.3 mM and 11.8 mM for the 1 gpk and 5 gpk doses respectively.

The ~91% dose linearity was observed between 1 gpk and 5 gpk dosing and sustainable BHB levels above 0.5 mM at the 5 gpk dose over 8 h indicate single daily administration of the test article to induce a sustained ketogenic state without required fasting.

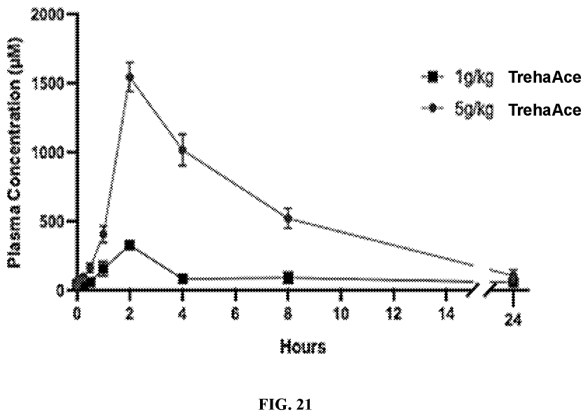

Results are summarized in FIG. 21 and in Table 3 below.

TABLE 3

| Parameter | 1g pk Dose | 5g pk Dose |
|---|---|---|
| Cₘₐₓ (uM) | 324.31 | 1541.67 |
| Tₘₐₓ (h) | 2.0 | 2.00 |
| Tₗₐₛₜ (h) | 24.0 | 24.0 |
| AUC₀₋ₜ (μM · h) | 2298.25 | 11762.82 |
| AUC_{0-∞} (μM . h) | 4045.43 | 12756.41 |
| T_{1/2} (h) | 20.46 | 6.33 |
| CL (mL/min/kg) | 39.63 | 63.2 |
| Vz/F (L/kg) | 70.29 | 34.47 |

1. A compound of Formula (A): 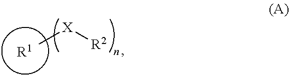
or a pharmaceutically acceptable salt or solvate thereof, wherein
R¹ is a polyol, amino acid, or a derivative thereof;
X is a covalent linker selected from the group consisting of —O—C(O)—, —C(O)—O—, —C(O)—NH—, —NH—C(O)—, —C(O)—S—, —S—C(O)—, —O—C(O)—O—, —O—C(O)—NH— or —NH—C(O)—O—, —O—P(O₂)—O—, —O−SO₂—O—, —C═N—, —C═N—NH—, —O—, —NH—, —S—, and —S—S—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

2. The compound of claim 1, wherein R¹ is selected from the group consisting of trehalose, myo-inositol, D-ribose, inulin, creatine, glycine, carnitine, taurine, glycerol, allulose, aspartic acid, and carnosine.

3. The compound of claim 1, wherein R¹ is trehalose, myo-inositol, or D-ribose.

4. The compound of claim 1, wherein X is —O—.

5. The compound of claim 1, wherein X is —NH—.

6. The compound of claim 1, wherein R² is β-hydroxybutyryl.

7. The compound of claim 1, wherein R² is acetoacetyl.

8. The compound of claim 1, wherein n is an integer from 1 to 20.

9. The compound of claim 1, wherein n is equal to the number of hydroxy groups in R¹.

10. The compound of claim 1, wherein the compound is compound of Formula (A): 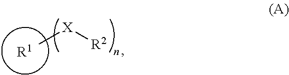
or a pharmaceutically acceptable salt or solvate thereof, wherein
R¹ is selected from the group consisting of trehalose, myo-inositol, D-ribose, inulin, creatine, glycine, carnitine, and taurine;
X is —O— or —NH—;
R² is a ketone body moiety selected from β-hydroxybutyryl, acetoacetyl, or a derivative thereof; and
n is an integer from 1 to 50, wherein n represents the total number of ketone body moieties attached to R¹, and wherein n can be less than or equal to the total number of available attachment sites on R¹.

11. The compound of claim 1, wherein the compound is selected from the group consisting of

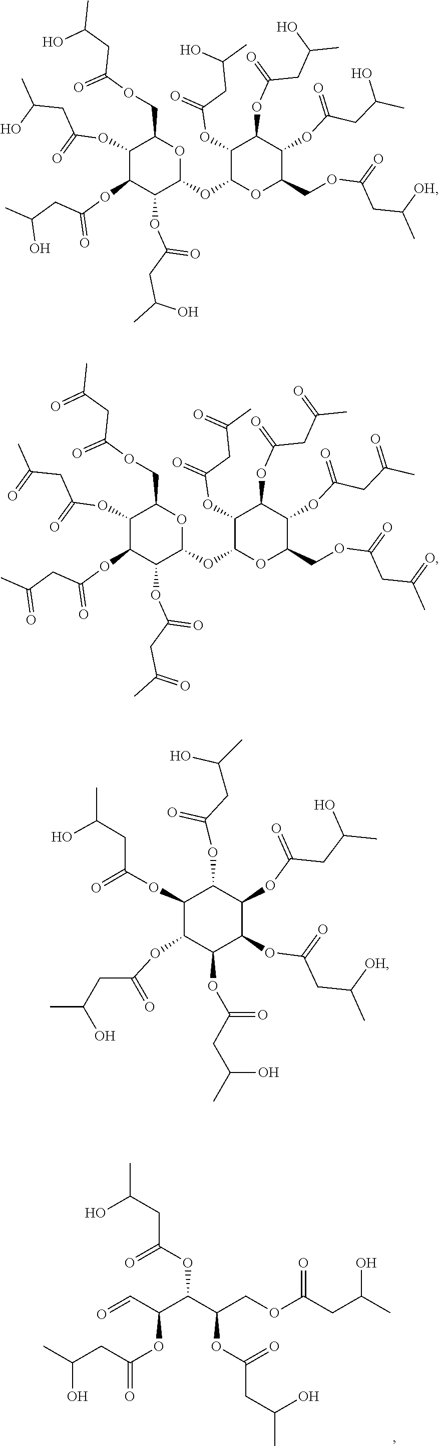

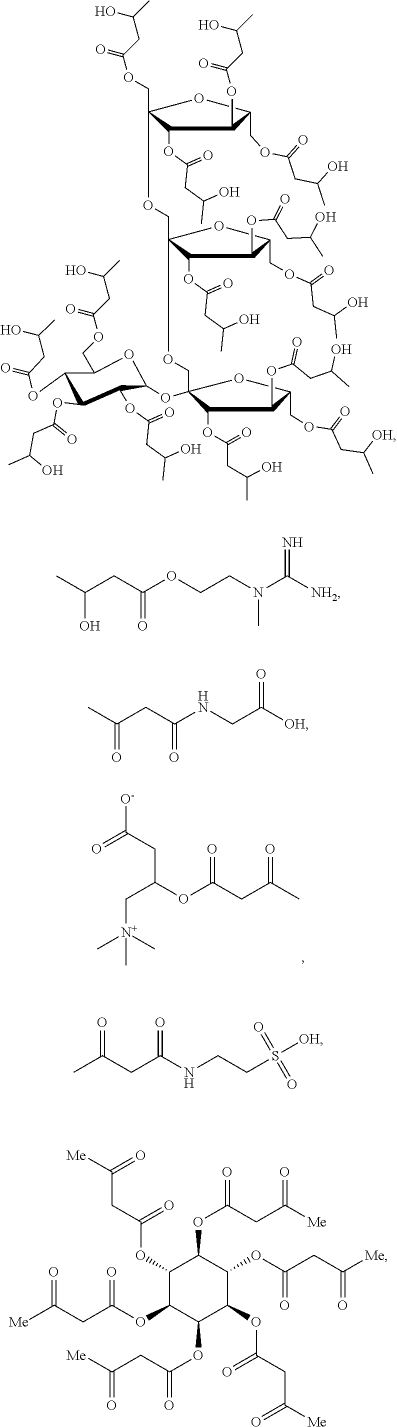

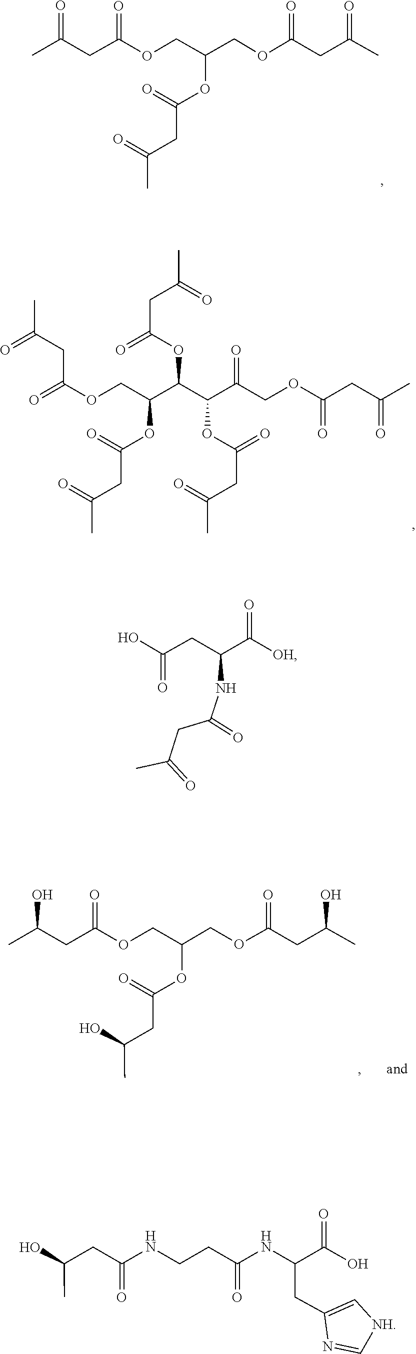

12. A pharmaceutical composition comprising a compound according to claim 1, and a pharmaceutically acceptable carrier, excipient, or diluent.

13. The pharmaceutical composition of claim 12, comprising at least two unique compounds according to claim 1.

14. The pharmaceutical composition of claim 13, wherein at least one compound comprises R² as β-hydroxybutyryl and at least one compound comprises R² as acetoacetyl.

15. The pharmaceutical composition of claim 14, wherein the at least two unique compounds are attached to the same R¹ carrier molecule.

16. (canceled)

17. A method of treating a glucose hypometabolic disorder in a subject, comprising administering to the subject a therapeutically effective amount of a compound according to claim 1.

18. The method of claim 17, wherein the glucose hypometabolic disorder is selected from the group consisting of Alzheimer's disease, Parkinson's disease, heart failure, type 2 diabetes, epilepsy, traumatic brain injury, amyotrophic lateral sclerosis, cancer cachexia, metabolic syndrome, and any condition in which the subject's tissue exhibits a quantifiable reduction in glucose utilization.

19. The method of claim 17, wherein the therapeutically effective amount is determined based on a measured glucose metabolism deficit (G_Diff) in one or more tissues of the subject.

20. (canceled)

21. The method of claim 17, further comprising administering at least one standard-of-care therapy for the glucose hypometabolic disorder.

22. The method of claim 17, wherein the administered compound provides an amount sufficient to raise the subject's blood beta-hydroxybutyrate (BHB) concentration to at least about 0.5 millimolar (mM).

23-25. (canceled)
